(19) United States
(12) Patent Application Publication
(10) Pub. No.: US 2025/0224408 A1
(43) Pub. Date: Jul. 10, 2025

(54) BIOMARKERS FOR DIAGNOSING ALZHEIMER'S DISEASE AND RELATED DEMENTIAS

(71) Applicants: Seer, Inc., Redwood City, CA (US); The General Hospital Corporation, Boston, MA (US)

(72) Inventors: Asim SIDDIQUI, San Francisco, CA (US); Steven E. ARNOLD, Swarthmore, PA (US)

(21) Appl. No.: 18/938,011

(22) Filed: Nov. 5, 2024

Related U.S. Application Data
(60) Provisional application No. 63/618,221, filed on Jan. 5, 2024.
(60) Provisional application No. 63/618,855, filed on Jan. 8, 2024.

(51) Int. Cl. G01N 33/68; G16B 25/10

(52) U.S. Cl. CPC G01N 33/6896; G01N 33/6848; G16B 25/10

(57) ABSTRACT

Provided herein are methods, compositions, and systems for diagnosing, assessing the likelihood of Alzheimer's disease, and assessing the rate of progression of Alzheimer's disease comprising assaying biofluid samples and identifying from the biofluid samples the presence/abundance of one or more biomarkers. Also provided herein are methods of assessing the likelihood of dementia progression or the rate of dementia progression comprising assaying biofluid samples and identifying from the biofluid samples the presence/abundance of one or more biomarkers.

### CROSS-REFERENCE

The present application claims the benefit of U.S. Provisional Application No. 63/618,221, filed Jan. 5, 2024; and U.S. Provisional Application No. 63/618,855, filed Jan. 8, 2024, both of which are incorporated herein by reference in their entirety.

### STATEMENT AS TO FEDERALLY SPONSORED RESEARCH

This invention was made with government support under R44 AG065051 awarded by the National Institutes of Health. The government has certain rights in the invention.

### BACKGROUND

Few methods exist for accurate neurodegenerative diagnosis. Primary screening for neurodegeneration is typically based on cognitive assessment (e.g., Mini-Mental State Examinations and Memory Impairment Screens), and therefore typically identifies cognitive decline without providing insight into underlying causes, pathologies, and risk factors. While medical imaging (e.g., Magnetic Resonance Imaging) and tissue analysis can, in certain cases, distinguish neurological conditions, these methods may struggle with early phase detection and tracking disease progression, and may be prohibitively invasive and cost intensive for routine use.

### SUMMARY

Responsive to the need for faster and less intensive methods for neurological disease diagnosis, aspects of the present disclosure provide compositions, systems, and methods for identifying pluralities of neurological disease biomarkers from biological samples. As individual biomarker analysis has proven to typically be ineffective for identifying neurological disease states, aspects of the present disclosure include methods which can identify tens, hundreds, thousands, or tens of thousands of biomolecules from biological samples, as well as patterns of biomolecule abundances and biomolecule-particle binding. Further disclosed herein are computer-implemented systems for identifying biological state information, for example neurological disease information, from biological data.

In some embodiments, provided herein are methods of assessing a likelihood of Alzheimer's disease (AD), the method comprising assaying a biofluid sample to obtain a data set comprising protein or peptide information; identifying, from the data set, an abundance of one or more biomarkers provided in TABLE 6 or TABLE 7; and assessing the likelihood of AD, based on the abundance of the one or more biomarkers in the biofluid sample.

In some embodiments, the one or more biomarkers comprise one or more of MBP, PLIN4, PKHG1, EIF1:EIF1B, CRBG3, ANTR1, PUR6, LUM, C1QT1, ENOB, DKK3, GPSM3, SYAC, HSP74, MICA1, APOD, LRC32, CSPG2, and OSTCN. In some embodiments, the one or more biomarkers comprise one or more of MBP, PLIN4, PKHG1, EIF1:EIF1B, CRBG3, ANTR1, PUR6, LUM, C1QT1, ENOB, DKK3, GPSM3, SYAC, HSP74, MICA1, LRC32, CSPG2, and OSTCN.

In some embodiments, provided herein are methods of diagnosing Alzheimer's disease, the method comprising contacting a biofluid sample from a subject with one or more physicochemically distinct particles to form a plurality of biomolecule coronas; obtaining a data set comprising protein or peptide information from the plurality of biomolecule coronas; identifying, from the data set, an abundance of one or more biomarkers provided in TABLE 6 or TABLE 7; and using a classifier to identify the biofluid sample as being indicative of a biological state comprising a healthy state or Alzheimer's disease (AD), based on the abundance of the one or more biomarkers in the data set.

In some embodiments, the one or more biomarkers comprise one or more of MBP, PLIN4, PKHG1, EIF1:EIF1B, CRBG3, ANTR1, PUR6, LUM, C1QT1, ENOB, DKK3, GPSM3, SYAC, HSP74, MICA1, APOD, LRC32, CSPG2, and OSTCN. In some embodiments, the one or more biomarkers comprise MBP, PLIN4, PKHG1, EIF1:EIF1B, CRBG3, ANTR1, PUR6, LUM, C1QT1, ENOB, DKK3, GPSM3, SYAC, HSP74, MICA1, LRC32, CSPG2, and OSTCN. In some embodiments, the one or more biomarkers comprise one or more of MBP and OSTCN.

In some embodiments, the one or more biomarkers comprise two or more biomarkers. In some embodiments, the one or more biomarkers comprise three or more biomarkers. In some embodiments, the one or more biomarkers comprise four or more biomarkers.

In some embodiments, the one or more biomarkers further comprise an additional biomarker comprising pTau (e.g., pTau-181 or pTau-217).

In some embodiments, the classifier comprises a machine learning algorithm. In some embodiments, the machine learning algorithm comprises a logistic regression-based machine learning model.

Provided herein in some embodiments, is a method for assessing a likelihood of dementia progression, the method comprising assaying a biofluid sample to obtain a data set comprising protein or peptide information; identifying, from the data set, an abundance of one or more biomarkers; and assessing a rate of dementia progression in the biofluid sample, based on the abundance of the one or more biomarkers.

Also provided herein in some embodiments, is a method for assessing a likelihood of dementia progression, the method comprising contacting a biofluid sample from a subject with one or more physicochemically distinct particles to form a plurality of biomolecule coronas; obtaining a data set comprising protein or peptide information from the plurality of biomolecule coronas; identifying, from the data set, an abundance of one or more biomarkers; and using a classifier to track a rate of dementia progression in the biofluid sample, based on the abundance of the one or more biomarkers in the data set.

In some embodiments, the one or more biomarkers comprise one or more of CRISPLD2, CLNS1A, BLVRB, SMYD5, PRPS1, SELENBP1, OXSR1, VGF, and GOLPH3. In some embodiments, CRISPLD2, CLNS1A, BLVRB, SMYD5, PRPS1, SELENBP1, OXSR1, or a combination thereof are associated with the biofluid sample having an increased rate of dementia progression. In some embodiments, the CRISPLD2, CLNS1A, or a combination thereof are associated with the biofluid sample having an increased rate of dementia progression. In some embodiments, an increased rate of dementia progression is associated with a shorter time to clinical dementia rating global (CDRg) increase. In some embodiments, GOLPH3, VGF, or a combination thereof are associated with the biofluid sample having a decreased rate of dementia progression. In some embodiments, GOLPH3 is associated with the biofluid sample having a decreased rate of dementia progression. In some embodiments, a decreased rate of dementia progression is associated with a delay in CDRg increase.

In some embodiments, the classifier comprises time-to-event analysis. In some embodiments, the classifier comprises Cox proportion hazards (CPH) models, Cox time-varying (CTV) regression models, or a combination thereof. In some embodiments, the classifier comprises CTV regression models.

In some embodiments, the physicochemically distinct particles comprise lipid particles, metal particles, silica particles, or polymer particles. In some embodiments, the physicochemically distinct particles comprise polystyrene particles, magnetizable particles, dextran particles, silica particles, dimethylamine particles, carboxylate particles, amino particles, benzoic acid particles, or agglutinin particles.

In some embodiments, contacting comprises incubating the biofluid sample with the one or more physicochemically distinct particles. In some embodiments, contacting comprises incubating the biofluid sample with the one or more physicochemically distinct particles for about 1 hour.

In some embodiments, obtaining the data set comprises detecting proteins of the biomolecule coronas by mass spectrometry, chromatography, liquid chromatography, high-performance liquid chromatography, solid-phase chromatography, a lateral flow assay, an immunoassay, an enzyme-linked immunosorbent assay, a western blot, a dot blot, or immunostaining, or a combination thereof. In some embodiments, obtaining the data set comprises detecting the proteins of the biomolecule coronas by mass spectrometry. In some embodiments, obtaining the data set comprises detecting the proteins of the biomolecule coronas by liquid chromatography mass spectrometry (LC-MS). In some embodiments, obtaining the data set comprises measuring a readout indicative of the presence, absence, or amount of proteins of the biomolecule coronas.

In some embodiments, the biofluid sample comprises a blood sample, a serum sample, or a plasma sample. In some embodiments, the biofluid sample comprises a blood sample that has had red blood cells removed. In some embodiments, the biofluid is plasma.

In some embodiments, the one or more physicochemically distinct particles comprise 2 or more physicochemically distinct particles.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a computer system that is programmed or otherwise configured to implement methods provided herein, in accordance with some embodiments.

FIG. 2 provides a workflow for collecting biomolecules from a biological sample onto particles, in accordance with some embodiments.

FIG. 3 provides a workflow for a particle-based assay for analyzing biomolecules from a biological sample, in accordance with some embodiments.

FIG. 4 illustrates a workflow utilizing assay instrumentation and materials and a computer-implemented system for biological state analysis.

FIG. 5 shows an example of study design, plasma proteomics quantifications, and cohort subsetting for biomarker discovery of AD and forecasting rate of cognitive decline.

FIG. 6A shows 1D annotation enrichment analysis for AD. 1D score was calculated for KEGG and GOBP terms as described in Example 1. The p-values are adjusted based on Benjamini-Hochberg (BH) multiple testing correction and filtered at 5% FDR. FIG. 6B shows differential expression analysis for AD vs. Normal (No neurodegenerative disease). Volcano plot showing log 2 fold change of proteins (x axis) and −log 10 p-values after BH multiple testing correction (y axis). The upregulated proteins are depicted on the right; the downregulated proteins are depicted on the left. FIG. 6C shows Dynamic range of identified proteins matched with HPPP database.

FIG. 7A shows a flowchart of our machine learning pipeline for our “pTau+Prot” model. Some nodes have hyperparameters listed, which are tuned using nested cross validation. FIG. 7B shows results of 10 fold cross validation comparing the AUROC and Average Precision of the Rule-of-thumb baseline classifier, pTau-181 concentration alone, and the model shown in FIG. 7A. FIG. 7C shows the top 20 average coefficient values for the pTau+Prot model (when sorted by absolute values) across the 10 folds. Error bars indicate 95% confidence internal estimated using 1000 bootstraps with replacement.

FIGS. 8A and 8B show two examples of subjects' CDR history. All periods represented in a Cox time-varying model between obtained blood samples and before an event are characterized as E=0 and the period between last blood draw and an observed event (CDRg increase) is characterized as E=1. A subject must have at least three visits without a CDRg increase for the final period to be labeled as censored (E=0). FIG. 8C shows a CTV volcano plot where the y-axis is −log 10 adjusted p-value (BH-corrected) and the dashed line represents adjusted p-value of 0.05. Proteins with positive coefficients are indicative of proteins associated with increased risk of time-to-CDRg increase while those with negative coefficients are associated with decreased risk. FIG. 8D shows CRISPLD2 survival curve generated from a CPH model between last draw and event as an example of a positive coefficient. FIG. 8E shows GOLPH3 survival curve generated from a CPH model between last draw and event, as an example of a negative coefficient.

### DETAILED DESCRIPTION

From a molecular perspective, neurological disease progression is often difficult to assess, as neurodegeneration is typically associated with multiple underlying and often independent causes. For example, presently recognized mild cognitive impairment (MCI) and Alzheimer's disease (AD) risk factors and indicators may include vascular damage, hypertension, atherosclerosis, infection (including numerous forms of herpes simplex infections), personality changes, cognitive decline, or metabolic abnormalities, with some researchers even positing Alzheimer's disease as “Type 3” diabetes. As many neurological disease risk factors and indicators overlap with those of non-neurological conditions (e.g., liver disease and cirrhosis), identifying and distinguishing neurological diseases is often infeasible with standard pathological and biomarker analysis methods. Further complicating neurological disease analysis, neurological diseases may manifest negligible changes outside of affected tissues, rendering many forms of non-intensive (e.g., blood-based) neurological disease analysis poorly prognostic. Additionally, while blood-based biomarkers of amyloid beta and phosphorylated tau are emerging with improving accuracy to predict brain AD pathology, their utility for disease staging or prognosis is still limited. Accordingly, options for neurological disease diagnoses (e.g., for ADRD) absent expensive imaging and intensive nerve biopsy analyses have remained limited.

Dementia affects over 55 million people worldwide, with Alzheimer's Disease (AD) and Related Dementias (ADRD) being the most common forms. However, heterogeneity in presentation and rates of cognitive decline and disease progression, as well as the need for more informative and accessible biomarkers, contribute to challenges in diagnosis and prognosis.

Responsive to the need for rapid, accurate, and minimally intensive neurological disease diagnostics, the present disclosure provides a range of compositions, systems, and methods for assessing neurological diseases (e.g., ADRD) from patient samples. In some cases, the compositions, systems, and methods may be configured to utilize blood or components thereof (e.g., whole blood, plasma, serum) to determine the presence of a neurological disease, such as Alzheimer's disease or dementia. The methods, systems, and compositions of the present disclosure may identify a plurality of biomolecules from samples and may furthermore determine relative or absolute abundances of at least a subset of the biomolecules. This may be compared to other blood biomarker tests, some of which may be used identify only a single biomolecule (e.g., a particular protein) from blood samples.

In some embodiments, a method of the present disclosure includes methods of identifying a biological state of a biofluid sample. In some embodiments, a method of the present disclosure includes methods of identifying risk of developing a biological state of a biofluid sample. In some embodiments, the methods of the present disclosure include methods of identifying risk of developing a biological state of a biofluid sample.

In some embodiments, the biological state comprises a healthy state, dementia, pre-Alzheimer's disease, or Alzheimer's disease. In some embodiments, the biological state comprises a healthy state or Alzheimer's disease. In some embodiments, the biological state comprises a healthy state. In some embodiments, the biological state comprises dementia. In some embodiments, the biological state comprises pre-Alzheimer's disease. In some embodiments, the biological state comprise Alzheimer's disease.

A method of the present disclosure may comprise assessing a likelihood of Alzheimer's disease (AD). In some embodiments, a method of the present disclosure comprises diagnosing Alzheimer's disease (e.g., identifying a biofluid sample as being indicative of a biological state comprising AD). In other embodiments, a method of the present disclosure comprises identifying increased likelihood of a biofluid sample being indicative of AD. In some embodiments, a method of the present disclosure comprises assessing risk of development of AD.

A method of the present disclosure may comprise assessing likelihood of dementia progression. In some embodiments, the methods of the present disclosure may comprise assessing risk of dementia progression. In some embodiments, the methods of the present disclosure may comprise assessing the rate of dementia progression.

In some embodiments, the rate of dementia progression is classified as fast dementia progression (e.g., biomarkers are associated with a short time to CDRg increase). In some embodiments, the rate of dementia progression is classified as slow dementia progression. Slow dementia progression may include instances where none of the biomarkers described herein are identified. In some instances, slow dementia progression may include instances where biomarkers associated with a delayed time to CDRg increase are identified.

In some embodiments, the methods comprise contacting a biofluid sample (e.g., plasma) form a subject with one or more physicochemically distinct particles to form a plurality of biomolecule corona. In some embodiments, the plurality of biomolecule corona are assayed to obtain a data set comprising protein or peptide information from the plurality of biomolecule corona.

In some embodiments, the methods provided herein comprise assaying a biofluid sample. In some instances, assaying comprises assaying as described elsewhere herein. In some embodiments, assaying comprises mass spectrometry, liquid chromatography mass spectrometry (LC-MS), or LC-MS/MS.

In some embodiments, assaying the biofluid sample provides a data set comprising protein or peptide information. In some instances, the protein or peptide information comprises a biomarker as described elsewhere herein, such as in TABLE 6, TABLE 7, or TABLE 8. In some instances, the protein or peptide information does not comprise a biomarker as described herein.

In some embodiments, the methods provided herein comprise identifying an abundance and/or presence of a subset of biomarkers (e.g., from the data set) selected from TABLE 6, TABLE 7, or TABLE 8, or a combination therefrom. In some embodiments, the methods herein comprise identifying an abundance and/or presence of a subset of biomarkers selected from TABLE 6. In some embodiments, the methods comprise identifying an abundance and/or presence of a subset of biomarkers selected from TABLE 7. In some embodiments, the methods provided herein comprise identifying an abundance and/or presence of a subset of biomarkers selected from TABLE 8. In some embodiments, the biomarkers comprise MBP, PLIN4, PKHG1, EIF1:EIF1B, CRBG3, ANTR1, PUR6, LUM, C1QT1, ENOB, DKK3, GPSM3, SYAC, HSP74, MICA1, APOD, LRC32, CSPG2, and OSTCN. In some embodiments, the biomarkers comprise MBP, PLIN4, PKHG1, EIF1:EIF1B, CRBG3, ANTR1, PUR6, LUM, C1QT1, ENOB, DKK3, GPSM3, SYAC, HSP74, MICA1, LRC32, CSPG2, and OSTCN. In some embodiments, the biomarkers comprise MBP and OSTCN. In some embodiments, the biomarkers comprise CRISPLD2, CLNS1A, BLVRB, SMYD5, PRPS1, SELENBP1, OXSR1, VGF, and GOLPH3. In some embodiments, the biomarkers comprise CRISPLD2 or CLNS1A. In some embodiments, the biomarkers comprise GOLPH3 or VGF. In some embodiments, the biomarkers comprise GOLPH3. In some embodiments, the one or more biomarkers further comprise an additional biomarker comprising pTau (e.g., pTau-181 or pTau-217). In some embodiments, the pTau is pTau-181. In some embodiments, the pTau is pTau-217.

In some embodiments, the methods herein comprise identifying the presence/abundance of one or more biomarkers. In some embodiments, the methods herein comprise identifying the presence/abundance of two or more biomarkers. In some embodiments, the methods herein comprise identifying the presence/abundance of three or more biomarkers. In some embodiments, the methods herein comprise identifying the presence/abundance of four or more biomarkers. In some embodiments, the methods herein comprise identifying the presence/abundance of five or more biomarkers. In some embodiments, the methods herein comprise identifying the presence/abundance of six or more biomarkers. In some embodiments, the methods herein comprise identifying the presence/abundance of seven or more biomarkers.

In some embodiments, the methods provided herein comprise assessing the likelihood of AD, based on the abundance and/or presence of the one or more biomarkers in the biofluid sample. In some embodiments, the methods herein comprise diagnosing AD based on the abundance and/or presence of one or more biomarkers in a biofluid sample. In some embodiments, the methods herein comprise identifying increased likelihood of a biofluid sample being indicative of AD, based on the presence and/or abundance of one or more biomarkers in a biofluid sample. In some embodiments, the methods comprise assessing risk of developing AD, based on the presence and/or abundance of one or more biomarkers in a biofluid sample.

In some embodiments, the methods provided herein comprise using a classifier to identify the likelihood of AD, based on the abundance and/or presence of the one or more biomarkers in the biofluid sample. In some embodiments, the methods herein comprise using a classifier to diagnose AD based on the abundance and/or presence of one or more biomarkers in a biofluid sample. In some embodiments, the methods herein comprise using a classifier to identify increased likelihood of a biofluid sample being indicative of AD, based on the presence and/or abundance of one or more biomarkers in a biofluid sample. In some embodiments, the methods comprise using a classifier to identify risk of developing AD, based on the presence and/or abundance of one or more biomarkers in a biofluid sample.

In some embodiments, the biomarkers comprising CRISPLD2, CLNS1A, or a combination thereof are associated with the biofluid sample having an increased rate of dementia progression. In some embodiments, the biomarkers comprising CRISPLD2, CLNS1A, or a combination thereof are associated with the biofluid sample having an increased likelihood of dementia progression. In some embodiments, the biomarkers comprising CRISPLD2, CLNS1A, or a combination thereof are associated with the biofluid sample having an increased risk of dementia progression.

In some embodiments, the biomarkers GOLPH3, VGF, or a combination thereof are associated with the biofluid sample having a decreased rate of dementia progression. In some embodiments, the biomarkers GOLPH3, VGF, or a combination thereof are associated with the biofluid sample having a decreased likelihood of dementia progression. In some embodiments, the biomarkers GOLPH3, VGF, or a combination thereof are associated with the biofluid sample having a decreased risk of dementia progression.

In some embodiments, the rate, risk, and/or likelihood of dementia progression is associated with clinical dementia rating global (CDRg) increases or decreases, such that the presence of certain biomarkers may be associated with shorter times to CDRg increases (e.g., increased rate of dementia progression) and others are associated with delayed times to CDRg increase (e.g., decreased rate of dementia progression). The Clinical Dementia Rating (CDR) scale is a widely used dementia staging instrument, yielding a global score and a summated score.

In some embodiments, the methods comprise assessing a rate of likelihood of dementia progression based on the presence and/or abundance of one or more biomarkers. In some embodiments, the methods comprise assessing likelihood of dementia progression based on the presence and/or abundance of one or more biomarkers. In some embodiments, the methods comprise assessing the risk of dementia progression based on the presence and/or abundance of one or more biomarkers. In some embodiments, the methods comprise tracking a rate of dementia progression based on the presence and/or abundance of one or more biomarkers.

In some embodiments, the methods comprise using a classifier to track a rate of likelihood of dementia progression based on the presence and/or abundance of one or more biomarkers. In some embodiments, the methods comprise using a classifier to assess the likelihood of dementia progression based on the presence and/or abundance of one or more biomarkers. In some embodiments, the methods comprise using a classifier to assess the risk of dementia progression based on the presence and/or abundance of one or more biomarkers. In some embodiments, the methods comprise using a classifier to track a rate of dementia progression based on the presence and/or abundance of one or more biomarkers.

A method of the present disclosure may comprise contacting a biological sample (e.g., plasma) with a particle under conditions suitable for biomolecule collection (e.g., non-covalent adsorption of a protein) on the particle. The collection of biomolecules on the surface of the particle may be referred to as a ‘biomolecule corona’. The biomolecule corona that forms on a particle may comprise a complex mixture of biomolecules from the biological sample. A biomolecule corona may include nucleic acids, small molecules, proteins, lipids, polysaccharides, or any combination thereof. The biomolecule corona may compress the abundance ratios of biomolecules from a sample, thereby enabling analysis of dilute, and in many cases difficult to analyze, biomolecules.

A method of the present disclosure may comprise fractionating a biological sample with a particle. In some cases, the method comprises contacting the biological sample with the particle to form thereon a biomolecule corona which comprises biomolecules from the biological sample. The method may comprise separating the biomolecule corona from the biological sample, for example by immobilizing (e.g., magnetically trapping) the particle within a volume and removing unbound components of the biological sample from the volume (e.g., through a series of wash steps). The method may also comprise analyzing a biomolecule of the biomolecule corona. The analyzing may identify the biomolecule, determine an abundance of the biomolecule, identify a state (e.g., post-transcriptional processing of RNA or a post-translational modification of a protein) or form (e.g., a conformation) of the biomolecule, or identify a biomolecule-biomolecule interaction (e.g., a protein-protein interaction reflected, for example, by the formation of a multi-protein complex). As a biomolecule corona may comprise a compressed dynamic range relative to a sample, the analyzing may identify biomolecules over a broader dynamic range (in terms of biological sample concentrations of the biomolecules) than if the analyzing were performed directly on the biological sample (e.g., without particle-based fractionation of the biological sample).

In some cases, the method comprises contacting the biological sample with a plurality of (e.g., physicochemically distinct) particles. As biomolecule corona composition may depend on a number of factors, including biological sample composition, biological sample conditions (e.g., pH and salinity), particle concentration, and particle physicochemical properties (e.g., surface charge, hydrophilicity, density, roughness), contacting a sample with a plurality of (e.g., physicochemically distinct) particles may generate a plurality of biomolecule coronas which reflect different characteristics of the sample. For example, a biomolecule corona of a first particle may be sensitive to sample lipid levels, while a biomolecule of a second particle may be sensitive to nanomolar-scale changes in cytokine concentrations. Furthermore, two biomolecule coronas may comprise different subsets of biomolecules from a sample. Accordingly, the method may not only identify a plurality of biomolecules from a biological sample, but may also generate additional information by identifying one or more relationships between biomolecule corona composition, particle type, and sample conditions.

Aspects of the present disclosure provide compositions, systems, and methods for collecting biomolecules on particles, as well as particle panels of multiple distinct particle types, which may enrich proteins from a sample onto distinct biomolecule coronas formed on the surface of the distinct particle types. The particle panels disclosed herein can be used in methods of corona analysis to detect tens, hundreds, thousands, or tens of thousands of proteins across a wide dynamic range in the span of hours. In some cases, the composition, system, or method may utilize one particle. In some cases, the composition, system, or method may utilize at least two particles. In some cases, the composition, system, or method may utilize at least three particles. In some cases, the composition, system, or method may utilize at least four particles. In some cases, the composition, system, or method may utilize at least five particles. In some cases, the composition, system, or method may utilize at least six particles. In some cases, the composition, system, or method may utilize at least eight particles. In some cases, the composition, system, or method may utilize at least ten particles. In some cases, the composition, system, or method may utilize at least twelve particles. In some cases, the composition, system, or method may utilize at least fifteen particles. In some cases, the composition, system, or method may contact a sample with a particle under at least two conditions (e.g., at least two temperatures), and may compare the biomolecule corona formed under each of the at least two conditions. In some cases, the method may comprise identifying an abundance ratio of a biomolecule on two or more particles. In some cases, the method may comprise identifying an abundance ratio of a plurality of biomolecules on a particle. In some cases, the method may comprise identifying an abundance ratio of a first biomolecule on a first particle and a second biomolecule on a second particle.

In some aspects of the present disclosure, the methods and systems are not limited to particle-based biomarker detection methods. In some embodiments, the methods and systems provided herein comprise assaying a biofluid sample, such as using any suitable technique such as described elsewhere herein. In some embodiments, a data set is obtained by the assaying of the biofluid sample, wherein the data set comprises protein or peptide information (e.g., including information related to that of the biomarkers described herein). In some embodiments, the methods comprise identifying from the data set an abundance of one or more biomarkers. The biomarkers may be associated with any of the neurodegenerative diseases described herein, such as AD or dementia. In some instances, the abundance of the biomarkers may be associated with increased risk of AD (e.g., a higher abundance of a biomarker described herein may be associated with increased risk of the biofluid being indicative of AD). In some embodiments, the abundance of biomarkers may be associated with increased risk/likelihood of dementia progression (e.g., a higher abundance of a biomarker described herein may be associated with increased risk/likelihood of dementia progression). In some embodiments, the abundance of biomarkers may be associated with decreased risk/likelihood of dementia progression (e.g., a higher abundance of a biomarker or a lower abundance of a biomarker described herein may be associated with decreased risk/likelihood of dementia progression). In some instances, the abundance of biomarkers described herein may also be used to assess a rate of dementia progression. In some embodiments, an increased abundance of biomarkers may be associated with an increased rate of dementia progression. In some embodiments, a decreased abundance of biomarkers may be associated with a decreased rate of dementia progression. In some embodiments, an increased abundance of biomarkers (e.g., GOLPH3 or VGF) may be associated with decreased rate of dementia progression.

In some embodiments, such as described elsewhere herein, the assaying may include any suitable technique capable of characterizing (e.g., identify, measure, and/or quantify) the proteins and peptides present in the biofluid sample. In some embodiments, assaying comprises mass spectrometry (MS), liquid chromatography-mass spectrometry (LC-MS), protein sequencing, light scattering (e.g., dynamic light scattering (DLS), static light scattering (SLS), or circular dichroism (CD), affinity-based detection methods (e.g., enzyme-linked immunosorbent assay (ELISA), proximity extension assays, or aptamer-based detection) or any combination thereof. In some embodiments, assaying comprises mass spectrometry, liquid chromatography-mass spectrometry (LC-MS), protein sequencing, or any combination thereof. In some embodiments, assaying comprises mass spectrometry. In some embodiments, assaying comprises liquid chromatography-mass spectrometry (LC-MS). In some embodiments, assaying comprises protein sequencing. In some embodiments, assaying may comprise high throughput single molecule protein sequencing. For example, peptides may be sequenced using TIME DOMAIN SEQUENCING from Quantum-SI. In some embodiments, assaying comprises targeted mass spectrometry, such as multiple reaction monitoring (MRM). In some embodiments, assaying comprises top-down mass spectrometry. In some embodiments, assaying comprises bottom-up mass spectrometry.

### Particle Properties and Types

Particle types consistent with the methods disclosed herein can be made from various materials. For example, particle materials of the present disclosure may include metals, polymers, magnetic materials, and lipids. Magnetic particles may be iron oxide particles. Examples of metals include any one of gold, silver, copper, nickel, cobalt, palladium, platinum, iridium, osmium, rhodium, ruthenium, rhenium, vanadium, chromium, manganese, niobium, molybdenum, tungsten, tantalum, iron, cadmium, any other material described in U.S. Pat. No. 7,749,299, or any combination thereof. In some cases, a particle may be a superparamagnetic iron oxide nanoparticle (SPION). A magnetic particle may be a ferromagnetic particle, a ferrimagnetic particle, a paramagnetic particle, a superparamagnetic particle, or any combination thereof (e.g., a particle may comprise a ferromagnetic material and a ferrimagnetic material). For example, a particle core may comprise superparamagnetic γ-ferric iron oxide. In some cases, a particle may comprise a distinct core (e.g., the innermost portion of the particle), shell (e.g., the outermost layer of the particle), and shell or shells (e.g., portions of the particle disposed between the core and the shell). In some cases, a core may comprise a metal, an oxide, a nitride, a ceramic, a carbon material, a silicon material, a polymer, or any combination thereof. In some cases, a shell may comprise a polymer, a saccharide, a lipid, a peptide, a self-assembled monolayer, a sol-gel, a hydrogel, a glass, or any combination thereof. In some cases, a shell may comprise polystyrene, N-(3-(Dimethylamino) propyl) methacrylamide (DMAPMA), or a combination thereof. In some cases, a shell material may comprise a small molecule functionalization. In some cases, a shell material may comprise a biomolecular functionalization (e.g., a peptide or saccharide functional appendage). In some cases, a particle may comprise a uniform composition. In some cases, a core or a shell may comprise a plurality of materials comprising a degree of phase separation. For example, a shell may comprise two phase separated polymers. In some cases, a particle core and shell may comprise different densities. In some cases, a shell material may comprise a thickness of at least 2 nm, at least 4 nm, at least 5 nm, at least 8 nm, at least 10 nm, at least 15 nm, at least 20 nm, at least 25 nm, at least 30 nm, or at least 35 nm. In some cases, a shell material may comprise a thickness of at most 35 nm, at most 30 nm, at most 25 nm, at most 20 nm, at most 15 nm, at most 10 nm, at most 8 nm, at most 5 nm, at most 4 nm, or at most 2 nm.

In some cases, a particle may comprise a polymer. In some cases, the polymer may constitute a core material (e.g., the core of a particle may comprise a particle), a layer (e.g., a particle may comprise a layer of a polymer disposed between its core and its shell), a shell material (e.g., the surface of the particle may be coated with a polymer), or any combination thereof. In some cases, the polymer may comprise a polyethylene, a polycarbonate, a polyanhydride, a polyhydroxyacid, a polypropylfumerate, a polycaprolactone, a polyamide, a polyacetal, a polyether, a polyester, a poly(orthoester), a polycyanoacrylate, a polyvinyl alcohol, a polyurethane, a polyphosphazene, a polyacrylate, a polymethacrylate, a polycyanoacrylate, a polyurea, a polystyrene, a polyamine, a polyalkylene glycol (e.g., polyethylene glycol (PEG)), a polyester (e.g., poly(lactide-co-glycolide) (PLGA) or a polylactic acid), a copolymer of two or more polymers (e.g., a copolymer of a polyalkylene glycol (e.g., PEG) and a polyester (e.g., PLGA)), or any combination thereof. In some cases, the polymer may be a lipid-terminated polyalkylene glycol and a polyester, or any other material disclosed in U.S. Pat. No. 9,549,901.

In some cases, a particle may comprise a lipid. In some cases, a lipid-containing particle may comprise a lipid coupled to its surface (e.g., covalently attached to a surface amine of the particle or non-covalently bound by a particle-bound lipid binding protein). In some cases, a lipid-containing particle may comprise a lipid within a monolayer or bilayer comprising the lipid. In some cases, the lipid monolayer or bilayer may comprise non-lipidic biomolecules, including sterols, proteins (e.g., clathrins), and saccharides. In some cases, a plurality of lipids associated with a particle may be fully or partially polymerized. In some cases, a particle may comprise a liposome. Examples of lipids that can be used to form the particles of the present disclosure include cationic, anionic, and neutrally charged lipids. In some cases, particles can be made of any one of dioleoylphosphatidylglycerol (DOPG), diacylphosphatidylcholine, diacylphosphatidylethanolamine, ceramide, sphingomyelin, cephalin, cholesterol, cerebrosides and diacylglycerols, dioleoylphosphatidylcholine (DOPC), dimyristoylphosphatidylcholine (DMPC), dioleoylphosphatidylserine (DOPS), phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, cholesterol, any other material listed in U.S. Pat. No. 9,445,994 (which is incorporated herein by reference in its entirety), or any combination thereof.

Examples of particles of the present disclosure are provided in TABLE 1.

TABLE 1
Example particles of the present disclosure

| Batch No. | Type | Particle ID | Description |
|---|---|---|---|
| S-001-001 | HX-13 | SP-001 | Carboxylate (Citrate) superparamagnetic iron oxide NPs |
| | | | (SPION) |
| S-002-001 | HX-19 | SP-002 | Phenol-formaldehyde coated SPION |
| S-003-001 | HX-20 | SP-003 | Silica-coated superparamagnetic iron oxide NPs |
| | | | (SPION) |
| S-004-001 | HX-31 | SP-004 | Polystyrene coated SPION |
| S-005-001 | HX-38 | SP-005 | Carboxylated Poly(styrene-co-methacrylic acid), P(St- |
| | | | co-MAA) coated SPION |
| S-006-001 | HX-42 | SP-006 | N-(3-Trimethoxysilylpropyl)diethylenetriamine coated |
| | | | SPION |
| S-007-001 | HX-56 | SP-007 | poly(N-(3-(dimethylamino)propyl) methacrylamide) |
| | | | (PDMAPMA)-coated SPION |
| S-008-001 | HX-57 | SP-008 | 1,2,4,5-Benzenetetracarboxylic acid coated SPION |
| S-009-001 | HX-58 | SP-009 | poly(vinylbenzyltrimethylammonium chloride) |
| | | | (PVBTMAC) coated SPION |
| S-010-001 | HX-59 | SP-010 | Carboxylate, PAA coated SPION |
| S-011-001 | HX-86 | SP-011 | poly(oligo(ethylene glycol) methyl ether methacrylate) |
| | | | (POEGMA)-coated SPION |
| P-033-001 | P33 | SP-333 | Carboxylate microparticle, surfactant free |
| P-039-003 | P39 | SP-339 | Polystyrene carboxyl functionalized |
| P-041-001 | P41 | SP-341 | Carboxylic acid |
| P-047-001 | P47 | SP-365 | Silica |
| P-048-001 | P48 | SP-348 | Carboxylic acid, 150 nm |
| P-053-001 | P53 | SP-353 | Amino surface microparticle, 0.4-0.6 μm |
| P-056-001 | P56 | SP-356 | Silica amino functionalized microparticle, 0.1-0.39 μm |
| P-063-001 | P63 | SP-363 | Jeffamine surface, 0.1-0.39 μm |
| P-064-001 | P64 | SP-364 | Polystyrene microparticle, 2.0-2.9 μm |
| P-065-001 | P65 | SP-365 | Silica |
| P-069-001 | P69 | SP-369 | Carboxylated Original coating, 50 nm |
| P-073-001 | P73 | SP-373 | Dextran based coating, 0.13 μm |
| P-074-001 | P74 | SP-374 | Silica Silanol coated with lower acidity |
| — | S-118 | SP-118 | 1,6-hexanediamine functionalized SPION |
| — | S-125 | SP-125 | Amine functionalized silica-coated SPION |
| — | S-128 | SP-128 | Mixed amide, carboxylate functionalized, silica-coated |
| | | | SPION |
| — | S-199 | SP-199 | Epichlorohydrin crosslinked Dextran-coated SPION |
| — | S-229 | SP-229 | N¹-(3-(trimethoxysilyl)propyl)hexane-1,6-diamine |
| | | | functionalized, silica-coated SPION |

A particle of the present disclosure may be synthesized, or a particle of the present disclosure may be purchased from a commercial vendor. For example, some particles of the present disclosure may be purchased from commercial vendors including Sigma-Aldrich, Life Technologies, Fisher Biosciences, nanoComposix, Nanopartz, Spherotech, and other commercial vendors. In some cases, a particle of the present disclosure may be purchased from a commercial vendor and further modified, coated, or functionalized.

An example of a particle type of the present disclosure may be a carboxylate (Citrate) superparamagnetic iron oxide nanoparticle (SPION), a phenol-formaldehyde coated SPION, a silica-coated SPION, a polystyrene coated SPION, a carboxylated poly(styrene-co-methacrylic acid) coated SPION, a N-(3-Trimethoxysilylpropyl) diethylenetriamine coated SPION, a poly(N-(3-(dimethylamino) propyl) methacrylamide) (PDMAPMA)-coated SPION, a 1,2,4,5-Benzenetetracarboxylic acid coated SPION, a poly(Vinylbenzyltrimethylammonium chloride) (PVBTMAC) coated SPION, a carboxylate, PAA coated SPION, a poly(oligo (ethylene glycol) methyl ether methacrylate) (POEGMA)-coated SPION, a carboxylate microparticle, a polystyrene carboxyl functionalized particle, a carboxylic acid coated particle, a silica particle, a carboxylic acid particle of about 150 nm in diameter, an amino surface microparticle of about 0.4-0.6 μm in diameter, a silica amino functionalized microparticle of about 0.1-0.39 μm in diameter, a Jeffamine surface particle of about 0.1-0.39 μm in diameter, a polystyrene microparticle of about 2.0-2.9 μm in diameter, a silica particle, a carboxylated particle with an original coating of about 50 nm in diameter, a particle coated with a dextran based coating of about 0.13 μm in diameter, or a silica silanol coated particle with low acidity. An example of a particle type of the present disclosure may be a mixed amide, carboxylate functionalized, silica-coated SPION having a mean size of about 280 nm and a zeta potential of about 50 mV. An example of a particle type of the present disclosure may be an epichlorohydrin crosslinked Dextran-coated SPION having a mean size of about 275+/−30 nm and a zeta potential of about 15 to 20 mV. An example of a particle type of the present disclosure may be a N¹-(3-(trimethoxysilyl) propyl) hexane-1,6-diamine functionalized, silica-coated SPION having a mean size of about 280 nm and a zeta potential of about 40 mV.

Particles of the present disclosure can be made and used in methods of forming protein coronas after incubation in a biofluid at a wide range of sizes. In some cases, a particle of the present disclosure may be a nanoparticle. In some cases, a nanoparticle of the present disclosure may be from about 10 nm to about 1000 nm in diameter. In some cases, a nanoparticle may be at least 10 nm, at least 100 nm, at least 200 nm, at least 300 nm, at least 400 nm, at least 500 nm, at least 600 nm, at least 700 nm, at least 800 nm, at least 900 nm, from 10 nm to 50 nm, from 50 nm to 100 nm, from 100 nm to 150 nm, from 150 nm to 200 nm, from 200 nm to 250 nm, from 250 nm to 300 nm, from 300 nm to 350 nm, from 350 nm to 400 nm, from 400 nm to 450 nm, from 450 nm to 500 nm, from 500 nm to 550 nm, from 550 nm to 600 nm, from 600 nm to 650 nm, from 650 nm to 700 nm, from 700 nm to 750 nm, from 750 nm to 800 nm, from 800 nm to 850 nm, from 850 nm to 900 nm, from 100 nm to 300 nm, from 150 nm to 350 nm, from 200 nm to 400 nm, from 250 nm to 450 nm, from 300 nm to 500 nm, from 350 nm to 550 nm, from 400 nm to 600 nm, from 450 nm to 650 nm, from 500 nm to 700 nm, from 550 nm to 750 nm, from 600 nm to 800 nm, from 650 nm to 850 nm, from 700 nm to 900 nm, or from 10 nm to 900 nm in diameter. In some cases, a nanoparticle may be less than 1000 nm in diameter. In some cases, a particle may comprise a diameter of about 30 nm to about 800 nm. In some cases, a particle comprises a diameter of about 60 nm to about 600 nm. In some cases, a particle comprises a diameter of about 60 nm to about 500 nm. In some cases, a particle comprises a diameter of about 60 nm to about 400 nm. In some cases, a particle comprises a diameter of about 60 nm to about 300 nm. In some cases, a particle comprises a diameter of about 60 nm to about 200 nm. In some cases, a particle comprises a diameter of about 60 nm to about 150 nm. In some cases, a particle comprises a diameter of about 80 nm to about 500 nm. In some cases, a particle comprises a diameter of about 80 nm to about 400 nm. In some cases, a particle comprises a diameter of about 80 nm to about 300 nm. In some cases, a particle comprises a diameter of about 80 nm to about 200 nm. In some cases, a particle comprises a diameter of about 80 nm to about 150 nm. In some cases, a particle comprises a diameter of about 100 nm to about 500 nm. In some cases, a particle comprises a diameter of about 100 nm to about 400 nm. In some cases, a particle comprises a diameter of about 100 nm to about 300 nm. In some cases, a particle comprises a diameter of about 100 nm to about 200 nm. In some cases, a particle comprises a diameter of about 100 nm to about 150 nm. In some cases, a particle comprises a diameter of about 120 nm to about 600 nm. In some cases, a particle comprises a diameter of about 120 nm to about 500 nm. In some cases, a particle comprises a diameter of about 120 nm to about 400 nm. In some cases, a particle comprises a diameter of about 120 nm to about 350 nm. In some cases, a particle comprises a diameter of about 120 nm to about 300 nm. In some cases, a particle comprises a diameter of about 120 nm to about 200 nm. In some cases, a particle comprises a diameter of about 150 nm to about 600 nm. In some cases, a particle comprises a diameter of about 150 nm to about 500 nm. In some cases, a particle comprises a diameter of about 150 nm to about 400 nm. In some cases, a particle comprises a diameter of about 150 nm to about 300 nm. In some cases, a particle comprises a diameter of about 200 nm to about 400 nm. In some cases, a particle comprises a diameter of about 200 nm to about 600 nm. In some cases, a particle comprises a diameter of at least about 100 nm. In some cases, a particle comprises a diameter of at most 500 nm.

In some cases, a particle of the present disclosure may be a microparticle. A microparticle may be a particle that is from about 1 μm to about 1000 μm in diameter. For example, the microparticles disclosed here can be at least 1 μm, at least 10 μm, at least 100 μm, at least 200 μm, at least 300 μm, at least 400 μm, at least 500 μm, at least 600 μm, at least 700 μm, at least 800 μm, at least 900 μm, from 10 μm to 50 μm, from 50 μm to 100 μm, from 100 μm to 150 μm, from 150 μm to 200 μm, from 200 μm to 250 μm, from 250 μm to 300 μm, from 300 μm to 350 μm, from 350 μm to 400 μm, from 400 μm to 450 μm, from 450 μm to 500 μm, from 500 μm to 550 μm, from 550 μm to 600 μm, from 600 μm to 650 μm, from 650 μm to 700 μm, from 700 μm to 750 μm, from 750 μm to 800 μm, from 800 μm to 850 μm, from 850 μm to 900 μm, from 100 μm to 300 μm, from 150 μm to 350 μm, from 200 μm to 400 μm, from 250 μm to 450 μm, from 300 μm to 500 μm, from 350 μm to 550 μm, from 400 μm to 600 μm, from 450 μm to 650 μm, from 500 μm to 700 μm, from 550 μm to 750 μm, from 600 μm to 800 μm, from 650 μm to 850 μm, from 700 μm to 900 μm, or from 10 μm to 900 μm in diameter. In some cases, a microparticle may be less than 1000 μm in diameter. In some cases, a microparticle may comprise a diameter of about 1 μm to about 2 μm. In some cases, a microparticle may comprise a diameter of about 1 μm to about 1.5 μm.

A substrate (such as a particle) may comprise a degree of shape or size uniformity or non-uniformity. A physical measure of such heterogeneity may be polydispersity, which tracks size uniformity of a substrate, and may be defined as the square of the ratio of the standard deviation and the mean of substrate size (e.g., particle diameter). Alternatively, polydispersity may be a ratio of (1) weight average molecular weight to (2) number average molecular weight for a substrate (e.g., for a collection of particles), and therefore serves as a measure of mass variance for the substrate. A substrate may comprise a low polydispersity value, indicating a high degree of size uniformity. For example, a substrate (e.g., a collection of a substrate comprising a plurality of copies of the substrate) may comprise a polydispersity index of at most 1.6, at most 1.4, at most 1.2, at most 1, at most 0.8, at most 0.6, at most 0.5, at most 0.4, at most 0.3, at most 0.25, at most 0.2, at most 0.15, at most 0.1, at most 0.05, at most 0.03, or at most 0.02. Alternatively, a substrate may comprise a high polydispersity index, indicating a degree of size and/or mass variation. For example, a substrate (e.g., a collection of a substrate comprising a plurality of copies of the substrate) may comprise a polydispersity index of at least 0.3, at least 0.4, at least 0.5, at least 0.6, at least 0.8, at least 1, at least 1.2, at least 1.4, at least 1.6, at least 1.8, at least 2, at least 2.2, at least 2.5, or at least 3.

A particle may be substantially spherical. A particle may comprise an oblong geometry. A particle may comprise a surface feature, such as a well, a trench, or a substantially flat region.

A particle may be provided at a range of concentrations. A particle may be provided at a concentration of at least 10 pM. A particle may be provided at a concentration of at least 100 pM. A particle may be provided at a concentration of at least 1 nM. A particle may be provided at a concentration of at least 10 nM. A particle may be provided at a concentration of at most 100 nM. A particle may be provided at a concentration of at most 10 nM. A particle may be provided at a concentration of at most 1 nM. A particle may be provided at a concentration of at most 100 pM. A particle may be provided at a concentration of at most 10 pM. A particle may be provided at a concentration of at most 1 pM. A particle may be provided at a concentration between 100 fM and 100 nM. A particle may be provided at a concentration between 100 fM and 10 pM. A particle may be provided at a concentration between 1 pM and 100 pM. A particle may be provided at a concentration between 10 pM and 1 nM. A particle may be provided at a concentration between 100 pM and 10 nM. A particle may be provided at a concentration between 1 nM and 100 nM. A particle may be provided at a concentration of at least 10 ng/ml. A particle may be provided at a concentration of at least 100 ng/ml. A particle may be provided at a concentration of at least 1 μg/ml. A particle may be provided at a concentration of at least 10 μg/ml. A particle may be provided at a concentration of at least 100 μg/ml. A particle may be provided at a concentration of at least 1 mg/ml. A particle may be provided at a concentration of at least mg/ml. A particle may be provided at a concentration of at least 10 mg/ml. A particle may be provided at a concentration of at most 10 mg/ml. A particle may be provided at a concentration of at most 1/ml. A particle may be provided at a concentration of at most 100 μg/ml. A particle may be provided at a concentration of at most 10 μg/ml. A particle may be provided at a concentration of at most 1 μg/ml. A particle may be provided at a concentration of at most 100 ng/ml. A particle may be provided at a concentration of at most 10 ng/ml.

A particle may be contacted to a biological sample at a range of volume ratios. A solution comprising a particle may be combined with a biological sample, at a volume ratio of greater than about 100:1, about 100:1, about 80:1, about 60:1, about 50:1, about 40:1, about 30:1, about 25:1, about 20:1, about 15:1, about 12:1, about 10:1, about 8:1, about 6:1, about 5:1, about 4:1, about 3:1, about 5:2, about 2:1, about 3:2, about 1:1, about 2:3, about 1:2, about 2:5, about 1:3, about 1:4, about 1:5, about 1:6, about 1:8, about 1:10, about 1:12, about 1:15, about 1:20, about 1:25, about 1:30, about 1:40, about 1:50, about 1:60, about 1:80, about 1:100, or less than about 1:100.

In some cases, the ratio between surface area and mass can be a determinant of a particle's properties. In some cases, the number and types of biomolecules that a particle adsorbs from a solution varies with the particle's surface area to mass ratio. In some cases, a particle can have a surface area to mass ratios of 3 to 30 cm²/mg, 5 to 50 cm²/mg, 10 to 60 cm²/mg, 15 to 70 cm²/mg, 20 to 80 cm²/mg, 30 to 100 cm²/mg, 35 to 120 cm²/mg, 40 to 130 cm²/mg, 45 to 150 cm²/mg, 50 to 160 cm²/mg, 60 to 180 cm²/mg, 70 to 200 cm²/mg, 80 to 220 cm²/mg, 90 to 240 cm²/mg, 100 to 270 cm²/mg, 120 to 300 cm²/mg, 200 to 500 cm²/mg, 10 to 300 cm²/mg, 1 to 3000 cm²/mg, 20 to 150 cm²/mg, 25 to 120 cm²/mg, or from 40 to 85 cm²/mg. In some cases, small particles (e.g., with diameters of 50 nm or less) can have significantly higher surface area to mass ratios, stemming in part from the higher order dependence on diameter by mass than by surface area. In some cases (e.g., for small particles), the particles can have surface area to mass ratios of 200 to 1000 cm²/mg, 500 to 2000 cm²/mg, 1000 to 4000 cm²/mg, 2000 to 8000 cm²/mg, or 4000 to 10000 cm²/mg. In some cases (e.g., for large particles), the particles can have surface area to mass ratios of 1 to 3 cm²/mg, 0.5 to 2 cm²/mg, 0.25 to 1.5 cm²/mg, or 0.1 to 1 cm²/mg.

In some cases, a plurality of particles (e.g., of a particle panel) used with the methods described herein may have a range of surface area to mass ratios. In some cases, the range of surface area to mass ratios for a plurality of particles is less than 100 cm²/mg, 80 cm²/mg, 60 cm²/mg, 40 cm²/mg, 20 cm²/mg, 10 cm²/mg, 5 cm²/mg, or 2 cm²/mg. In some cases, the surface area to mass ratios for a plurality of particles varies by no more than 40%, 30%, 20%, 10%, 5%, 3%, 2%, or 1% between the particles in the plurality. In some cases, the plurality of particles may comprise at least 2, 3, 4, 5, 6, 7, 8, 9, 10, 12, 15, 20, or more different types of particles.

In some cases, a plurality of particles (e.g., in a particle panel) may comprise a range of surface area to mass ratios. In some cases, the range of surface area to mass ratios for a plurality of particles is greater than 100 cm²/mg, 150 cm²/mg, 200 cm²/mg, 250 cm²/mg, 300 cm²/mg, 400 cm²/mg, 500 cm²/mg, 800 cm²/mg, 1000 cm²/mg, 1200 cm²/mg, 1500 cm²/mg, 2000 cm²/mg, 3000 cm²/mg, 5000 cm²/mg, 6000 cm²/mg, 7500 cm²/mg, 10000 cm²/mg, or more. In some cases, the surface area to mass ratios for a plurality of particles (e.g., within a panel) can vary by more than 100%, 200%, 300%, 400%, 500%, 1000%, 10000% or more. In some cases, the plurality of particles with a wide range of surface area to mass ratios may comprise at least 2, 3, 4, 5, 6, 7, 8, 9, 10, 12, 15, 20, or more different types of particles.

A particle may comprise a wide range of physical properties. A physical property of a particle may comprise composition, size, surface charge, hydrophobicity, hydrophilicity, surface functionalization, surface topography, surface curvature, porosity, core material, shell material, shape, or any combination thereof.

A surface functionalization may comprise a polymerizable functional group, a positively or negatively charged functional group, a zwitterionic functional group, an acidic or basic functional group, a polar functional group, or any combination thereof. In some cases, a surface functionalization comprises a polar functional group, an acidic functional group, a basic functional group, a charged functional group, a polymerizable functional group, or any combination thereof. In some cases, a surface functionalization may comprise an aminopropyl functionalization, an amine functionalization, an amide functionalization, a boronic acid functionalization, a carboxylic acid functionalization, a methyl functionalization, an N-succinimidyl ester functionalization, a PEG functionalization, a streptavidin functionalization, a methyl ether functionalization, a triethoxylpropylaminosilane functionalization, a thiol functionalization, a PCP functionalization, a citrate functionalization, a lipoic acid functionalization, a BPEI functionalization, carboxyl functionalization, a hydroxyl functionalization, or any combination thereof. In some cases, a surface functionalization may comprise carboxyl groups, hydroxyl groups, thiol groups, cyano groups, nitro groups, ammonium groups, alkyl groups, imidazolium groups, sulfonium groups, pyridinium groups, pyrrolidinium groups, phosphonium groups, aminopropyl groups, amine groups, amide groups, boronic acid groups, N-succinimidyl ester groups, PEG groups, streptavidin, methyl ether groups, triethoxylpropylaminosilane groups, PCP groups, citrate groups, lipoic acid groups, BPEI groups, or any combination thereof. In some cases, a surface functionalization may be present at various ranges of densities on a particle. In some cases, a surface functionalization comprises an average density of at least about 1 functional group per 20 nm² on a surface of a particle. In some cases, a surface functionalization may comprise an average density of at least about 1 functional group per 30 nm² on a surface of a particle. In some cases, a surface functionalization may comprise an average density of at least about 1 functional group per 40 nm² on a surface of a particle. In some cases, a surface functionalization may comprise an average density of at least about 1 functional group per 50 nm² on a surface of a particle. In some cases, a surface functionalization may comprise an average density of at least about 1 functional group per 60 nm² on a surface of a particle. In some cases, a surface functionalization may comprise an average density of at least about 1 functional group per 80 nm² on a surface of a particle. In some cases, a surface functionalization may comprise an average density of at most about 1 functional group per 80 nm² on a surface of a particle. In some cases, a surface functionalization may comprise an average density of at most about 1 functional group per 60 nm² on a surface of a particle. In some cases, a surface functionalization may comprise an average density of at most about 1 functional group per 50 nm² on a surface of a particle. In some cases, a surface functionalization may comprise an average density of at most about 1 functional group per 40 nm² on a surface of a particle. In some cases, a surface functionalization may comprise an average density of at most about 1 functional group per 30 nm² on a surface of a particle. In some cases, a surface functionalization may comprise an average density of at most about 1 functional group per 20 nm² on a surface of a particle. In some cases, a surface functionalization may comprise an average density about 1 functional group per 20 nm² to at most about 1 functional group per 60 nm² on a surface of a particle.

In some cases, a particle may be selected from the group consisting of: micelles, liposomes, iron oxide particles, silver particles, gold particles, palladium particles, quantum dots, platinum particles, titanium particles, silica particles, metal or inorganic oxide particles, synthetic polymer particles, copolymer particles, terpolymer particles, polymeric particles with metal cores, polymeric particles with metal oxide cores, polystyrene sulfonate particles, polyethylene oxide particles, polyoxyethylene glycol particles, polyethylene imine particles, polylactic acid particles, polycaprolactone particles, polyglycolic acid particles, poly(lactide-co-glycolide polymer particles, cellulose ether polymer particles, polyvinylpyrrolidone particles, polyvinyl acetate particles, polyvinylpyrrolidone-vinyl acetate copolymer particles, polyvinyl alcohol particles, acrylate particles, polyacrylic acid particles, crotonic acid copolymer particles, polyethlene phosphonate particles, polyalkylene particles, carboxy vinyl polymer particles, sodium alginate particles, carrageenan particles, xanthan gum particles, gum acacia particles, Arabic gum particles, guar gum particles, pullulan particles, agar particles, chitin particles, chitosan particles, pectin particles, karaya tum particles, locust bean gum particles, maltodextrin particles, amylose particles, corn starch particles, potato starch particles, rice starch particles, tapioca starch particles, pea starch particles, sweet potato starch particles, barley starch particles, wheat starch particles, hydroxypropylated high amylose starch particles, dextrin particles, levan particles, elsinan particles, gluten particles, collagen particles, whey protein isolate particles, casein particles, milk protein particles, soy protein particles, keratin particles, polyethylene particles, polycarbonate particles, polyanhydride particles, polyhydroxyacid particles, polypropylfumerate particles, polycaprolactone particles, polyamine particles, polyacetal particles, polyether particles, polyester particles, poly(orthoester) particles, polycyanoacrylate particles, polyurethane particles, polyphosphazene particles, polyacrylate particles, polymethacrylate particles, polycyanoacrylate particles, polyurea particles, polyamine particles, polystyrene particles, poly(lysine) particles, chitosan particles, dextran particles, poly(acrylamide) particles, derivatized poly(acrylamide) particles, gelatin particles, starch particles, chitosan particles, dextran particles, gelatin particles, starch particles, poly-β-amino-ester particles, poly(amido amine) particles, poly lactic-co-glycolic acid particles, polyanhydride particles, bioreducible polymer particles, 2-(3-aminopropylamino) ethanol particles, and any combination thereof.

In some cases, particles of the present disclosure may differ by one or more physicochemical property. The one or more physicochemical property is selected from the group consisting of: composition, size, surface charge, hydrophobicity, hydrophilicity, roughness, density surface functionalization, surface topography, surface curvature, porosity, core material, shell material, shape, and any combination thereof. The surface functionalization may comprise a macromolecular functionalization, a small molecule functionalization, or any combination thereof. A small molecule functionalization may comprise an aminopropyl functionalization, amine functionalization, an amide functionalization, boronic acid functionalization, carboxylic acid functionalization, alkyl group functionalization, N-succinimidyl ester functionalization, monosaccharide functionalization, phosphate sugar functionalization, sulfurylated sugar functionalization, ethylene glycol functionalization, streptavidin functionalization, methyl ether functionalization, trimethoxysilylpropyl functionalization, silica functionalization, triethoxylpropylaminosilane functionalization, thiol functionalization, PCP functionalization, citrate functionalization, lipoic acid functionalization, ethyleneimine functionalization. A particle panel may comprise a plurality of particles with a plurality of small molecule functionalizations selected from the group consisting of silica functionalization, trimethoxysilylpropyl functionalization, dimethylamino propyl functionalization, phosphate sugar functionalization, amine functionalization, and carboxyl functionalization.

A small molecule functionalization may comprise a polar functional group. Non-limiting examples of polar functional groups comprise carboxyl group, a hydroxyl group, a thiol group, a cyano group, a nitro group, an ammonium group, an imidazolium group, a sulfonium group, a pyridinium group, a pyrrolidinium group, a phosphonium group or any combination thereof. In some cases, the functional group is an acidic functional group (e.g., sulfonic acid group, carboxyl group, and the like), a basic functional group (e.g., amino group, cyclic secondary amino group (such as pyrrolidyl group and piperidyl group), pyridyl group, imidazole group, guanidine group, etc.), a carbamoyl group, a hydroxyl group, an aldehyde group and the like.

A small molecule functionalization may comprise an ionic or ionizable functional group. Non-limiting examples of ionic or ionizable functional groups comprise an ammonium group, an imidazolium group, a sulfonium group, a pyridinium group, a pyrrolidinium group, a phosphonium group.

A small molecule functionalization may comprise a polymerizable functional group. Non-limiting examples of the polymerizable functional group include a vinyl group and a (meth)acrylic group. In some cases, the functional group is pyrrolidyl acrylate, acrylic acid, methacrylic acid, acrylamide, 2-(dimethylamino)ethyl methacrylate, hydroxyethyl methacrylate and the like.

A surface functionalization may comprise a charge. For example, a particle can be functionalized to carry a net neutral surface charge, a net positive surface charge, a net negative surface charge, or a zwitterionic surface. A zwitterionic particle surface may be zwitterionic over at least 1, at least 2, at least 3, at least 4, at least 5, at least 6 or more pH units. Surface charge can be a determinant of the types of biomolecules collected on a particle. Accordingly, optimizing a particle panel may comprise selecting particles with different surface charges, which may not only increase the number of different proteins collected on a particle panel, but also increase the likelihood of identifying a biological state of a sample. A particle panel may comprise a positively charged particle and a negatively charged particle. A particle panel may comprise a positively charged particle and a neutral particle. A particle panel may comprise a positively charged particle and a zwitterionic particle. A particle panel may comprise a neutral particle and a negatively charged particle. A particle panel may comprise a neutral particle and a zwitterionic particle. A particle panel may comprise a negative particle and a zwitterionic particle. A particle panel may comprise a positively charged particle, a negatively charged particle, and a neutral particle. A particle panel may comprise a positively charged particle, a negatively charged particle, and a zwitterionic particle. A particle panel may comprise a positively charged particle, a neutral particle, and a zwitterionic particle. A particle panel may comprise a negatively charged particle, a neutral particle, and a zwitterionic particle. In some cases, a charge of a particle may be determined by measuring the zeta potential of the particle.

### Particle Panels

The present disclosure provides compositions and methods of use thereof for assaying a sample for proteins. Compositions described herein may include particle panels comprising one or more than one distinct particle types. Particle panels described herein can vary in the number of particle types and the diversity of particle types in a single panel. For example, particles in a panel may vary based on size, polydispersity, shape and morphology, surface charge, surface chemistry and functionalization, and base material. Panels may be incubated with a sample to be analyzed for protein composition. Proteins in the sample may adsorb to the surface of the different particle types in the particle panel to form a protein corona. The types of proteins which adsorb to a certain particle type in the particle panel may depend on the composition, size, and surface charge of the particle type. Thus, each particle type in a panel may have different protein coronas due to adsorbing a different set of proteins, different concentrations of a particular protein, or a combination thereof. Each particle type in a panel may have mutually exclusive protein coronas or may have overlapping protein coronas. Overlapping protein coronas can overlap in protein identity, in protein concentration, or both.

The present disclosure also provides methods for selecting a particle types for inclusion in a panel depending on the sample type. Particle types included in a panel may be a combination of particles that are optimized for removal of highly abundant proteins. Particle types also consistent for inclusion in a panel are those selected for adsorbing particular proteins of interest. In some cases, the particles may be nanoparticles. In some cases, the particles may be microparticles. In some cases, the particles may be a combination of nanoparticles and microparticles.

A particle panel including any number of distinct particle types disclosed herein, may enrich and identify a single protein or protein group. In some cases, the single protein or protein group may comprise proteins having different post-translational modifications. For example, a first particle type in the particle panel may enrich a protein or protein group having a first post-translational modification, a second particle type in the particle panel may enrich the same protein or same protein group having a second post-translational modification, and a third particle type in the particle panel may enrich the same protein or same protein group lacking a post-translational modification. In some cases, the particle panel including any number of distinct particle types disclosed herein, may enrich and identify a single protein or protein group by binding different domains, sequences, or epitopes of the single protein or protein group. For example, a first particle type in the particle panel may enrich a protein or protein group by binding to a first domain of the protein or protein group, and a second particle type in the particle panel may enrich the same protein or same protein group by binding to a second domain of the protein or protein group.

A particle panel may comprise a combination of particles with silica and polymer surfaces. For example, a particle panel may comprise a SPION coated with a thin layer of silica, a SPION coated with poly(dimethyl aminopropyl methacrylamide) (PDMAPMA), and a SPION coated with poly(ethylene glycol) (PEG). A particle panel of the present disclosure can also comprise two or more particles selected from the group consisting of silica coated SPION, an N-(3-Trimethoxysilylpropyl) diethylenetriamine coated SPION, a PDMAPMA coated SPION, a carboxyl-functionalized polyacrylic acid coated SPION, an amino surface functionalized SPION, a polystyrene carboxyl functionalized SPION, a silica particle, and a dextran coated SPION. A particle panel of the present disclosure may also comprise two or more particles selected from the group consisting of a surfactant free carboxylate microparticle, a carboxyl functionalized polystyrene particle, a silica coated particle, a silica particle, a dextran coated particle, an oleic acid coated particle, a boronated nanopowder coated particle, a PDMAPMA coated particle, a Poly(glycidyl methacrylate-benzylamine) coated particle, and a Poly(N-[3-(Dimethylamino) propyl]methacrylamide-co-[2-(methacryloyloxy)ethyl]dimethyl-(3-sulfopropyl) ammonium hydroxide, P(DMAPMA-co-SBMA) coated particle. A particle panel of the present disclosure may comprise silica-coated particles, N-(3-Trimethoxysilylpropyl) diethylenetriamine coated particles, poly(N-(3-(dimethylamino) propyl) methacrylamide) (PDMAPMA)-coated particles, phosphate-sugar functionalized polystyrene particles, amine functionalized polystyrene particles, polystyrene carboxyl functionalized particles, ubiquitin functionalized polystyrene particles, dextran coated particles, or any combination thereof.

A particle panel of the present disclosure may comprise a silica functionalized particle, an amine functionalized particle, a silicon alkoxide functionalized particle, a carboxylate functionalized particle, and a benzyl or phenyl functionalized particle. A particle panel of the present disclosure may comprise a silica functionalized particle, an amine functionalized particle, a silicon alkoxide functionalized particle, a polystyrene functionalized particle, and a saccharide functionalized particle. A particle panel of the present disclosure may comprise a silica functionalized particle, an N-(3-Trimethoxysilylpropyl) diethylenetriamine functionalized particle, a PDMAPMA functionalized particle, a dextran functionalized particle, and a polystyrene carboxyl functionalized particle. A particle panel of the present disclosure may comprise 5 particles including a silica functionalized particle, an amine functionalized particle, a silicon alkoxide functionalized particle.

A particle panel of the present disclosure may comprise a silica particle, an amine functionalized particle, and a polyethylene glycol-functionalized particle. The particle panel may further comprise a carboxylate functionalized particle, such as a carboxylate functionalized styrene particle. The particle panel may further comprise a saccharide-coated particle. In some cases, the saccharide-coated particle is a dextran-coated particle. The particle panel may further comprise a sulfuryl functionalized particle. The sulfuryl functionalized particle may comprise a positively charged surface functionalization such as an amine, and thereby may be zwitterionic. The particle panel may further comprise a particle with a boronated or boronic acid functionalized surface. The particle panel may further comprise a particle with an oleic acid functionalized surface. The particle panel may comprise at least one microparticle.

The present disclosure includes compositions (e.g., particle panels) and methods that comprise two or more particles differing in at least one physicochemical property. A composition or method of the present disclosure may comprise 3 to 6 particles differing in at least one physicochemical property. A composition or method of the present disclosure may comprise 4 to 8 particles differing in at least one physicochemical property. A composition or method of the present disclosure may comprise 4 to 10 particles differing in at least one physicochemical property. A composition or method of the present disclosure may comprise 5 to 12 particles differing in at least one physicochemical property. A composition or method of the present disclosure may comprise 6 to 14 particles differing in at least one physicochemical property. A composition or method of the present disclosure may comprise 8 to 15 particles differing in at least one physicochemical property. A composition or method of the present disclosure may comprise 10 to 20 particles differing in at least one physicochemical property. A composition or method of the present disclosure may comprise at least 2 distinct particle types, at least 3 distinct particle types, at least 4 distinct particle types, at least 5 distinct particle types, at least 6 distinct particle types, at least 7 distinct particle types, at least 8 distinct particle types, at least 9 distinct particle types, at least 10 distinct particle types, at least 11 distinct particle types, at least 12 distinct particle types, at least 13 distinct particle types, at least 14 distinct particle types, at least 15 distinct particle types, at least 20 distinct particle types, at least 25 particle types, or at least 30 distinct particle types.

An example of a particle panel of the present disclosure is summarized in TABLE 3, and particles span sizes of about 200 nm to about 400 nm, zeta potentials of about −40 mV to about 30 mV, pKa values of about 4.5 to about 11.78, Log P (log of partition coefficient) values of about −4.2 to about 0.7, relative PGs (ratio of the number of detected protein groups relative to the number of protein groups detected by SP-003) values of about 0.8 to about 1.3, and peptide mass (collected from the particles before mass spectrometry) values of about less than about 2 μg to greater than about 3 μg.

Another example of a particle panel of the present disclosure comprises 2, 3, 4, 5, 6, or 7 particles from the group SP-003, SP-006, SP-007, SP-118, SP-128, SP-229, and SP-251. For example, the particle panel summarized in TABLE 2 comprises SP-003, SP-007, SP-118, SP-128, and SP-229. In some instances, the particles (e.g., such as those in TABLE 2) span sizes of 220 nm to 400 nm, zeta potentials of −55.3 mV to 40 mV, pKa values of 4.6 to 12, log P of about −5 to about 0.7, relative PGs of about 1 to about 1.2, and peptide mass greater than 1 or greater than 3 μg.

In some cases, a particle panel may comprise a particle listed in TABLE 2, below. A particle panel may comprise at least two particles listed in TABLE 2. In some cases, a particle panel may comprise at least three particles listed in TABLE 2. In some cases, a particle panel may comprise at least four particles listed in TABLE 2. In some cases, a particle panel may comprise the particles listed in TABLE 2.

TABLE 2
Example of a particle panel of the present disclosure

| Particle | |
|---|---|
| Name | Description |
| SP-003 | Silica-Coated SPION |
| SP-007 | Poly(dimethylaminopropylmethacrylamide)-coated SPION |
| SP-118 | Glucose-6-phosphate functionalized SPION |
| SP-128 | Mixed amide, carboxylate functionalized, silica-coated SPION |
| SP-229 | N¹-(3-(trimethoxysilyl)propyl)hexane-1,6-diamine functionalized, silica-coated SPION |

In some cases, a particle panel may comprise a particle listed in TABLE 3, below. In some cases, a particle panel may comprise at least two particles listed in TABLE 3. In some cases, a particle panel may comprise at least three particles listed in TABLE 3. In some cases, a particle panel may comprise at least four particles listed in TABLE 3. In some cases, a particle panel may comprise the particles listed in TABLE 3.

TABLE 3
Example of a particle panel of the present disclosure

| Particle | |
|---|---|
| Name | Description |
| SP-003 | Silica-Coated SPION |
| SP-006 | N-(3-Trimethoxysilylpropyl)diethylenetriamine-coated SPION |
| SP-007 | Poly(dimethylaminopropylmethacrylamide)-coated SPION |
| SP-339 | Carboxyl functionalized polystyrene-coated SPION |
| SP-373 | Dextran-coated SPION |

In some cases, a particle panel may comprise a particle listed in TABLE 4, below. In some cases, a particle panel may comprise at least two particles listed in TABLE 4. In some cases, a particle panel may comprise at least three particles listed in TABLE 4. In some cases, a particle panel may comprise at least four particles listed in TABLE 4. In some cases, a particle panel may comprise the particles listed in TABLE 4.

TABLE 4
Example of a particle panel of the present disclosure

| Particle | |
|---|---|
| ID | Description |
| SP-339 | Polystyrene particles, Paramagnetic, Carboxyl-functionalized (PS-MAG-COOH) |
| SP-373 | Magnetizable Nanoparticles and magnetizable microparticles, Dextran |
| | based//plain/25 mg/ml |
| SP-003 | Superparamagnetic, silica coated |
| SP-006 | Silica coated, amine |
| SP-007 | PDMAPMA coated (Dimethylamine) |

In some cases, a particle panel may comprise a particle listed in TABLE 5, below. In some cases, a particle panel may comprise at least two particles listed in TABLE 5. In some cases, a particle panel may comprise at least three particles listed in TABLE 5. In some cases, a particle panel may comprise at least four particles listed in TABLE 5. In some cases, a particle panel may comprise the particles listed in TABLE 5.

TABLE 5
Example of a particle panel of the present disclosure

| Particle | |
|---|---|
| ID | Description |
| SP-333 | Carboxylate |
| SP-347 | Silica |
| SP-353 | Amino |
| SP-389 | Wheat Germ Agglutinin |
| SP-008 | 1,2,4,5-Benzenetetracarboxylic acid coated SPION |

In some cases, a particle panel of the present disclosure may comprise at least one, at least two, at least 3, at least 4, or at least 5 particles, each particle selected from the group consisting of a superparamagnetic iron oxide particle (SPION) comprising a silica surface, a SPION comprising an N-(3-Trimethoxysilylpropyl) diethylenetriamine surface, a SPION comprising a Poly(dimethyl aminopropyl methacrylamide) (Dimethylamine) surface, a SPION comprising a carboxyl functionalized polystyrene surface, and a SPION comprising a dextran coating. In some cases, a particle panel of the present disclosure may comprise a SPION comprising a poly(N-(3-(dimethylamino) propyl) methacrylamide) (PDMAPMA) surface. In some cases, a particle panel of the present disclosure may comprise a SPION comprising a poly(oligo (ethylene glycol) methyl ether methacrylate) (POEGMA) surface. In some cases, a particle panel of the present disclosure may comprise a SPION comprising an N-(3-Trimethoxysilylpropyl) diethylenetriamine surface. In some cases, a particle panel of the present disclosure may comprise a SPION comprising a Poly(dimethyl aminopropyl methacrylamide) (Dimethylamine) surface. In some cases, a particle panel of the present disclosure may comprise a SPION comprising a dextran surface. In some cases, a particle panel of the present disclosure may comprise a SPION comprising a surface with a mixed chemistry based on amine-epoxy chemistry. In some cases, a particle panel of the present disclosure may comprise a SPION comprising a Polyzwitterion coated (Poly(N-[3-(Dimethylamino)propyl]methacrylamide-co-[2-(methacryloyloxy)ethyl]dimethyl-(3-sulfopropyl)ammonium hydroxide, P(DMAPMA-co-SBMA)) surface. In some cases, a particle panel of the present disclosure may comprise a SPION comprising styrene surface comprising an oleic acid functionalization. In some cases, a particle panel of the present disclosure may comprise a SPION comprising a boronated styrene surface. In some cases, a particle panel of the present disclosure may comprise a SPION comprising a carboxylated styrene surface. In some cases, a particle panel of the present disclosure may comprise a SPION comprising a carboxylated styrene surface. In some cases, a particle panel of the present disclosure may comprise a SPION comprising a strongly acidic silica surface. A particle panel of the present disclosure may comprise at least one particle, at least 2 particles, at least 3 particles, or at least 4 particles, or at least 5 particles, each selected from the group consisting of a silica-coated SPION, a poly(dimethylaminopropylmethacrylamide)-coated SPION, an N-(3-Trimethoxysilylpropyl) diethylenetriamine-coated SPION, a 1,6-hexanediamine-coated SPION, and an N1-(3-(trimethoxysilyl) propyl) hexane-1,6-diamine functionalized, silica-coated SPION. A particle panel of the present disclosure may comprise a silica-coated SPION, a poly(dimethylaminopropylmethacrylamide)-coated SPION, an N-(3-Trimethoxysilylpropyl) diethylenetriamine-coated SPION, a 1,6-hexanediamine-coated SPION, and an N1-(3-(trimethoxysilyl) propyl) hexane-1,6-diamine functionalized, silica-coated SPION.

In some cases, particles of the present disclosure may be used to serially interrogate a sample by incubating a first particle type with the sample to form a biomolecule corona on the first particle type, separating the first particle type, incubating a second particle type with the sample to form a biomolecule corona on the second particle type, separating the second particle type, and repeating the interrogating (by incubation with the sample) and the separating for any number of particle types. In some cases, the biomolecule corona on each particle type used for serial interrogation of a sample may be analyzed by protein corona analysis. The biomolecule content of the supernatant may be analyzed following serial interrogation with one or more particle types.

### Protein Groups

The particle panels disclosed herein can be used to identify a number of proteins, peptides, or protein groups using a method disclosed herein. Feature intensities, as disclosed herein, may refer to the intensity of a discrete spike (“feature”) seen on a plot of mass to charge ratio versus intensity from a mass spectrometry run of a sample. These features can correspond to variably ionized fragments of peptides and/or proteins. Feature intensities can be sorted into protein groups. Protein groups refer to two or more proteins that are identified by a shared peptide sequence. Alternatively, a protein group can refer to one protein that is identified using a unique identifying sequence. For example, if in a sample, a peptide sequence is assayed that is shared between two proteins (Protein 1: XYZZX and Protein 2: XYZYZ), a protein group could be the “XYZ protein group” having two members (protein 1 and protein 2). Alternatively, if the peptide sequence is unique to a single protein (Protein 1), a protein group could be the “ZZX” protein group having one member (Protein 1). Each protein group can be supported by more than one peptide sequence. Protein detected or identified according to the instant disclosure can refer to a distinct protein detected in the sample (e.g., distinct relative other proteins detected using mass spectrometry). Thus, analysis of proteins present in distinct coronas corresponding to the distinct particle types in a particle panel, yields a high number of feature intensities. This number decreases as feature intensities are processed into distinct peptides, further decreases as distinct peptides are processed into distinct proteins, and further decreases as peptides are grouped into protein groups (two or more proteins that share a distinct peptide sequence),

### Biomolecule Coronas

Aspects of the present disclosure provide compositions, systems, and methods for collecting biomolecules on particles (e.g., nanoparticles and microparticles) (as well as other types of sensor elements such as polymer matrices, filters, rods, and extended surfaces). In some cases, a particle may adsorb a plurality of biomolecules upon contact with a biological sample, thereby forming a biomolecule corona on the surfaces of the particles. In some cases, the biomolecule corona may comprise proteins, lipids, nucleic acids, metabolites, saccharides, small molecules (e.g., sterols), and other biological species present in a sample. In some cases, a biomolecule corona comprising proteins may also be referred to as a ‘protein corona’, and may refer to all constituents adsorbed to a particle (e.g., proteins, lipids, nucleic acids, and other biomolecules), or may refer only to proteins adsorbed to the particle.

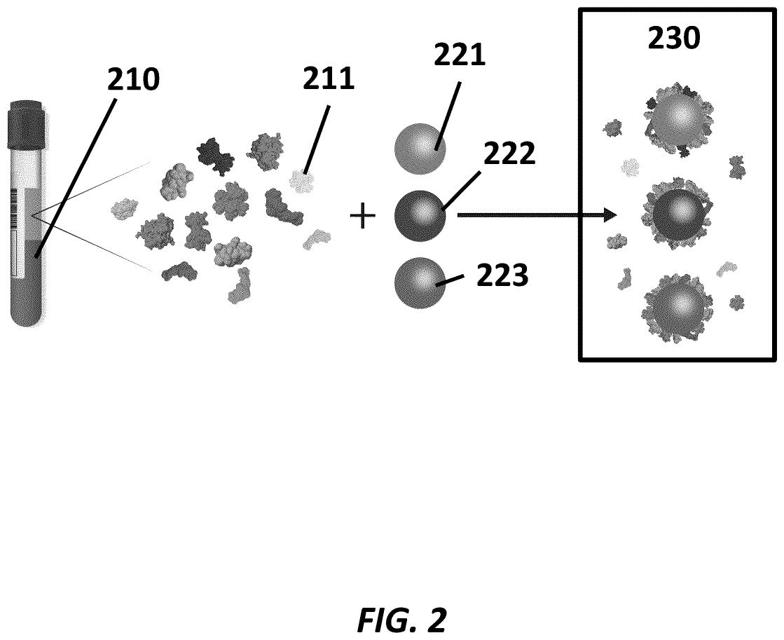

FIG. 2 provides a schematic overview of biomolecule formation, wherein a plurality of particles 221, 222, & 223 are contacted with a biological sample 210 comprising biomolecules molecules 211, and wherein each particle adsorbs a plurality of biomolecules from the biological sample to its surface 230. The different particles may be distinct particle types (depicted in the center of the figure, with the top, middle, and bottom spheres representing the three distinct particle types), such that each particle differs from the other particles by at least one physicochemical property. This difference in physicochemical properties can lead to the formation of different protein corona compositions on the particle surfaces.

The composition of the biomolecule corona may depend on a property of the particle. In many cases, the composition of the biomolecule corona is strongly dependent on the surface of the particle. Characteristics such as particle surface material (e.g., ceramic, polymer, metal, metal oxide, graphite, silicon dioxide, etc.), surface texture (rough, smooth, grooved, etc.), surface functionalization (e.g., carboxylate functionalized, amine functionalized, small molecule (e.g., saccharide) functionalized, etc.), shape, curvature, and size can each independently serve as determinants for biomolecule corona composition. In addition to surface features, the particle core composition, particle density, and particle surface area to mass ratio may each influence biomolecule corona composition. For example, two particles comprising the same surfaces and different cores may form different biomolecule coronas upon contact with the same sample.

Biomolecule corona formation may also be influenced by sample composition. For example, a first sample condition (e.g., low salinity) might favor the solubility of a particular analyte (e.g., an isoform of Bone Morphogenic Protein 1 (BMP1)), and thereby disfavor its binding in a biomolecule corona, while a second sample condition (e.g., high salinity) may diminish the solubility of the analyte, thereby driving its incorporation into a biomolecule corona.

Biomolecule corona composition may also depend on molecular level interactions between the biomolecules, themselves. An energetically favorable interaction between two biomolecules may promote their co-incorporation into a biomolecule corona. For example, if a first protein adsorbed to a particle comprises an affinity for a second protein in solution, the first protein may bind to a portion of the second protein, thereby driving its binding to the particle or to other proteins of the biomolecule corona of the particle. Analogously, a first biomolecule disposed within a biomolecule corona may comprise an energetically unfavorable interaction with a second biomolecule in a biological sample, thereby disfavoring its incorporation into a biomolecule corona. In part owing to these inter-biomolecule dependencies, biomolecule coronas provide sensitive platforms for directly and indirectly sensing biomolecules from a biological sample.

### Protein Analysis Methods

Biomolecules collected on a particle may be subjected to further analysis. A method may comprise collecting a biomolecule corona or a subset of biomolecules from a biomolecule corona. The collected biomolecule corona or the collected subset of biomolecules from the biomolecule corona may be subjected to further particle-based analysis (e.g., particle adsorption). The collected biomolecule corona or the collected subset of biomolecules from the biomolecule corona may be purified or fractionated (e.g., by a chromatographic method). The collected biomolecule corona or the collected subset of biomolecules from the biomolecule corona may be analyzed (e.g., by mass spectrometry). Furthermore, as biomolecule corona composition is dependent on solution-phase and particle-bound biomolecules as well as sample conditions (e.g., pH, osmolarity, lipid concentration), biomolecule corona composition can provide a sensitive measure of biomolecules which are not bound to a particle and of sample conditions.

The particles and methods of use thereof disclosed herein can bind a large number of unique biomolecules (e.g., proteins) in a biological sample (e.g., a biofluid). For example, a particle or particle panel disclosed herein can be incubated with a biological sample to form a protein corona comprising at least 5 protein groups, at least 10 protein groups, at least 15 protein groups, at least 20 protein groups, at least 25 protein groups, at least 50 protein groups, at least 80 protein groups, at least 100 protein groups, least 150 protein groups, at least 180 protein groups, at least 200 protein groups, at least 250 protein groups, at least 300 protein groups, at least 350 protein groups, at least 400 protein groups, at least 450 protein groups, at least 500 protein groups, at least 600 protein groups, at least 700 protein groups, at least 800 protein groups, at least 900 protein groups, at least 1000 protein groups, at least 1100 protein groups, at least 1200 protein groups, at least 1300 protein groups, at least 1400 protein groups, at least 1500 protein groups, at least 1600 protein groups, at least 1800 protein groups, at least 2000 protein groups, at least 2500, at least 5000 protein groups, at least 10000 protein groups, at least 15000 protein groups, at least 20000 protein groups, at least 25000 protein groups, at least 30000 protein groups, at least 35000 protein groups, at least 45000 protein groups, at least 50000 protein groups, at least 60000 protein groups, at least 70000 protein groups, at least 80000 protein groups, at least 90000 protein groups, or at least 100000 protein groups. A particle or particle panel disclosed herein can be incubated with a biological sample to form a protein corona comprising at most 5 protein groups, at most 10 protein groups, at most 20 protein groups, at most 30 protein groups, at most 40 protein groups, at most 50 protein groups, at most 60 protein groups, at most 80 protein groups, at most 100 protein groups, at most 150 protein groups, at most 200 protein groups, at most 250 protein groups, at most 300 protein groups, at most 400 protein groups, at most 500 protein groups, at most 600 protein groups, at most 800 protein groups, at most 1000 protein groups, at most 1200 protein groups, at most 1500 protein groups, at most 1800 protein groups, at most 2000 protein groups, at most 2500 protein groups, at most 3000 protein groups, at most 4000 protein groups, at most 5000 protein groups, at most 7500 protein groups, at most 10000 protein groups, at most 15000 protein groups, at most 20000 protein groups, at most 25000 protein groups, at most 50000 protein groups, at most 75000 protein groups, or at most 100000 protein groups. A particle disclosed herein can be incubated with a biological sample to form a protein corona comprising from 5 to 2500 protein groups. A particle or particle panel disclosed herein can be incubated with a biological sample to form a protein corona comprising from 5 to 50 protein groups. A particle disclosed herein can be incubated with a biological sample to form a protein corona comprising from 10 to 100 protein groups. A particle or particle panel disclosed herein can be incubated with a biological sample to form a protein corona comprising from 20 to 100 protein groups. A particle disclosed herein can be incubated with a biological sample to form a protein corona comprising from 20 to 400 protein groups. A particle or particle panel disclosed herein can be incubated with a biological sample to form a protein corona comprising from 50 to 500 protein groups. A particle disclosed herein can be incubated with a biological sample to form a protein corona comprising from 100 to 800 protein groups. A particle or particle panel disclosed herein can be incubated with a biological sample to form a protein corona comprising from 200 to 1000 protein groups. A particle or particle panel disclosed herein can be incubated with a biological sample to form a protein corona comprising from 300 to 1200 protein groups. A particle or particle panel disclosed herein can be incubated with a biological sample to form a protein corona comprising from 400 to 1500 protein groups. A particle or particle panel disclosed herein can be incubated with a biological sample to form a protein corona comprising from 500 to 2000 protein groups. A particle or particle panel disclosed herein can be incubated with a biological sample to form a protein corona comprising from 800 to 2500 protein groups. A particle or particle panel disclosed herein can be incubated with a biological sample to form a protein corona comprising from 1000 to 3000 protein groups. A particle or particle panel disclosed herein can be incubated with a biological sample to form a protein corona comprising from 1000 to 5000 protein groups. A particle or particle panel disclosed herein can be incubated with a biological sample to form a protein corona comprising from 2000 to 10000 protein groups. A particle or particle panel disclosed herein can be incubated with a biological sample to form a protein corona comprising from 5000 to 25000 protein groups. In some cases, several different types of particles can be used, separately or in combination, to identify large numbers of proteins in a particular biological sample. In other words, particles can be multiplexed in order to bind and identify large numbers of proteins in a biological sample. Protein corona analysis may compress the dynamic range of the analysis compared to a protein analysis of the original sample.

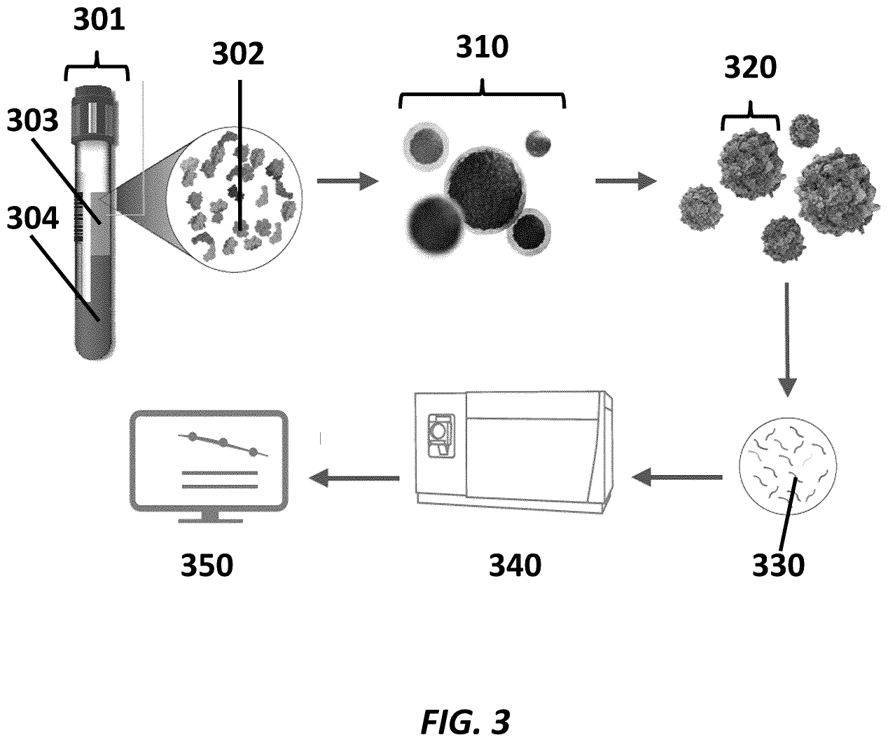

FIG. 3 provides an example of a particle-based biomolecule corona (e.g., protein corona) assay of the present disclosure. A biological sample (e.g., human plasma) 301 comprising a plurality of biomolecules 302 may be contacted to a plurality of particles 310. The sample may be treated, diluted, or split into a plurality of fractions 303 and 304 prior to analysis. For example, a whole blood sample may be fractionated into plasma and erythrocyte portions. Upon contact with the particles, a subset or the entirety of the plurality of biomolecules may adsorb to the particles, thereby forming biomolecule coronas 320 bound to the surfaces of the particles. Unbound biomolecules may be separated from the biomolecule coronas (e.g., through wash steps). The biomolecule coronas, or subsets thereof, may be collected from the particles. Alternatively, biomolecules of the biomolecule coronas may be fragmented or chemically treated while bound to the particles. In some assays, biomolecules (e.g., proteins) are fragmented (e.g., digested) while disposed in the biomolecule coronas to yield biomolecule (e.g., peptide) fragments 330. Biomolecules (or their chemically treated or fragmented derivatives) may be analyzed 340, for example by mass spectrometry, to yield data 350 representative of biomolecules 302 from the biological sample 301. The data may be analyzed to identify a biological state of the biological sample.

In some embodiments, an example of a biomolecule corona (e.g., protein corona) analysis workflow of the present disclosure which includes: particle incubation with a biological sample (e.g., plasma) under conditions suitable for adsorption of biomolecules from the biological sample to the particles to form biomolecule coronas; partitioning of the particle-plasma sample mixture into a plurality of partitions (e.g., wells on a multi-well plate); particle collection (e.g., with a magnet); a wash step or plurality of wash steps to remove analytes not adsorbed to the particles; resuspension of the particles and the biomolecules adsorbed thereto; biomolecule corona digestion or chemical treatment (e.g., protein reduction and digestion); and analysis of the biomolecule coronas or of biomolecules derived therefrom (e.g., by liquid chromatography-mass spectrometry (LC-MS) analysis). While this example provides parallel analyses across multiple wells of a multi-well plate, a method may comprise a single sample volume or a plurality of sample volumes, for example 2 volumes, 3 volumes, 4 volumes, 5 volumes, 6 volumes, 7 volumes, 8 volumes, 9 volumes, 10 volumes, 11 volumes, 12 volumes, 15 volumes, 18 volumes, 20 volumes, 22 volumes, 24 volumes, 25 volumes, 28 volumes, 30 volumes, 36 volumes, 40 volumes, 48 volumes, 50 volumes, 60 volumes, 70 volumes, 80 volumes, 90 volumes, 96 volumes, 128 volumes, 150 volumes, 192 volumes, 200 volumes, 250 volumes, 256 volumes, 300 volumes, 384 volumes, 400 volumes, 500 volumes, 512 volumes, 600 volumes, or more. For example, the method may be performed on a 96, 192, or 384 well plate. Furthermore, while this example provides contacting a sample with particles prior to partitioning, a method may alternatively comprise partitioning a sample (e.g., into separate wells of a well plate) prior to contacting with particles. Each sample volume may be separately mixed with particles prior to, concurrent with, or subsequent to addition into a partition. In particular cases, the particles are present in a partition (for example in dry form or in solution) prior to addition of the sample into the partition. In some cases, sample may be added to partitions comprising particles. For example, a well plate may be provided with particles, buffer, and reagents in dry form, such that a method of use may comprise adding solution to the wells to resuspend the particles and dissolve the buffer and reagents, and then adding sample to the wells.

An assay utilizing a plurality of particles may distinguish which particle a specific biomolecule, biomolecule fragment (e.g., peptide generated by digesting a biomolecule corona protein), or signal corresponding to a biomolecule (e.g., one of ten mass spectrometric signals associated with a specific peptide fragment of a biomolecule corona protein). As biomolecule corona composition is dependent on sample conditions (e.g., salinity, temperature, pH), biomolecular composition, and particle physicochemical properties, two particles may develop different biomolecule coronas upon contacting a sample. Accordingly, the type or types of particles on which a particular biomolecule is observed comprise biological state information which may be utilized for analysis. A method may identify the type of particle on which a biomolecule, biomolecule fragment, or signal corresponding to a biomolecule is observed. A method may identify a ratio of abundances of a biomolecule or biomolecule fragment on a plurality of particles. A method may identify a ratio of signal intensities associated with a biomolecule identified on a plurality of particles.

Annotating biomolecules, biomolecule fragments, and signals by particle type can increase the amount of information derived from an assay. While many methods generate lists of biomolecules associated with samples, the present disclosure provides methods which differentiate the binding affinity of individual biomolecules across multiple particle types.

A method (e.g., computer-implemented analysis with an (e.g., trained) classifier) of the present disclosure can comprise identifying a particle on which a biomolecule, biomolecule fragment, or signal was derived. A method of the present disclosure can comprise identifying an abundance ratio of a biomolecule or a biomolecule fragment across at least 2 particle types. A method of the present disclosure can comprise identifying an intensity ratio of a signal associated with a biomolecule or a biomolecule fragment across at least 2 particle types. A method of the present disclosure can comprise identifying an abundance ratio of a biomolecule or a biomolecule fragment across at least 3 particle types. A method of the present disclosure can comprise identifying an intensity ratio of a signal associated with a biomolecule or a biomolecule fragment across at least 3 particle types. A method of the present disclosure can comprise identifying an abundance ratio of a biomolecule or a biomolecule fragment across at least 4 particle types. A method of the present disclosure can comprise identifying an intensity ratio of a signal associated with a biomolecule or a biomolecule fragment across at least 4 particle types. A method of the present disclosure can comprise identifying an abundance ratio of a biomolecule or a biomolecule fragment across at least 5 particle types. A method of the present disclosure can comprise identifying an intensity ratio of a signal associated with a biomolecule or a biomolecule fragment across at least 5 particle types. A method of the present disclosure can comprise identifying an abundance ratio of a biomolecule or a biomolecule fragment across at least 6 particle types. A method of the present disclosure can comprise identifying an intensity ratio of a signal associated with a biomolecule or a biomolecule fragment across at least 6 particle types. A method of the present disclosure can comprise identifying an abundance ratio of a biomolecule or a biomolecule fragment across at least 8 particle types. A method of the present disclosure can comprise identifying an intensity ratio of a signal associated with a biomolecule or a biomolecule fragment across at least 8 particle types. A method of the present disclosure can comprise identifying an abundance ratio of a biomolecule or a biomolecule fragment across at least 10 particle types. A method of the present disclosure can comprise identifying an intensity ratio of a signal associated with a biomolecule or a biomolecule fragment across at least 10 particle types.

A method of the present disclosure may also identify an abundance or signal intensity ratio associated with different biomolecules or biomolecule fragments. For example, rather than exclusively utilizing an individual biomolecule abundance as an input, a trained classifier of the present disclosure may utilize an abundance ratio of a first biomolecule observed on a first particle and a second biomolecule observed on a second particle. As many biomolecules, and in particular many blood biomolecules, are ubiquitous across healthy and neurodegenerative disease samples (for example albumin, globulins, iron storage proteins), changes in their abundances may not be diagnostic for neurodegenerative disease states or progressions. However, a change in a ratio of two biomolecules, such as the iron storage proteins ferritin and transferrin can comprise information relevant for neurodegenerative disease and biological state diagnosis. Furthermore, as biomolecule particle adsorption can comprise a dependence on sample composition, an abundance or signal intensity ratio of two biomolecules on two particles can reflect biological state-relevant changes in a sample. Accordingly, a method of the present disclosure may identify an abundance ratio of a first biomolecule on a first particle and a second biomolecule on a second particle. A method of the present disclosure may also identify an intensity ratio of a first signal associated with a first biomolecule on a first particle and a second signal associated with a second biomolecule on a second particle.

Protein corona analysis may comprise an automated component. For example, an automated instrument may contact a sample with a particle or particle panel, identify proteins on the particle or particle panel (e.g., digest the proteins on the particle or particle panel and perform mass spectrometric analysis), and generate data for identifying a specific biomolecule or a biological state of a sample. The automated instrument may divide a sample into a plurality of volumes, and perform analysis on each volume or a subset of the plurality. The automated instrument may analyze multiple separate samples, for example by disposing multiple samples within multiple wells in a well plate, and performing parallel analysis on each sample or a subset of samples within the well plate.

The particle panels disclosed herein can be used to identify a number of proteins, peptides, protein groups, or protein classes using a protein analysis workflow described herein (e.g., a protein corona analysis workflow). Protein corona analysis may comprise contacting a sample to distinct particle types (e.g., a particle panel), forming biomolecule corona on the distinct particle types, and identifying the biomolecules in the biomolecule corona (e.g., by mass spectrometry). Feature intensities, as disclosed herein, refers to the intensity of a discrete spike (“feature”) seen on a plot of mass to charge ratio versus intensity from a mass spectrometry run of a sample. These features can correspond to variably ionized fragments of peptides and/or proteins. Using the data analysis methods described herein, feature intensities can be sorted into protein groups. Protein groups refer to two or more proteins that are identified by a shared peptide sequence. Alternatively, a protein group can refer to one protein that is identified using a unique identifying sequence. For example, if in a sample, a peptide sequence is assayed that is shared between two proteins (Protein 1: XYZZX and Protein 2: XYZYZ), a protein group could be the “XYZ protein group” having two members (protein 1 and protein 2) which share the identifiable XYZ motif. Alternatively, if the peptide sequence is unique to a single protein (Protein 1), a protein group could be the “ZZX” protein group having one member (Protein 1). A protein group can be supported by more than one peptide sequence. Protein detected or identified according to the instant disclosure can refer to a distinct protein detected in the sample (e.g., distinct relative other proteins detected using mass spectrometry). Thus, analysis of proteins present in distinct coronas corresponding to the distinct particle types in a particle panel yields a high number of feature intensities. In some cases, multiple features are associated with a single peptide, such that processing feature intensities yields a lower number of peptides. As an illustrative example, during data processing, 6000 feature intensities (e.g., mass spectrometric signals) may be assigned to 1200 peptides, yielding an average of one peptide per 5 feature intensities. Furthermore, in some cases, multiple peptides may be associated with individual proteins or protein groups, such that processing peptides yields a lower number of proteins or protein groups. As another illustrative example, 1200 peptides may be assigned to 300 protein groups, yielding an average of one protein group per 4 peptides. In some cases, a single feature intensity may identify a peptide. In some cases, a single peptide may identify a protein group. In some cases, a single feature intensity may be divided between multiple peptides. For example, tandem mass spectrometric analysis (MS/MS) of a feature intensity may identify that two separate peptides contribute to the feature intensity.

The methods disclosed herein include isolating one or more particle types from a sample or from more than one sample (e.g., a biological sample or a serially interrogated sample). The particle types can be isolated or separated from the sample using a magnet. Moreover, multiple samples that are spatially isolated can be processed in parallel. Thus, the methods disclosed herein provide for isolating or separating a particle type from unbound protein in a sample. A particle type may be separated using methods including but not limited to magnetic separation, centrifugation, filtration, or gravitational separation. Particle panels may be incubated with a plurality of spatially isolated samples, wherein each spatially isolated sample is in a well in a well plate (e.g., a 96-well plate, a 192-well plate, or a 384-well plate). After incubation, the particle types in each of the wells of the well plate can be separated from unbound protein present in the spatially isolated samples by placing the entire plate on a magnet. This pulls down the superparamagnetic particles in the particle panel. The supernatant in each sample can be removed to remove the unbound protein. These steps (incubate, pull down) can be repeated to effectively wash the particles, thus removing residual background unbound protein that may be present in a sample. This is one example, but one of skill in the art could envision numerous other scenarios in which superparamagnetic particles are rapidly isolated from one or more than one spatially isolated samples at the same time.

In some cases, the methods and compositions of the present disclosure may provide identification and measurement of particular proteins in the biological samples by processing of the proteomic data via digestion of coronas formed on the surface of particles. Examples of proteins that can be identified and measured include highly abundant proteins, proteins of medium abundance, and low-abundance proteins. A low abundance protein may be present in a sample at concentrations at or below about 10 ng/mL. A high abundance protein may be present in a sample at concentrations at or above about 10 μg/mL. A protein of moderate abundance may be present in a sample at concentrations between about 10 ng/ml and about 10 μg/mL. Examples of proteins that are highly abundant proteins include albumin, IgG, and the top 14 proteins in abundance that contribute 95% of the analyte mass in plasma. Additionally, any proteins that may be purified using a conventional depletion column may be directly detected in a sample using the particle panels disclosed herein. Examples of proteins may be any protein listed in published databases such as Keshishian et al. (Mol Cell Proteomics. 2015 September; 14(9): 2375-93. doi: 10.1074/mcp.M114.046813. Epub 2015 Feb. 27.), Farr et al. (J Proteome Res. 2014 Jan. 3; 13(1):60-75. doi: 10.1021/pr4010037. Epub 2013 Dec. 6.), or Pernemalm et al. (Expert Rev Proteomics. 2014 August; 11(4):431-48. doi: 10.1586/14789450.2014.901157. Epub 2014 Mar. 24.).

The proteomic data of the biological sample can be identified, measured, and quantified using a number of different analytical techniques. For example, proteomic data can be generated using SDS-PAGE or any gel-based separation technique. Peptides and proteins can also be identified, measured, and quantified using an immunoassay, such as ELISA. Alternatively, proteomic data can be identified, measured, and quantified using mass spectrometry, high performance liquid chromatography, LC-MS/MS, Edman Degradation, immunoaffinity techniques, methods disclosed in EP3548652, WO2019083856, WO2019133892, each of which is incorporated herein by reference in its entirety, and other protein separation techniques. In certain embodiments, proteomic data can be identified, measured, and quantified using LC-MS.

An assay may comprise protein collection of particles, protein digestion, and mass spectrometric analysis (e.g., MS, LC-MS, LC-MS/MS). The digestion may comprise chemical digestion, such as by cyanogen bromide or 2-Nitro-5-thiocyanatobenzoic acid (NTCB). The digestion may comprise enzymatic digestion, such as by trypsin or pepsin. The digestion may comprise enzymatic digestion by a plurality of proteases. The digestion may comprise a protease selected from among the group consisting of trypsin, chymotrypsin, Glu C, Lys C, elastase, subtilisin, proteinase K, thrombin, factor X, Arg C, papaine, Asp N, thermolysine, pepsin, aspartyl protease, cathepsin D, zinc mealloprotease, glycoprotein endopeptidase, proline, aminopeptidase, prenyl protease, caspase, kex2 endoprotease, or any combination thereof. The digestion may cleave peptides at random positions. The digestion may cleave peptides at a specific position (e.g., at methionines) or sequence (e.g., glutamate-histidine-glutamate). The digestion may enable similar proteins to be distinguished. For example, an assay may resolve 8 distinct proteins as a single protein group with a first digestion method, and as 8 separate proteins with distinct signals with a second digestion method. The digestion may generate an average peptide fragment length of 8 to 15 amino acids. The digestion may generate an average peptide fragment length of 12 to 18 amino acids. The digestion may generate an average peptide fragment length of 15 to 25 amino acids. The digestion may generate an average peptide fragment length of 20 to 30 amino acids. The digestion may generate an average peptide fragment length of 30 to 50 amino acids.

An assay may rapidly generate and analyze proteomic data. Beginning with an input biological sample (e.g., a buccal or nasal smear, plasma, or tissue), an assay of the present disclosure may generate and analyze proteomic data in less than 7 hours. Beginning with an input biological sample, an assay of the present disclosure may generate and analyze proteomic data in 5-7 hours. Beginning with an input biological sample, an assay of the present disclosure may generate and analyze proteomic data in less than 5 hours. Beginning with an input biological sample, an assay of the present disclosure may generate and analyze proteomic data in 3-5 hours. Beginning with an input biological sample, an assay of the present disclosure may generate and analyze proteomic data in 2-4 hours. Beginning with an input biological sample, an assay of the present disclosure may generate and analyze proteomic data in 2-3 hours. Beginning with an input biological sample, an assay of the present disclosure may generate and analyze proteomic data in less than 3 hours. Beginning with an input biological sample, an assay of the present disclosure may generate and analyze proteomic data in less than 2 hours. The analyzing may comprise identifying a protein group. The analyzing may comprise identifying a protein class. The analyzing may comprise quantifying an abundance of a biomolecule, a peptide, a protein, protein group, or a protein class. The analyzing may comprise identifying a ratio of abundances of two biomolecules, peptides, proteins, protein groups, or protein classes. The analyzing may comprise identifying a biological state.

### Dynamic Range

The biomolecule corona analysis methods described herein may comprise assaying biomolecules in a sample of the present disclosure across a wide dynamic range. The dynamic range of biomolecules assayed in a sample may be a range of measured signals of biomolecule abundances as measured by an assay method (e.g., mass spectrometry, chromatography, gel electrophoresis, spectroscopy, or immunoassays) for the biomolecules contained within a sample. For example, an assay capable of detecting proteins across a wide dynamic range may be capable of detecting proteins of very low abundance to proteins of very high abundance. The dynamic range of an assay may be directly related to the slope of assay signal intensity as a function of biomolecule abundance. For example, an assay with a low dynamic range may have a low (but positive) slope of the assay signal intensity as a function of biomolecule abundance, e.g., the ratio of the signal detected for a high abundance biomolecule to the ratio of the signal detected for a low abundance biomolecule may be lower for an assay with a low dynamic range than an assay with a high dynamic range. In specific cases, dynamic range may refer to the dynamic range of proteins within a sample or assaying method.

The particle panels disclosed herein can be used to identify the number of distinct proteins disclosed herein, and/or any of the specific proteins disclosed herein, over a wide dynamic range. As used herein, a dynamic range may denote a log₁₀ value of a ratio of the highest and lowest abundance species of a specified type. Enriching or assaying species over a dynamic range may refer to the abundances of those species in the sample from which they were assayed or derived. For example, the particle panels disclosed herein comprising distinct particle types, can enrich for proteins in a sample over the entire dynamic range at which proteins are present in a sample (e.g., a plasma sample). In some cases, a particle panel including any number of distinct particle types disclosed herein, enriches and identifies proteins over a dynamic range of about 2 to about 12. In some cases, a particle panel including any number of distinct particle types disclosed herein, enriches and identifies proteins over a dynamic range of about 3 to about 12. In some cases, a particle panel including any number of distinct particle types disclosed herein, enriches and identifies proteins over a dynamic range of about 4 to about 12. In some cases, a particle panel including any number of distinct particle types disclosed herein, enriches and identifies proteins over a dynamic range of a about 5 to about 12. In some cases, a particle panel including any number of distinct particle types disclosed herein, enriches and identifies proteins over a dynamic range of about 6 to about 12. In some cases, a particle panel including any number of distinct particle types disclosed herein, enriches and identifies proteins over a dynamic range of about 7 to about 12. In some cases, a particle panel including any number of distinct particle types disclosed herein, enriches and identifies proteins over a dynamic range of about 8 to about 12. In some cases, a particle panel including any number of distinct particle types disclosed herein, enriches and identifies proteins over a dynamic range of about 9 to about 12. In some cases, a particle panel including any number of distinct particle types disclosed herein, enriches and identifies proteins over a dynamic range of about 10 to about 12. In some cases, a particle panel including any number of distinct particle types disclosed herein, enriches and identifies proteins over a dynamic range of about 11 to about 12. In some cases, a particle panel including any number of distinct particle types disclosed herein, enriches and identifies proteins over a dynamic range of about 12. In some cases, a particle panel including any number of distinct particle types disclosed herein, enriches and identifies proteins over a dynamic range of from about 2 to about 6. In some cases, a particle panel including any number of distinct particle types disclosed herein, enriches and identifies proteins over a dynamic range of from about 3 to about 8. In some cases, a particle panel including any number of distinct particle types disclosed herein, enriches and identifies proteins over a dynamic range of from about 4 to 8. In some cases, a particle panel including any number of distinct particle types disclosed herein, enriches and identifies proteins over a dynamic range of from about 5 to about 10. In some cases, a particle panel including any number of distinct particle types disclosed herein, enriches and identifies proteins over a dynamic range of from about 6 to about 10. In some cases, a particle panel including any number of distinct particle types disclosed herein, enriches and identifies proteins over a dynamic range of from about 6 to about 12.

The biomolecule corona analysis methods described herein may compress the dynamic range of an assay. The dynamic range of an assay may be compressed relative to another assay if the slope of the assay signal intensity as a function of biomolecule abundance is lower than that of the other assay. For example, a plasma sample assayed using protein corona analysis with mass spectrometry may have a compressed dynamic range compared to a plasma sample assayed using mass spectrometry alone, directly on the sample or compared to provided abundance values for plasma proteins in databases (e.g., the database provided in Keshishian et al., Mol. Cell Proteomics 14, 2375-2393 (2015), also referred to herein as the “Carr database”). The compressed dynamic range may enable the detection of more low abundance biomolecules in a biological sample using biomolecule corona analysis with mass spectrometry than using mass spectrometry alone.

The dynamic range of a proteomic analysis assay may be the ratio of the signal produced by highest abundance proteins (e.g., the highest 10% of proteins by abundance) to the signal produced by the lowest abundance proteins (e.g., the lowest 10% of proteins by abundance). Compressing the dynamic range of a proteomic analysis may comprise decreasing the ratio of the signal produced by the highest abundance proteins to the signal produced by the lowest abundance proteins for a first proteomic analysis assay relative to that of a second proteomic analysis assay. The protein corona analysis assays disclosed herein may compress the dynamic range relative to the dynamic range of a total protein analysis method (e.g., mass spectrometry, gel electrophoresis, or liquid chromatography).

Provided herein are several methods for compressing the dynamic range of a biomolecular analysis assay to facilitate the detection of low abundance biomolecules relative to high abundance biomolecules. For example, a particle type of the present disclosure can be used to serially interrogate a sample. Upon incubation of the particle type in the sample, a biomolecule corona comprising forms on the surface of the particle type. If biomolecules are directly detected in the sample without the use of the particle types, for example by direct mass spectrometric analysis of the sample, the dynamic range may span a wider range of concentrations, or more orders of magnitude, than if the biomolecules are directed on the surface of the particle type. Thus, using the particle types disclosed herein may be used to compress the dynamic range of biomolecules in a sample. Without being limited by theory, this effect may be observed due to more capture of higher affinity, lower abundance biomolecules in the biomolecule corona of the particle type and less capture of lower affinity, higher abundance biomolecules in the biomolecule corona of the particle type.

A dynamic range of a proteomic analysis assay may be illustrated by the slope of a plot of a protein signal measured by the proteomic analysis assay as a function of total abundance of the protein in the sample. Compressing the dynamic range may comprise decreasing the slope of the plot of a protein signal measured by a proteomic analysis assay as a function of total abundance of the protein in the sample relative to the slope of the plot of a protein signal measured by a second proteomic analysis assay as a function of total abundance of the protein in the sample. The protein corona analysis assays disclosed herein may compress the dynamic range relative to the dynamic range of a total protein analysis method (e.g., mass spectrometry, gel electrophoresis, or liquid chromatography).

### Kits

Provided herein are kits comprising compositions of the present disclosure that may be used to perform the methods of the present disclosure. A kit may comprise one or more particle types to interrogate a sample to identify a biological state of a sample. In some cases, a kit may comprise a particle type provided in TABLES 1-5. A kit may comprise a reagent for functionalizing a particle (e.g., a reagent for tethering a small molecule functionalization to a particle surface). The kit may be pre-packaged in discrete aliquots. In some cases, the kit can comprise a plurality of different particle types that can be used to interrogate a sample. The plurality of particle types can be pre-packaged where each particle type of the plurality is packaged separately. Alternately, the plurality of particle types can be packaged together to contain combination of particle types in a single package. A particle may be provided in dried (e.g., lyophilized) form, or may be provided in a suspension or solution. The particles may be provided in a well plate. For example, a kit may contain an 8 well plate, an 8-384 well plate with particles provided (e.g., sealed) within the wells. For example, a well plate may comprise at least 8, at least 16, at least 24, at least 32, at least 40, at least 48, at least 56, at least 64, at least 72, at least 80, at least 88, at least 96, at least 104, at least 112, at least 120, at least 128, at least 136, at least 144, at least 152, at least 160, at least 168, at least 176, at least 184, at least 192, at least 200, at least 208, at least 216, at least 224, at least 232, at least 240, at least 248, at least 256, at least 264, at least 272, at least 280, at least 288, at least 296, at least 304, at least 312, at least 320, at least 328, at least 336, at least 344, at least 352, at least 360, at least 368, at least 376, at least 384, at least 392, at least 400 wells comprising particles. Two wells in such a well plate may contain different particles or different concentrations of particles. Two wells may comprise different buffers or chemical conditions. For example, a well plate may be provided with different particles in each row of wells and different buffers in each column of rows. A well may be sealed by a removable covering. For example, a kit may comprise a well plate comprising a plastic slip covering a plurality of wells. A well may be sealed by a pierceable covering. For example, a well may be covered by a septum that a needle can pierce to facilitate sample movement into and out of the well.

### Samples

The present disclosure provides a range of samples that can be assayed using the particles and the methods provided herein. A sample may be a biological sample (e.g., a sample derived from a living organism). A sample may comprise a cell or be cell-free. A sample may comprise a biofluid, such as blood, serum, plasma, urine, or cerebrospinal fluid (CSF). Samples of the present disclosure include biological samples from a subject. A method may include analyzing a sample from a single subject, or analyzing samples from multiple subjects. The subject may be a human or a non-human animal. The biological samples can contain a plurality of proteins or proteomic data, which may be analyzed after adsorption of proteins to the surface of the various sensor element (e.g., particle) types in a panel and subsequent digestion of protein coronas. Proteomic data can comprise nucleic acids, peptides, or proteins. A biofluid may be a fluidized solid, for example a tissue homogenate, or a fluid extracted from a biological sample. A biological sample may be, for example, a tissue sample or a fine needle aspiration (FNA) sample. A biological sample may be a cell culture sample. For example, a biofluid may be a fluidized cell culture extract.

A wide range of samples are compatible for use within the methods and compositions of the present disclosure. The biological sample may comprise plasma, serum, urine, cerebrospinal fluid, synovial fluid, tears, saliva, whole blood, a blood component (e.g., plasma or white blood cells), milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. The biological sample may comprise blood or a blood component. The biological sample may comprise multiple biological samples (e.g., pooled plasma from multiple subjects, or multiple tissue samples from a single subject). The biological sample may comprise a single type of biofluid or biomaterial from a single source. A biological sample may comprise a nerve biopsy.

Various methods of the present disclosure utilize blood or blood components (e.g., red blood cells, buffy coats, plasma). Contrasting many tissue biopsies, which can be damaging and cost intensive, blood collection is often relatively facile and benign, and is therefore suitable for routine and low-risk patient monitoring. Furthermore, as human blood is estimated to contain over 5000 types of protein groups whose abundances and forms (e.g., post-translationally modifications and variant types) can be responsive to, the blood proteome offers a biological state changes are often evidenced by subtle changes in blood protein composition. A method of the present disclosure may use whole blood (e.g., untreated blood drawn from a subject). A method of the present disclosure may also use a treated or partitioned blood sample. In some cases, a sample comprises plasma, buffy coat, white blood cells, platelets, hematocrit, red blood cells, serum, blood clots or any combination thereof. In some cases, plasma, buffy coat, white blood cells, platelets, hematocrit, red blood cells, serum, blood clots or any combination thereof are extracted from a blood sample for use in a method disclosed herein.

In some cases, a method utilizes serum. As used herein, “serum” may denote the liquid fraction remaining after a blood sample clots. As a blood sample left at room temperature will typically clot within 15-60 minutes, serum may be prepared by incubating a blood sample at or above room temperature, for example at 25° C. or at 37° C., respectively. After at least about 10 minutes, at least about 15 minutes, at least about 20 minutes, at least about 30 minutes, at least about 40 minutes, at least about 50 minutes, or at least about 60 minutes, the blood clots may be separated from solution through centrifugation. While serum is often prepared non-hemolyzed (e.g., wherein blood cells remain intact through clotting and removal), some methods of the present disclosure may utilize serum derived from hemolyzed blood samples.

In some cases, a method utilizes plasma. As used herein, “plasma” may denote a fraction collected from blood pretreated with an anticoagulant and separated from blood cells and platelets. Contrasting with serum, plasma typically contains an array of clotting factors, such as fibrinogen, prothrombin, and proaccelerin. As the concentrations and forms of these species can reflect certain health conditions, plasma analysis can provide greater diagnostic insight than serum analysis for some biological states. Plasma samples can be prepared treating blood with an anticoagulant, and then centrifuging the treated blood. The anticoagulant may comprise citrate, ethylenediaminetetraaceticacid (EDTA), potassium oxalate, hirudin, argatroban, ximelagatran, heparin, fondaparinux, or any combination thereof.

Centrifugation parameters affect the proteins which remain in solution, and therefore may be modified depending on the biomolecules of interest for detection from plasma or serum. Centrifugation may be performed for at least 2 minutes, at least 4 minutes, at least 6 minutes, at least 8 minutes, at least 10 minutes, at least 12 minutes, at least 15 minutes, at least 20 minutes, or at least 30 minutes. Centrifugation may be performed for at most 30 minutes, at most 20 minutes, at most 15 minutes, at most 10 minutes, at most 8 minutes, at most 6 minutes, at most 4 minutes, or at most 2 minutes. Centrifugation may impart at least 100 gravitational force equivalents (g), at least 200 g, at least 300 g, at least 400 g, at least 500 g, at least 600 g, at least 800 g, at least 1000 g, at least 1200 g, at least 1500 g, at least 1800 g, at least 2000 g, at least 2500 g, at least 3000 g, at least 4000 g, at least 5000 g, at least 6000 g, at least 8000 g, or at least 10000 g. The centrifugation may impart at most 100 g, at most 200 g, at most 300 g, at most 400 g, at most 500 g, at most 600 g, at most 800 g, at most 1000 g, at most 1200 g, at most 1500 g, at most 1800 g, at most 2000 g, at most 2500 g, at most 3000 g, at most 4000 g, at most 5000 g, at most 6000 g, at most 8000 g, or at most 10000 g.

The biological sample may be diluted or pre-treated. The biological sample may undergo depletion (e.g., albumin removal from serum or plasma) prior to or following contact with a particle or plurality of particles. The biological sample may also undergo physical (e.g., homogenization or sonication) or chemical treatment prior to or following contact with a particle or plurality of particles. The biological sample may be diluted prior to or following contact with a particle or plurality of particles. The dilution medium may comprise buffer or salts, or be purified water (e.g., distilled water). Different partitions of a biological sample may undergo different degrees of dilution. A biological sample or a portion thereof may undergo a 1.1-fold, 1.2-fold, 1.3-fold, 1.4-fold, 1.5-fold, 2-fold, 3-fold, 4-fold, 5-fold, 6-fold, 8-fold, 10-fold, 12-fold, 15-fold, 20-fold, 30-fold, 40-fold, 50-fold, 75-fold, 100-fold, 200-fold, 500-fold, or 1000-fold dilution. For example, a plasma sample may be subjected to a 5-fold dilution with buffer prior to analysis.

The compositions and methods of the present disclosure can be used to measure, detect, and identify specific proteins from biological samples. Examples of proteins that can be identified and measured include highly abundant proteins, proteins of medium abundance, and low-abundance proteins. For example, a composition or method may identify at least 1, at least 2, at least 3, at least 4, at least 5, at least 6, at least 7, at least 8, at least 10, at least 12, at least 15, at least 18, at least 20, at least 25, at least 30, at least 35, at least 40, or at least 50 human plasma proteins from the group consisting of those described in TABLE 6, TABLE 7, or TABLE 8.

### Neurodegenerative Disease Detection

The compositions and methods disclosed herein can be used to identify various biological states of samples and subjects from which samples are derived. As an example, biological state can refer to an elevated or low level of a particular biomolecule or set of biomolecules, such as elevated blood glucose or misfolded alpha synuclein. Biological state may also refer to a particular pathology, such as Alzheimer's disease, or a stage of the pathology, such as early, middle, or late stage dementia. In other examples, a biological state can refer to identification of a disease, such as cancer. The particles and methods of us thereof can be used to distinguish between two biological states. The two biological states may be related diseases states (e.g., mild cognitive impairment and Alzheimer's disease). The two biological states may be different phases of a disease, such as pre-Alzheimer's and mild Alzheimer's. The two biological states may be distinguished with a high degree of accuracy (e.g., the percentage of accurately identified biological states among a population of samples). For example, the compositions and methods of the present disclosure may distinguish two biological states with at least 60% accuracy, at least 70% accuracy, at least 75% accuracy at least 80% accuracy, at least 85% accuracy, at least 90% accuracy, at least 95% accuracy, at least 98% accuracy, or at least 99% accuracy. The two biological states may be distinguished with a high degree of specificity (e.g., the rate at which negative results are correctly identified among a population of samples). For example, the compositions and methods of the present disclosure may distinguish two biological states with at least 60% specificity, at least 70% specificity, at least 75% specificity at least 80% specificity, at least 85% specificity, at least 90% specificity, at least 95% specificity, at least 98% specificity, or at least 99% specificity.

The methods, compositions, and systems of the present disclosure may detect a neurological disease state. Neurological disorders or neurological diseases are used interchangeably and refer to diseases associated with neurological tissues, such as the brain, the spinal cord, and the nerves that connect them. Neurological diseases include, but are not limited to, brain tumors, epilepsy, Parkinson's disease, Alzheimer's disease, ALS, arteriovenous malformation, cerebrovascular disease, brain aneurysms, epilepsy, multiple sclerosis, Peripheral Neuropathy, Post-Herpetic Neuralgia, stroke, dementia (e.g., frontotemporal dementia), demyelinating disease (including but are not limited to, multiple sclerosis, Devic's disease (i.e. neuromyelitis optica), central pontine myelinolysis, progressive multifocal leukoencephalopathy, leukodystrophies, Guillain-Barre syndrome, progressing inflammatory neuropathy, Charcot-Marie-Tooth disease, chronic inflammatory demyelinating polyneuropathy, and anti-MAG peripheral neuropathy) and the like. Neurological disorders also include immune-mediated neurological disorders (IMNDs), which include diseases with at least one component of the immune system reacts against host proteins present in the central or peripheral nervous system and contributes to disease pathology. IMNDs may include, but are not limited to, demyelinating disease, paraneoplastic neurological syndromes, immune-mediated encephalomyelitis, immune-mediated autonomic neuropathy, myasthenia gravis, autoantibody-associated encephalopathy, and acute disseminated encephalomyelitis. In some embodiments, the neurological disease is Alzheimer's disease. In some embodiments, the methods, compositions, and systems of the present disclosure may detect a neurodegenerative disease. In some embodiments, the neurodegenerative disease is dementia. In some embodiments, the neurodegenerative disease is Alzheimer's disease related dementia (ADRD).

Methods, systems, and/or apparatuses of the present disclosure may be able to accurately distinguish between patients with or without Alzheimer's disease (e.g., identify between a healthy biological state and Alzheimer's disease). These may also be able to detect patients who are pre-symptomatic and may develop Alzheimer's disease several years after the screening. This provides advantages of being able to treat a disease at a very early stage, even before development of the disease.

The methods, compositions, and systems of the present disclosure can detect a pre-disease stage of a disease or disorder. A pre-disease stage is a stage at which the patient has not developed any signs or symptoms of the disease. A pre-neurological disease stage would be a stage in which a person has not developed one or more symptom of the neurological disease. The ability to diagnose a disease before one or more sign or symptom of the disease is present allows for close monitoring of the subject and the ability to treat the disease at a very early stage, increasing the prospect of being able to halt progression or reduce the severity of the disease.

The methods, compositions, and systems of the present disclosure may detect the early stages of a disease or disorder. Early stages of the disease can refer to when the first signs or symptoms of a disease may manifest within a subject. The early stage of a disease may be a stage at which there are no outward signs or symptoms. For example, in Alzheimer's disease an early stage may be a pre-Alzheimer's stage in which no symptoms are detected yet the patient will develop Alzheimer's months or years later.

Identifying a disease in either pre-disease development or in the early states may often lead to a higher likelihood for a positive outcome for the patient. For example, diagnosing dementia at an early stage (stage 0 or stage 1) can enable early stage interventions, which may slow or even halt its progression, and increase the quality of life and life expectancy of the patient.

In some cases, the methods, compositions, and systems of the present disclosure are able to detect intermediate stages of the disease. Intermediate states of the disease describe stages of the disease that have passed the first signs and symptoms and the patient is experiencing one or more symptom of the disease. Further, the methods, compositions, and systems of the present disclosure may be able to detect late or advanced stages of the disease. Late or advanced stages of the disease may also be called “severe” or “advanced” and usually indicates that the subject is suffering from multiple symptoms and effects of the disease.

The methods of the present disclosure can include processing the biomolecule corona data of a sample against a collection of biomolecule corona datasets representative of a plurality of diseases and/or a plurality of disease states to determine if the sample indicates a disease and/or disease state. For example, samples can be collected from a population of subjects over time. Once the subjects develop a disease or disorder, the present disclosure allows for the ability to characterize and detect the changes in biomolecule fingerprints over time in the subject by computationally analyzing the biomolecule fingerprint of the sample from the same subject before they have developed a disease to the biomolecule fingerprint of the subject after they have developed the disease. Samples can also be taken from cohorts of patients who all develop the same disease, allowing for analysis and characterization of the biomolecule fingerprints that are associated with the different stages of the disease for these patients (e.g. from pre-disease to disease states).

In some cases, the methods, compositions, and systems of the present disclosure are able to distinguish not only between different types of diseases, but also between the different stages of the disease (e.g. early stages of disease). This can comprise distinguishing healthy subjects from pre-disease state subjects. The pre-disease state may be, for example, a neurodegenerative disease, dementia.

In some embodiments, the methods, compositions, and systems of the present disclosure are able to identify progression of a neurodegenerative disease, such as dementia. In some instances, the methods comprise tracking the rate of dementia progression. In some embodiments, the biofluid sample has an increased rate of dementia progression (e.g., associated with a shorter time to CDRg increase). In some embodiments, the biofluid sample has a decreased rate of dementia progression (e.g., associated with a delayed time to CDRg increase).

### Computer Control Systems

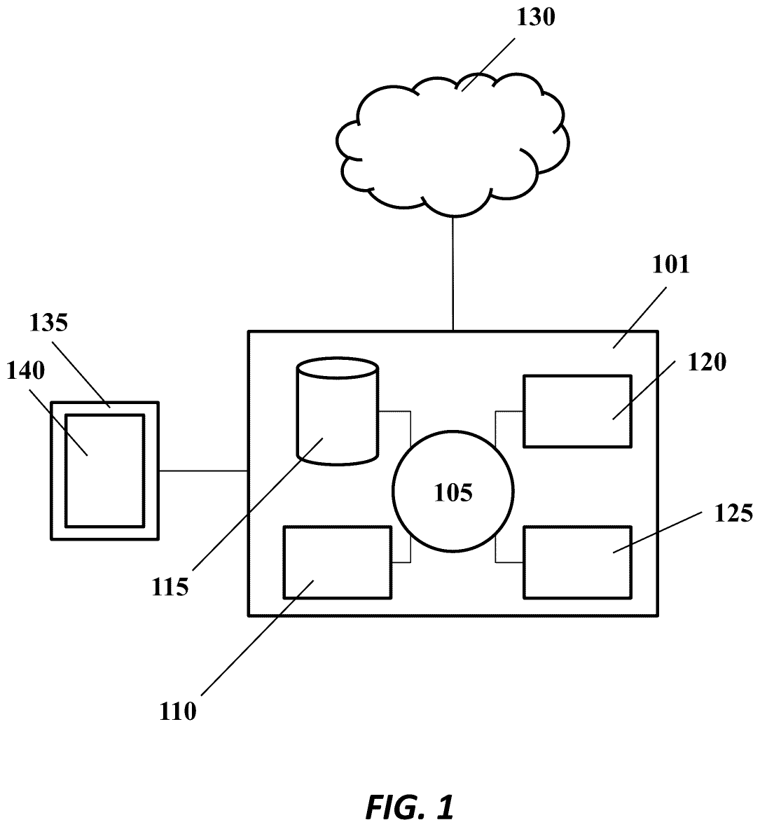

The present disclosure provides computer control systems that are programmed to implement methods of the disclosure. FIG. 1 shows a computer system that is programmed or otherwise configured to implement methods provided herein. The computer system 101 can regulate various aspects of the assays disclosed herein, which are capable of being automated (e.g., movement of any of the reagents disclosed herein on a substrate). The computer system 101 can be an electronic device of a user or a computer system that is remotely located with respect to the electronic device. The electronic device can be a mobile electronic device.

The computer system 101 includes a central processing unit (CPU, also “processor” and “computer processor” herein) 105, which can be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 can be a data storage unit (or data repository) for storing data. The computer system 101 can be operatively coupled to a computer network (“network”) 130 with the aid of the communication interface 120. The network 130 can be the Internet, an internet and/or extranet, or an intranet and/or extranet that is in communication with the Internet. The network 130 in some cases is a telecommunication and/or data network. The network 130 can include one or more computer servers, which can enable distributed computing, such as cloud computing. The network 130, in some cases with the aid of the computer system 101, can implement a peer-to-peer network, which may enable devices coupled to the computer system 101 to behave as a client or a server.

The CPU 105 can execute a sequence of machine-readable instructions, which can be embodied in a program or software. The instructions may be stored in a memory location, such as the memory 110. The instructions can be directed to the CPU 105, which can subsequently program or otherwise configure the CPU 105 to implement methods of the present disclosure. Examples of operations performed by the CPU 105 can include fetch, decode, execute, and writeback.

The CPU 105 can be part of a circuit, such as an integrated circuit. One or more other components of the system 101 can be included in the circuit. In some cases, the circuit is an application specific integrated circuit (ASIC).

The storage unit 115 can store files, such as drivers, libraries and saved programs. The storage unit 115 can store user data, e.g., user preferences and user programs. The computer system 101 in some cases can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 101 via the network 130.

Methods as described herein can be implemented by way of machine (e.g., computer processor) executable code stored on an electronic storage location of the computer system 101, such as, for example, on the memory 110 or electronic storage unit 115. The machine executable or machine readable code can be provided in the form of software. During use, the code can be executed by the processor 105. In some cases, the code can be retrieved from the storage unit 115 and stored on the memory 110 for ready access by the processor 105. In some situations, the electronic storage unit 115 can be precluded, and machine-executable instructions are stored on memory 110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 135 that comprises a user interface (UI) 140 for providing, for example a readout of the proteins identified using the methods disclosed herein. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 105.

Classifiers as described herein may be used for determination, analysis, or statistical classification in the methods provided herein. For example, may include, but are not limited to, for example, a supervised and unsupervised data analysis and clustering approaches such as hierarchical cluster analysis (HCA), principal component analysis (PCA), Partial least squares Discriminant Analysis (PLSDA), machine learning (e.g., Random Forest), logistic regression, decision trees, support vector machine (SVM), k-nearest neighbors, naive Bayes, linear regression, polynomial regression, SVM for regression, K-means clustering, and hidden Markov models, among others. In some embodiments, the classifier includes logistic regression, such as described in Example 1. In some embodiments, the classifier includes Cox models, such as described in Example 1. In some embodiments, Cox (e.g., regression) models include Cox proportion hazards (CPH) models. In some embodiments, Cox models include Cox time-varying (CTV) regression models. The computer system can perform various aspects of analyzing the protein sets or protein corona of the present disclosure, such as, for example, comparing/analyzing the biomolecule corona of several samples to determine with statistical significance what patterns are common between the individual biomolecule coronas to determine a protein set that is associated with the biological state. The computer system can be used to develop classifiers to detect and discriminate different protein sets or protein corona (e.g., characteristic of the composition of a protein corona). Data collected from the presently disclosed sensor array can be used to train a machine learning algorithm, specifically an algorithm that receives array measurements from a patient and outputs specific biomolecule corona compositions from each patient. Before training the algorithm, raw data from the array can be first denoised to reduce variability in individual variables.

Machine learning can be generalized as the ability of a learning machine to perform accurately on new, unseen examples/tasks after having experienced a learning data set. Machine learning may include the following concepts and methods. Supervised learning concepts may include AODE; Artificial neural network, such as Backpropagation, Autoencoders, Hopfield networks, Boltzmann machines, Restricted Boltzmann Machines, and Spiking neural networks; Bayesian statistics, such as Bayesian network and Bayesian knowledge base; Case-based reasoning; Gaussian process regression; Gene expression programming; Group method of data handling (GMDH); Inductive logic programming; Instance-based learning; Lazy learning; Learning Automata; Learning Vector Quantization; Logistic Model Tree; Minimum message length (decision trees, decision graphs, etc.), such as Nearest Neighbor Algorithm and Analogical modeling; Probably approximately correct learning (PAC) learning; Ripple down rules, a knowledge acquisition methodology; Symbolic machine learning algorithms; Support vector machines; Random Forests; Ensembles of classifiers, such as Bootstrap aggregating (bagging) and Boosting (meta-algorithm); Ordinal classification; Information fuzzy networks (IFN); Conditional Random Field; ANOVA; Linear classifiers, such as Fisher's linear discriminant, Linear regression, Logistic regression, Multinomial logistic regression, Naive Bayes classifier, Perceptron, Support vector machines; Quadratic classifiers; k-nearest neighbor; Boosting; Decision trees, such as C4.5, Random forests, ID3, CART, SLIQ SPRINT; Bayesian networks, such as Naive Bayes; and Hidden Markov models. Unsupervised learning concepts may include; Expectation-maximization algorithm; Vector Quantization; Generative topographic map; Information bottleneck method; Artificial neural network, such as Self-organizing map; Association rule learning, such as, Apriori algorithm, Eclat algorithm, and FPgrowth algorithm; Hierarchical clustering, such as Singlelinkage clustering and Conceptual clustering; Cluster analysis, such as, K-means algorithm, Fuzzy clustering, DBSCAN, and OPTICS algorithm; and Outlier Detection, such as Local Outlier Factor. Semi-supervised learning concepts may include; Generative models; Low-density separation; Graph-based methods; and Co-training. Reinforcement learning concepts may include; Temporal difference learning; Q-learning; Learning Automata; and SARSA. Deep learning concepts may include; Deep belief networks; Deep Boltzmann machines; Deep Convolutional neural networks; Deep Recurrent neural networks; and Hierarchical temporal memory. In some instances, the machine learning comprises logistic regression-based machine learning, such as described in Example 1. A computer system may be adapted to implement a method described herein. The system includes a central computer server that is programmed to implement the methods described herein. The server includes a central processing unit (CPU, also “processor”) which can be a single core processor, a multi core processor, or plurality of processors for parallel processing. The server also includes memory (e.g., random access memory, read-only memory, flash memory); electronic storage unit (e.g. hard disk); communications interface (e.g., network adaptor) for communicating with one or more other systems; and peripheral devices which may include cache, other memory, data storage, and/or electronic display adaptors. The memory, storage unit, interface, and peripheral devices are in communication with the processor through a communications bus (solid lines), such as a motherboard. The storage unit can be a data storage unit for storing data. The server is operatively coupled to a computer network (“network”) with the aid of the communications interface. The network can be the Internet, an intranet and/or an extranet, an intranet and/or extranet that is in communication with the Internet, a telecommunication or data network. The network in some cases, with the aid of the server, can implement a peer-to-peer network, which may enable devices coupled to the server to behave as a client or a server.

The storage unit can store files, such as subject reports, and/or communications with the data about individuals, or any aspect of data associated with the present disclosure.

The computer server can communicate with one or more remote computer systems through the network. The one or more remote computer systems may be, for example, personal computers, laptops, tablets, telephones, Smart phones, or personal digital assistants.

In some applications the computer system includes a single server. In other situations, the system includes multiple servers in communication with one another through an intranet, extranet and/or the internet.

The server can be adapted to store measurement data or a database as provided herein, patient information from the subject, such as, for example, medical history, family history, demographic data and/or other clinical or personal information of potential relevance to a particular application. Such information can be stored on the storage unit or the server and such data can be transmitted through a network.

Methods as described herein can be implemented by way of machine (or computer processor) executable code (or software) stored on an electronic storage location of the server, such as, for example, on the memory, or electronic storage unit. During use, the code can be executed by the processor. In some cases, the code can be retrieved from the storage unit and stored on the memory for ready access by the processor. In some situations, the electronic storage unit can be precluded, and machine-executable instructions are stored on memory. Alternatively, the code can be executed on a second computer system.

Aspects of the systems and methods provided herein, such as the server, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless likes, optical links, or the like, also may be considered as media bearing the software. As used herein, unless restricted to non-transitory, tangible “storage” media, terms such as computer or machine “readable medium” can refer to any medium that participates in providing instructions to a processor for execution.

The computer systems described herein may comprise computer-executable code for performing any of the algorithms or algorithms-based methods described herein. In some applications the algorithms described herein will make use of a memory unit that is comprised of at least one database.

Data relating to the present disclosure can be transmitted over a network or connections for reception and/or review by a receiver. The receiver can be but is not limited to the subject to whom the report pertains; or to a caregiver thereof, e.g., a health care provider, manager, other health care professional, or other caretaker; a person or entity that performed and/or ordered the analysis. The receiver can also be a local or remote system for storing such reports (e.g. servers or other systems of a “cloud computing” architecture). In one embodiment, a computer-readable medium includes a medium suitable for transmission of a result of an analysis of a biological sample using the methods described herein.

Aspects of the systems and methods provided herein can be embodied in programming. Various aspects of the technology may be thought of as “products” or “articles of manufacture” typically in the form of machine (or processor) executable code and/or associated data that is carried on or embodied in a type of machine readable medium. Machine executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide nontransitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

### Computer-Implemented Systems

Further disclosed herein are computer-implemented systems for identifying biological state information from biomolecule corona data. The computer-implemented system may comprise a communication interface configured to receive data, such as biomolecule corona data. The communication interface may receive data over a communication network, such as a cloud-based network or a computer server-based network, or a storage device such as a flash drive memory device or a compact disc. The computer-implemented system may comprise a computer in communication with the communication interface. The computer may comprise one or more processors, as well as computer readable medium comprising machine-executable code which may be executed by the one or more processors, and which may be configured to implement a method. The method may process biomolecule corona data, for example by filtering or baseline correcting a portion of the data. The method may identify a biomolecule (e.g., a protein, a protein group, a saccharide, a nucleic acid, or a metabolite). The method may identify an abundance of a biomolecule or an intensity of a signal (e.g., by performing a Gaussian or Lorentzian fit to a peak in the data). The method may identify a ratio of two or more biomolecule abundances or two or more signal intensities. The method may comprise a machine learning algorithm or a trained algorithm for biological state analysis. The method may identify a biological state (e.g., healthy or Alzheimer's) based at least in part on the biomolecule corona data. In some embodiments, the method may identify the progression (or risk thereof) of a disease described elsewhere herein based at least in part on the biomolecule corona data.

The computer may comprise one or more processors, as well as computer readable medium which may be executed by the one or more processors to communicate with an instrument through the communication interface, and operate or provide parameters (e.g., temperatures, incubation times, number of wash cycles) the instrument to perform biomolecule corona analysis (e.g., perform biological sample-particle incubation, wash, digestion, and solid-phase extraction). For example, upon input of a sample and reagents into an automated instrument for biomolecule corona analysis, the computer may prompt a user for information regarding the sample or intended assay, and then execute a biomolecule corona analysis method based on the information by the user, such as sample type, intended depth of sample coverage (e.g., in some cases, the length of particle-biological sample incubation times may affect the number of protein groups identified in an assay).

The computer may comprise one or more processors, as well as computer readable medium which may be executed by the one or more processors to communicate with an instrument configured to analyze a sample which has been subjected to biomolecule corona analysis through the communication interface, and to operate or provide parameters to the instrument, as well as computer readable medium which may be executed by the one or more processors to operate an instrument configured to perform biomolecule corona analysis. For example, the computer may provide parameters to a mass spectrometer for analysis of a protease digested biomolecule corona.

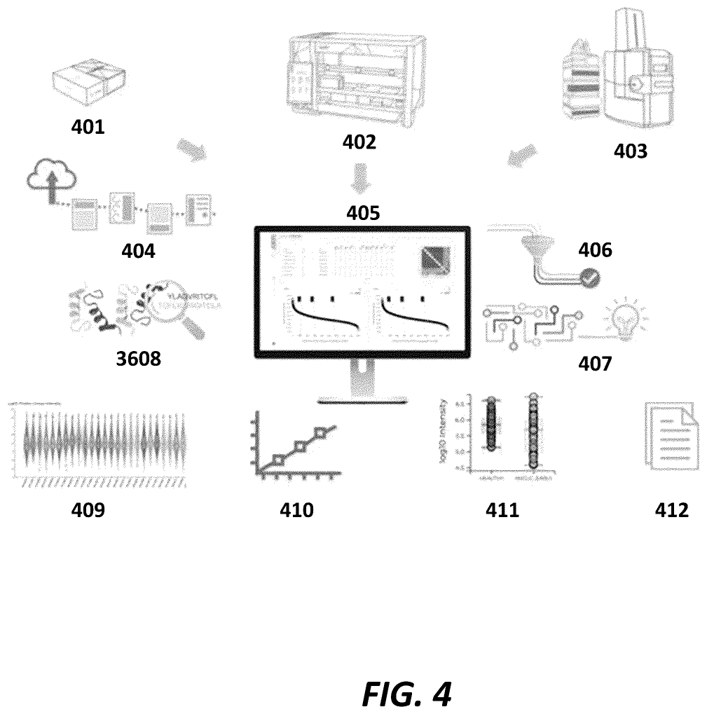

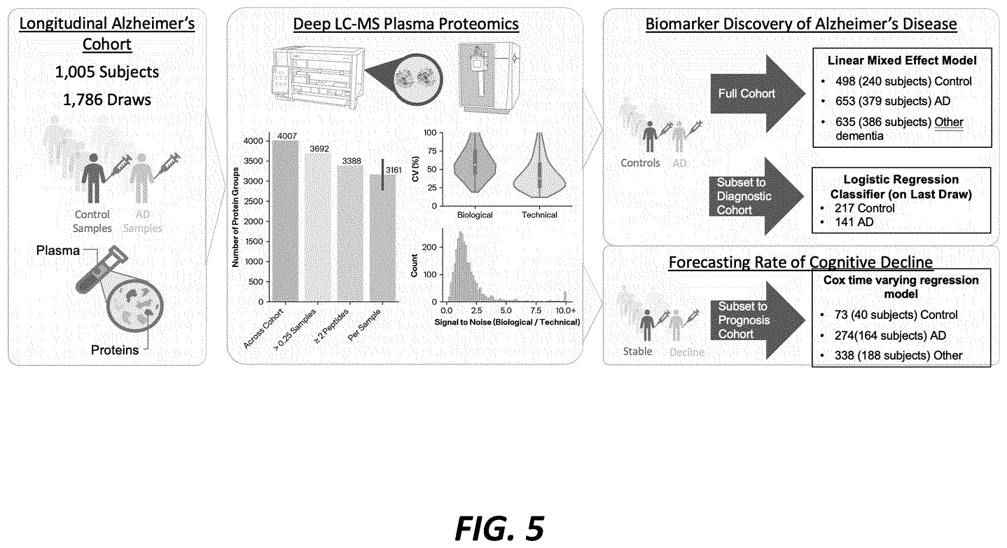

FIG. 4 illustrates a workflow utilizing assay instrumentation and materials and a computer-implemented system of the present disclosure. An assay kit 401 comprising reagents for biomolecule corona analysis, an instrument configured to perform automated biomolecule corona analysis 402, and an analytical instrument 403 for identifying biomolecules from a biomolecule corona analysis method (e.g., a mass spectrometer) may be used to generate biomolecule corona data. A computer 404 may communicate with the instrument configured to perform automated biomolecule corona analysis 402, the analytical instrument 403, or both instruments. The computer 404 may provide parameters to or operate one or both instruments. The computer may receive data from the instrument configured to perform automated biomolecule corona analysis 402, the analytical instrument 403, or both. The computer 404 may be in communication with a server 405 (e.g., a cloud-based server), and may be configured to upload data to the server 405. The computer 404 may comprise one or more processors, as well as computer readable medium comprising machine-executable code which may be executed by the one or more processors, and which may be configured to implement a method 406 for analyzing data from the instrument configured to perform automated biomolecule corona analysis 402, the analytical instrument 403, or both. The machine-executable code may also be configured to identify and annotate 407 biomolecules from the biomolecule corona data. The computer may be configured to display 408 the analyzed data or unanalyzed data, display metrics generated from analysis of the data 409, display performance metrics 410 (e.g., performance metrics derived from analysis of the data or received from the instrument configured to perform automated biomolecule corona analysis 402, the analytical instrument 403, or both. The machine-executable code may be configured to identify abundance ratios 411 of species identified or annotated 407. The machine-executable code may generate results files 412, which may be transmitted through to the server 405, to another device in communication with the computer 404, such as a flash drive memory device.

### Classifiers for Neurodegenerative Disease Analysis

The method of determining a set of proteins associated with the disease or disorder and/or disease state include the analysis of the corona of the at least one sample. This determination, analysis or statistical classification can be performed using methods, including, but not limited to, for example, supervised and unsupervised data analysis, machine learning, deep learning, and clustering approaches including hierarchical cluster analysis (HCA), principal component analysis (PCA), Partial least squares Discriminant Analysis (PLS-DA), random forest, logistic regression, Cox regression, decision trees, support vector machine (SVM), k-nearest neighbors, naive bayes, linear regression, polynomial regression, SVM for regression, K-means clustering, and hidden Markov models, among others. In other words, the proteins in the corona of the sample can be compared/analyzed with another sample, a control sample, or another set of data, to determine with statistical significance what patterns are common between the individual corona to determine a set of proteins that is associated with the disease or disorder or disease state. In some instances, the proteins in the corona of the sample can be compared/analyzed with another sample, a control sample, or another set of data, to determine with statistical significance what patterns are common between the individual corona to determine a set of proteins that are associated with disease progression (e.g., progression of dementia).

Generally, machine learning algorithms are used to construct models that accurately assign class labels to datasets or features within datasets based on a set of input features. In some case it may be advantageous to employ machine learning and/or deep learning approaches for the methods described herein. For example, machine learning can be used to associate the protein corona with various disease states (e.g. no disease, precursor to a disease, having early or late stage of the disease, etc.). For example, in some cases, one or more machine learning algorithms are employed in connection with a method of the invention to analyze data detected and obtained by the protein corona and sets of proteins derived therefrom. For example, a machine learning algorithm may be trained to distinguish subjects with Alzheimer's disease from healthy subjects.

A method or system (e.g., a computer-implemented system) may utilize biomolecule corona data for classifier training and as an input on which a trained classifier may perform analysis. The biomolecule corona data may comprise raw data (data acquired directly from an instrument such as a mass spectrometer, or data which has been subjected to basic pre-processing and filtering steps, such as baseline flattening), processed data (e.g., a list of mass spectrometry peaks identified above a baseline signal-to-noise threshold, a ratio of two mass spectrometry peak intensities), annotated data (e.g., a list of peptides identified from mass spectrometric data), or any combination thereof. As the present disclosure provides methods for identifying biomolecules spanning broad dynamic ranges, biomolecule corona data used for training or biological sample analysis may span about 2 to about 12 orders of magnitude in terms of biomolecule concentration in the biological sample, about 4 to about 12 orders of magnitude in terms of biomolecule concentration in the biological sample, about 5 to about 12 orders of magnitude in terms of biomolecule concentration in the biological sample, about 6 to about 12 orders of magnitude in terms of biomolecule concentration in the biological sample, about 7 to about 12 orders of magnitude in terms of biomolecule concentration in the biological sample, about 8 to about 12 orders of magnitude in terms of biomolecule concentration in the biological sample, about 4 to about 10 orders of magnitude in terms of biomolecule concentration in the biological sample, about 5 to about 10 orders of magnitude in terms of biomolecule concentration in the biological sample, about 6 to about 10 orders of magnitude in terms of biomolecule concentration in the biological sample, about 7 to about 10 orders of magnitude in terms of biomolecule concentration in the biological sample, about 8 to about 10 orders of magnitude in terms of biomolecule concentration in the biological sample, about 2 to about 8 orders of magnitude in terms of biomolecule concentration in the biological sample, about 4 to about 8 orders of magnitude in terms of biomolecule concentration in the biological sample, about 6 to about 8 orders of magnitude in terms of biomolecule concentration in the biological sample, about 2 to about 6 orders of magnitude in terms of biomolecule concentration in the biological sample, about 4 to about 6 orders of magnitude in terms of biomolecule concentration in the biological sample, about 2 to about 4 orders of magnitude in terms of biomolecule concentration in the biological sample, or about 2 to about 3 orders of magnitude in terms of biomolecule concentration in the biological sample.

Aspects of the present disclosure increase the amount of information derived from biological sample analysis. Some biological states are not distinguishable solely through biomolecule identification. For example, identifying concentrations for the thirty most abundant proteins in a plasma sample is often insufficient for distinguishing subjects afflicted with Alzheimer's disease from healthy subjects. The present disclosure provides a range of approaches for increasing the dimensionality of biological sample data, and for using the data to identify biological states. In some cases, biomolecule corona data may comprise a ratio of two or more biomolecule abundances or signal intensities. For example, a datapoint may be a ratio of three mass spectrometric peak intensities, and which may comprise greater diagnostic utility than the intensities of all three mass spectrometric peak intensities taken individually.

In some cases, biomolecule corona data comprises particle-level annotations which identify the type of particle a biomolecule was identified on, and further may optionally comprise an abundance of or a signal intensity associated with the biomolecule. For example, in some cases, alpha-2-antiplasmin plasma levels may be weakly diagnostic for Alzheimer's disease, but alpha-2-antiplasmin abundance in biomolecule coronas of a (PDMAPMA)-coated SPION contacted to plasma may vary with a high degree of statistical significance between healthy and Alzheimer's disease samples. In some cases, biomolecule corona data comprises particle-level annotations which identify the type of particle a peptide was identified on. In some cases, a plurality of peptides from a single protein are identified on a single particle. In some cases, biomolecule corona data comprises an abundance ratio of two peptides associated with a single protein on two different particles. In some cases, biomolecule corona data comprises sample condition annotations which identify a condition under which the biomolecule was observed. For example, a datapoint may comprise an abundance of a peptide identified from a biological sample, a particle type on which the peptide was identified, and the osmolarity and pH of the sample.

The present disclosure also identifies a number of biomarkers (e.g., proteins) which can be diagnostic for neurological diseases. In some embodiments, the biomarkers (e.g., proteins) are diagnostic for tracking disease progression, such as progression of dementia. In some embodiments, the biomarkers (e.g., proteins) are diagnostic for Alzheimer's disease. In some embodiments, the methods provided herein comprise identifying (e.g., such as via a classifier as described herein) a biomarker (e.g., protein) selected from TABLE 6. TABLE 6 also includes information regarding whether the biomarker is upregulated or downregulated in a neurodegenerative disease described herein (e.g., AD). In some embodiments, the methods comprise identifying at least two biomarkers (or associated peptides or signals) selected from TABLE 6. In some embodiments, the methods comprise identifying at least three biomarkers (or associated peptides or signals) selected from TABLE 6. In some embodiments, the methods comprise identifying at least four biomarkers (or associated peptides or signals) selected from TABLE 6. In some cases, a biomarker (or associated peptide or signal) is annotated with a particle type or condition used for its detection.

TABLE 6

| | | | Regression |
|---|---|---|---|
| Gene | UniProt ID | Name | Coefficient |
| CD34 | P28906 | Hematopoietic progenitor cell antigen | Positive |
| | | CD34 | |
| NAA16 | Q6N069 | N-alpha-acetyltransferase 16, NatA | Positive |
| | | auxiliary subunit | |
| MAPK3 | P27361 | Mitogen-activated protein kinase 3 | Positive |
| SPON2 | Q9BUD6 | Spondin-2 | Negative |
| TUBB2A | Q13885 | Tubulin beta-2A chain | Negative |
| TTYH3 | Q9C0H2 | Protein tweety homolog 3 | Positive |
| N/A | P01717 | Ig lambda chain V-IV region Hil | Positive |
| GPX3 | P22352 | Glutathione peroxidase 3 | Positive |
| DICER1 | Q9UPY3 | Endoribonuclease Dicer | Positive |
| UFC1 | Q9Y3C8 | Ubiquitin-fold modifier-conjugating | Positive |
| | | enzyme 1 | |
| PIN1 | Q13526 | Peptidyl-prolyl cis-trans isomerase NIMA- | Positive |
| | | interacting 1 | |
| COCH | O43405 | Cochlin | Positive |
| RBCK1 | Q9BYM8 | RanBP-type and C3HC4-type zinc finger- | Positive |
| | | containing protein 1 | |
| CHRD | Q9H2X0 | Chordin | Positive |
| ADH1B | P00325 | Alcohol dehydrogenase 1B | Negative |
| ITIH3 | Q06033 | Inter-alpha-trypsin inhibitor heavy chain H3 | Negative |
| TFRC | P02786 | Transferrin receptor protein 1; Transferrin | Negative |
| | | receptor protein 1, serum form | |
| RWDD1 | Q9H446 | RWD domain-containing protein 1 | Positive |
| GRIPAP1 | Q4V328 | GRIP1-associated protein 1 | Positive |
| MGST3 | O14880 | Microsomal glutathione S-transferase 3 | Negative |
| NUB1 | Q9Y5A7 | NEDD8 ultimate buster 1 | Positive |
| CRP | P02741 | C-reactive protein; C-reactive protein(1-205) | Negative |
| HEG1 | Q9ULI3 | Protein HEG homolog 1 | Positive |
| ACSL4 | O60488 | Long-chain-fatty-acid—CoA ligase 4 | Positive |
| MOB3C | Q70IA8 | MOB kinase activator 3C | Positive |
| CSNK1G3 | Q9Y6M4 | Casein kinase I isoform gamma-3 | Positive |
| FRK | P42685 | Tyrosine-protein kinase FRK | Positive |
| CFHR4 | Q92496 | Complement factor H-related protein 4 | Negative |
| N/A | A0A0G2JI36 | N/A | Positive |
| N/A | A0A0G2JIF2 | N/A | Positive |
| N/A | A0A0G2JL56 | N/A | Positive |
| N/A | A0A1W2PQD0 | N/A | Positive |
| HLA-A | Q5SUL5 | N/A | Positive |
| PAK4 | O96013 | Serine/threonine-protein kinase PAK 4 | Positive |
| MAPKAPK2 | P49137 | MAP kinase-activated protein kinase 2 | Positive |
| TRAFD1 | O14545 | TRAF-type zinc finger domain-containing | Positive |
| | | protein 1 | |
| CPVL | Q9H3G5 | Probable serine carboxypeptidase CPVL | Positive |
| BTD | P43251 | Biotinidase | Positive |
| SBDS | Q9Y3A5 | Ribosome maturation protein SBDS | Positive |
| PPIC | P45877 | Peptidyl-prolyl cis-trans isomerase C | Negative |
| INPP4B | O15327 | Type II inositol 3,4-bisphosphate 4- | Positive |
| | | phosphatase | |
| LMCD1 | Q9NZU5 | LIM and cysteine-rich domains protein 1 | Negative |
| MAN1C1 | Q9NR34 | Mannosyl-oligosaccharide 1,2-alpha- | Negative |
| | | mannosidase IC | |
| CPNE1 | Q99829 | Copine-1 | Positive |
| TINAG | Q9UJW2 | Tubulointerstitial nephritis antigen | Positive |
| MFAP5 | Q13361 | Microfibrillar-associated protein 5 | Negative |
| CHST14 | Q8NCH0 | Carbohydrate sulfotransferase 14 | Positive |
| MACF1 | Q9UPN3 | Microtubule-actin cross-linking factor 1, | Negative |
| | | isoforms 1/2/3/5 | |
| BPIFA2 | Q96DR5 | BPI fold-containing family A member 2 | Positive |
| RGS18 | Q9NS28 | Regulator of G-protein signaling 18 | Positive |
| NDNF | Q8TB73 | Protein NDNF | Positive |
| OGN | P20774 | Mimecan | Negative |
| COL4A3BP | Q9Y5P4 | Collagen type IV alpha-3-binding protein | Positive |
| TDRP | Q86YL5 | Testis development-related protein | Positive |
| PSMB2 | P49721 | Proteasome subunit beta type-2 | Negative |
| ARHGEF18 | Q6ZSZ5 | Rho guanine nucleotide exchange factor 18 | Positive |
| RWDD4 | Q6NW29 | RWD domain-containing protein 4 | Positive |
| SCUBE1 | Q8IWY4 | Signal peptide, CUB and EGF-like domain- | Positive |
| | | containing protein 1 | |
| PRKAR2A | P13861 | cAMP-dependent protein kinase type II- | Positive |
| | | alpha regulatory subunit | |
| C1QTNF9 | P0C862 | Complement C1q and tumor necrosis | Negative |
| | | factor-related protein 9A | |
| CLASP2 | O75122 | CLIP-associating protein 2 | Positive |
| SIAE | Q9HAT2 | Sialate O-acetylesterase | Positive |
| TNFRSF14 | Q92956 | Tumor necrosis factor receptor superfamily | Positive |
| | | member 14 | |
| AKR1A1 | P14550 | Alcohol dehydrogenase [NADP(+)] | Positive |
| TMEM33 | P57088 | Transmembrane protein 33 | Negative |
| CCDC92 | Q53HC0 | Coiled-coil domain-containing protein 92 | Positive |
| TTN | Q8WZ42 | Titin | Positive |
| COPZ1 | P61923 | Coatomer subunit zeta-1 | Positive |
| DYNC1H1 | Q14204 | Cytoplasmic dynein 1 heavy chain 1 | Positive |
| SUGT1 | Q9Y2Z0 | Suppressor of G2 allele of SKP1 homolog | Positive |
| FKBP5 | Q13451 | Peptidyl-prolyl cis-trans isomerase | Negative |
| | | FKBP5;Peptidyl-prolyl cis-trans isomerase | |
| | | FKBP5, N-terminally processed | |
| GNA12 | Q03113 | Guanine nucleotide-binding protein subunit | Positive |
| | | alpha-12 | |
| N/A | P06331 | Ig heavy chain V-II region ARH-77 | Positive |
| PTPRE | P23469 | Receptor-type tyrosine-protein phosphatase | Positive |
| | | epsilon | |
| ENO1 | P06733 | Alpha-enolase | Positive |
| TMSB4X | P62328 | Thymosin beta-4; Hematopoietic system | Positive |
| | | regulatory peptide | |
| CYLD | Q9NQC7 | Ubiquitin carboxyl-terminal hydrolase | Positive |
| | | CYLD | |
| CBR1 | P16152 | Carbonyl reductase [NADPH] 1 | Positive |
| MOB3A | Q96BX8 | MOB kinase activator 3A | Positive |
| REPS2 | Q8NFH8 | RalBP1-associated Eps domain-containing | Positive |
| | | protein 2 | |
| IMMT | Q16891 | MICOS complex subunit MIC60 | Negative |
| SGTA | O43765 | Small glutamine-rich tetratricopeptide | Positive |
| | | repeat-containing protein alpha | |
| HLA-DRA | P01903 | HLA class II histocompatibility antigen, DR | Positive |
| | | alpha chain | |
| ETFA | P13804 | Electron transfer flavoprotein subunit alpha, | Negative |
| | | mitochondrial | |
| DDAH2 | O95865 | N(G),N(G)-dimethylarginine | Positive |
| | | dimethylaminohydrolase 2 | |
| MICU1 | Q9BPX6 | Calcium uptake protein 1, mitochondrial | Positive |
| LBP | P18428 | Lipopolysaccharide-binding protein | Negative |
| ENO2 | P09104 | Gamma-enolase | Positive |
| DBI | P07108 | Acyl-CoA-binding protein | Positive |
| TMBIM1 | Q969X1 | Protein lifeguard 3 | Negative |
| PAMR1 | Q6UXH9 | Inactive serine protease PAMR1 | Positive |
| SYAP1 | Q96A49 | Synapse-associated protein 1 | Negative |
| CNDP2 | Q96KP4 | Cytosolic non-specific dipeptidase | Positive |
| EXOC8 | Q8IYI6 | Exocyst complex component 8 | Positive |
| PNLIP | P16233 | Pancreatic triacylglycerol lipase | Positive |
| MTMR12 | Q9C0I1 | Myotubularin-related protein 12 | Positive |
| DBNL | Q9UJU6 | Drebrin-like protein | Positive |
| BTK | Q06187 | Tyrosine-protein kinase BTK | Positive |
| SUCLG1 | P53597 | Succinyl-CoA ligase [ADP/GDP-forming] | Negative |
| | | subunit alpha, mitochondrial | |
| CXorf38 | Q8TB03 | Uncharacterized protein CXorf38 | Positive |
| RPS3A | P61247 | 40S ribosomal protein S3a | Positive |
| CPN2 | P22792 | Carboxypeptidase N subunit 2 | Positive |
| OTUB1 | Q96FW1 | Ubiquitin thioesterase OTUB1 | Positive |
| PARP1 | P09874 | Poly [ADP-ribose] polymerase 1 | Positive |
| PARVB | Q9HBI1 | Beta-parvin | Positive |
| SYNJ1 | O43426 | Synaptojanin-1 | Positive |
| LSM6 | P62312 | U6 snRNA-associated Sm-like protein | Positive |
| | | LSm6 | |
| CARD9 | Q9H257 | Caspase recruitment domain-containing | Positive |
| | | protein 9 | |
| PXDN | Q92626 | Peroxidasin homolog | Negative |
| HDLBP | Q00341 | Vigilin | Positive |
| WDR13 | Q9H1Z4 | WD repeat-containing protein 13 | Positive |
| ADH4 | P08319 | Alcohol dehydrogenase 4 | Negative |
| A1BG | P04217 | Alpha-1B-glycoprotein | Negative |
| PPP2R5E | Q16537 | Serine/threonine-protein phosphatase 2A 56 | Negative |
| | | kDa regulatory subunit epsilon isoform | |
| PLEC | Q15149 | Plectin | Positive |
| COL6A2 | P12110 | Collagen alpha-2(VI) chain | Negative |
| LPL | P06858 | Lipoprotein lipase | Positive |
| CCL23 | P55773 | C-C motif chemokine 23; CCL23(19- | Negative |
| | | 99); CCL23(22-99); CCL23(27- | |
| | | 99); CCL23(30-99) | |
| NUBP2 | Q9Y5Y2 | Cytosolic Fe-S cluster assembly factor | Negative |
| | | NUBP2 | |
| PWP1 | Q13610 | Periodic tryptophan protein 1 homolog | Negative |
| COASY | Q13057 | Bifunctional coenzyme A | Positive |
| | | synthase; Phosphopantetheine | |
| | | adenylyltransferase; Dephospho-CoA kinase | |
| MPO | P05164 | Myeloperoxidase; Myeloperoxidase; 89 kDa | Negative |
| | | myeloperoxidase; 84 kDa | |
| | | myeloperoxidase; Myeloperoxidase light | |
| | | chain; Myeloperoxidase heavy chain | |
| BIN2 | Q9UBW5 | Bridging integrator 2 | Positive |
| KIAA1211 | Q6ZU35 | Uncharacterized protein KIAA1211 | Positive |
| DOCK11 | Q5JSL3 | Dedicator of cytokinesis protein 11 | Positive |
| BRK1 | Q8WUW1 | Protein BRICK1 | Positive |
| TXNDC17 | Q9BRA2 | Thioredoxin domain-containing protein 17 | Positive |
| OLFML2B | Q68BL8 | Olfactomedin-like protein 2B | Negative |
| GLA | P06280 | Alpha-galactosidase A | Positive |
| PTGIR | P43119 | Prostacyclin receptor | Negative |
| RENBP | P51606 | N-acylglucosamine 2-epimerase | Positive |
| ABCB6 | Q9NP58 | ATP-binding cassette sub-family B member | Negative |
| | | 6, mitochondrial | |
| | P01817 | Ig heavy chain V-II region MCE | Positive |
| DDT | P30046 | D-dopachrome decarboxylase | Positive |
| DKK2 | Q9UBU2 | Dickkopf-related protein 2 | Positive |
| RTN4R | Q9BZR6 | Reticulon-4 receptor | Positive |
| PZP | P20742 | Pregnancy zone protein | Negative |
| TNFRSF6B | O95407 | Tumor necrosis factor receptor superfamily | Negative |
| | | member 6B | |

The present disclosure also identifies a number of biomarkers (e.g., proteins) which can be diagnostic for neurological diseases. In some embodiments, the biomarkers (e.g., proteins) are diagnostic for tracking disease progression, such as progression of dementia. In some embodiments, the biomarkers (e.g., proteins) are diagnostic for Alzheimer's disease. In some embodiments, the methods provided herein comprise identifying (e.g., such as via a classifier as described herein) a biomarker (e.g., protein) selected from TABLE 7. In some embodiments, the methods comprise identifying at least two biomarkers (or associated peptides or signals) selected from TABLE 7. In some embodiments, the methods comprise identifying at least three biomarkers (or associated peptides or signals) selected from TABLE 7. In some embodiments, the methods comprise identifying at least four biomarkers (or associated peptides or signals) selected from TABLE 7. In some cases, a biomarker (or associated peptide or signal) is annotated with a particle type or condition used for its detection.

TABLE 7

| | | | Regression |
|---|---|---|---|
| Protein | UniProt ID | Name | Coefficient |
| MBP | P02686 | Myelin basic protein | Negative |
| PLIN4 | Q96Q06 | Perilipin-4 | Negative |
| PKHG1 | Q9ULL1 | Pleckstrin homology domain-containing family G | Negative |
| | | member 1 | |
| EIF1; EIF1B | P41567; O60739 | Eukaryotic translation initiation factor | Negative |
| | | 1; Eukaryotic translation initiation factor 1b | |
| CRBG3 | Q68DQ2 | Very large A-kinase anchor protein | Negative |
| ANTR1 | Q9H6X2 | Anthrax toxin receptor 1 | Negative |
| PUR6 | P22234 | Bifunctional phosphoribosylaminoimidazole | Negative |
| | | carboxylase/phosphoribosylaminoimidazole | |
| | | succinocarboxamide synthetase | |
| LUM | P51884 | Lumican | Negative |
| C1QT1 | Q9BXJ1 | Complement C1q tumor necrosis factor-related | Negative |
| | | protein 1 | |
| ENOB | P13929 | Beta-enolase | Negative |
| DKK3 | Q9UBP4 | Dickkopf-related protein 3 | Negative |
| GPSM3 | Q9Y4H4 | G-protein-signaling modulator 3 | Negative |
| SYAC | P49588 | Alanine—tRNA ligase, cytoplasmic | Negative |
| HSP74 | P34932 | Heat shock 70 kDa protein 4 | Positive |
| MICA1 | Q8TDZ2 | [F-actin]-monooxygenase MICAL1 | Positive |
| APOD | P05090 | Apolipoprotein D | Positive |
| LRC32 | Q14392 | Transforming growth factor beta activator | Positive |
| | | LRRC32 | |
| CSPG2 | P13611 | Versican core protein | Positive |
| OSTCN | P02818 | Osteocalcin | Positive |

In some embodiments, the present disclosure identifies a number of biomarkers (e.g., proteins) which can be indicators of progression of neurodegenerative diseases. In some embodiments, the biomarkers (e.g., proteins) are indicative of progression (e.g., or delay in progression) of dementia. In some embodiments, the methods provided herein comprise identifying (e.g., such as via a classifier as described herein) a biomarker (e.g., protein) selected from TABLE 7. In some embodiments, the methods comprise identifying at least two biomarkers (or associated peptides or signals) selected from TABLE 8. In some embodiments, the methods comprise identifying at least three biomarkers (or associated peptides or signals) selected from TABLE 8. In some embodiments, the methods comprise identifying at least four biomarkers (or associated peptides or signals) selected from TABLE 8. In some cases, a biomarker (or associated peptide or signal) is annotated with a particle type or condition used for its detection.

TABLE 8

| | | | Regression |
|---|---|---|---|
| Protein | UniProt ID | Name | Coefficient |
| CRISPLD2 | Q9H0B8 | Cysteine-rich secretory protein LCCL | Positive |
| | | domain-containing 2 | |
| CLNS1A | P54105 | Methylosome subunit pICln | Positive |
| BLVRB | P30043 | Flavin reductase (NADPH) | Positive |
| SMYD5 | Q6GMV2 | Histone-lysine N-trimethyltransferase | Positive |
| | | SMYD5 | |
| PRPS1 | P60891 | Ribose-phosphate pyrophosphokinase 1 | Positive |
| SELENBP1 | Q13228_Q13228.4 | Methanethiol oxidase | Positive |
| OXSR1 | O95747 | Serine/threonine-protein kinase OSR1 | Positive |
| VGF | O15240 | Neurosecretory protein VGF | Negative |
| GOLPH3 | GOLPH3 | Golgi Phosphoprotein 3 | Negative |

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” “less than or equal to,” or “at most” precedes the first numerical value in a series of two or more numerical values, the term “no more than,” “less than” or “less than or equal to,” or “at most” applies to each of the numerical values in that series of numerical values. For example, less than or equal to 3, 2, or 1 is equivalent to less than or equal to 3, less than or equal to 2, or less than or equal to 1.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

### Numbered Embodiments

Provided herein are numbered embodiments 1-37.

Embodiment 1. A method of assessing a likelihood of Alzheimer's disease (AD), the method comprising:
assaying a biofluid sample to obtain a data set comprising protein or peptide information;
identifying, from the data set, an abundance of one or more biomarkers provided in Table 6 or Table 7; and
assessing the likelihood of AD, based on the abundance of the one or more biomarkers in the biofluid sample.

Embodiment 2. The method of embodiment 1, wherein the one or more biomarkers comprise one or more of MBP, PLIN4, PKHG1, EIF1:EIF1B, CRBG3, ANTR1, PUR6, LUM, C1QT1, ENOB, DKK3, GPSM3, SYAC, HSP74, MICA1, APOD, LRC32, CSPG2, and OSTCN.

Embodiment 3. The method of embodiment 1 or 2, wherein the one or more biomarkers comprise one or more of MBP, PLIN4, PKHG1, EIF1:EIF1B, CRBG3, ANTR1, PUR6, LUM, C1QT1, ENOB, DKK3, GPSM3, SYAC, HSP74, MICA1, LRC32, CSPG2, and OSTCN.

Embodiment 4. A method of diagnosing Alzheimer's disease, the method comprising:
contacting a biofluid sample from a subject with one or more physicochemically distinct particles to form a plurality of biomolecule coronas;
obtaining a data set comprising protein or peptide information from the plurality of biomolecule coronas;
identifying, from the data set, an abundance of one or more biomarkers provided in Table 6 or Table 7; and
using a classifier to identify the biofluid sample as being indicative of a biological state comprising a healthy state or Alzheimer's disease (AD), based on the abundance of the one or more biomarkers in the data set.

Embodiment 5. The method of embodiment 4, wherein the one or more biomarkers comprise one or more of MBP, PLIN4, PKHG1, EIF1:EIF1B, CRBG3, ANTR1, PUR6, LUM, C1QT1, ENOB, DKK3, GPSM3, SYAC, HSP74, MICA1, APOD, LRC32, CSPG2, and OSTCN.

Embodiment 6. The method of embodiment 4 or 5, wherein the one or more biomarkers comprise MBP, PLIN4, PKHG1, EIF1:EIF1B, CRBG3, ANTR1, PUR6, LUM, C1QT1, ENOB, DKK3, GPSM3, SYAC, HSP74, MICA1, LRC32, CSPG2, and OSTCN.

Embodiment 7. The method of any one of embodiments 4-6, wherein the one or more biomarkers comprise one or more of MBP and OSTCN.

Embodiment 8. The method of any one of the preceding embodiments, wherein the one or more biomarkers comprise two or more biomarkers.

Embodiment 9. The method of any one of embodiments 1-6, wherein the one or more biomarkers comprise three or more biomarkers.

Embodiment 10. The method of any one of embodiments 1-6, wherein the one or more biomarkers comprise four or more biomarkers.

Embodiment 11. The method of any one of the preceding embodiments, wherein the one or more biomarkers further comprise an additional biomarker comprising pTau (e.g., pTau-181 or pTau-217).

Embodiment 12. The method of any one of the preceding embodiments, wherein the classifier comprises a machine learning algorithm.

Embodiment 13. The method of embodiment 12, wherein the machine learning algorithm comprises a logistic regression-based machine learning model.

Embodiment 14. A method for assessing a likelihood of dementia progression, the method comprising:
assaying a biofluid sample to obtain a data set comprising protein or peptide information; identifying, from the data set, an abundance of one or more biomarkers; and
assessing a rate of dementia progression in the biofluid sample, based on the abundance of the one or more biomarkers.

Embodiment 15. A method for assessing a likelihood of dementia progression, the method comprising:
contacting a biofluid sample from a subject with one or more physicochemically distinct particles to form a plurality of biomolecule coronas;
obtaining a data set comprising protein or peptide information from the plurality of biomolecule coronas;
identifying, from the data set, an abundance of one or more biomarkers; and
using a classifier to track a rate of dementia progression in the biofluid sample, based on the abundance of the one or more biomarkers in the data set.

Embodiment 16. The method of embodiment 14 or 15, wherein the one or more biomarkers comprise one or more of CRISPLD2, CLNS1A, BLVRB, SMYD5, PRPS1, SELENBP1, OXSR1, VGF, and GOLPH3.

Embodiment 17. The method of embodiment 16, wherein CRISPLD2, CLNS1A, BLVRB, SMYD5, PRPS1, SELENBP1, OXSR1, or a combination thereof are associated with the biofluid sample having an increased rate of dementia progression.

Embodiment 18. The method of embodiment 16 or 17, wherein the CRISPLD2, CLNS1A, or a combination thereof are associated with the biofluid sample having an increased rate of dementia progression.

Embodiment 19. The method of embodiments 17 or 18, wherein the increased rate of dementia progression is associated with a shorter time to clinical dementia rating global (Cargo) increase.

Embodiment 20. The method of embodiment 16, wherein GOLPH3, VGF, or a combination thereof are associated with the biofluid sample having a decreased rate of dementia progression.

Embodiment 21. The method of embodiment 20, wherein GOLPH3 is associated with the biofluid sample having a decreased rate of dementia progression.

Embodiment 22. The method of embodiment 20 or 21, wherein the decreased rate of dementia progression is associated with a delay in CDRg increase.

Embodiment 23. The method of any one of embodiments 15-22, wherein the classifier comprises time-to-event analysis.

Embodiment 24. The method of any one of embodiments 15-23, wherein the classifier comprises Cox proportion hazards (CPH) models, Cox time-varying (CTV) regression models, or a combination thereof.

Embodiment 25. The method of embodiment 24, wherein the classifier comprises CTV regression models.

Embodiment 26. The method of any one embodiments 4-25, wherein the physicochemically distinct particles comprise lipid particles, metal particles, silica particles, or polymer particles.

Embodiment 27. The method of any one of embodiments 4-26, wherein the physicochemically distinct particles comprise polystyrene particles, magnetizable particles, dextran particles, silica particles, dimethylamine particles, carboxylate particles, amino particles, benzoic acid particles, or agglutinin particles.

Embodiment 28. The method of any one of the preceding embodiments, wherein contacting comprises incubating the biofluid sample with the one or more physicochemically distinct particles.

Embodiment 29. The method of embodiment 28, wherein contacting comprises incubating the biofluid sample with the one or more physicochemically distinct particles for about 1 hour.

Embodiment 30. The method of any one of embodiments 4-29, wherein obtaining the data set comprises detecting proteins of the biomolecule coronas by mass spectrometry, chromatography, liquid chromatography, high-performance liquid chromatography, solid-phase chromatography, a lateral flow assay, an immunoassay, an enzyme-linked immunosorbent assay, a western blot, a dot blot, or immunostaining, or a combination thereof.

Embodiment 31. The method of any one of embodiments 4-30, wherein obtaining the data set comprises detecting the proteins of the biomolecule coronas by mass spectrometry.

Embodiment 32. The method of any one of embodiments 4-31, wherein obtaining the data set comprises detecting the proteins of the biomolecule coronas by liquid chromatography mass spectrometry (LC-MS).

Embodiment 33. The method of any one of embodiments 4-32, wherein obtaining the data set comprises measuring a readout indicative of the presence, absence, or amount of proteins of the biomolecule coronas.

Embodiment 34. The method of any one of the preceding embodiments, wherein the biofluid sample comprises a blood sample, a serum sample, or a plasma sample.

Embodiment 35. The method of any one of the preceding embodiments, wherein the biofluid sample comprises a blood sample that has had red blood cells removed.

Embodiment 36. The method of any one of the preceding embodiments, wherein the biofluid is plasma.

Embodiment 37. The method of any one of the preceding embodiments, wherein the one or more physicochemically distinct particles comprise 2 or more physicochemically distinct particles.

### EXAMPLES

The following examples are illustrative and non-limiting to the scope of the compositions, devices, systems, kits, and methods described herein.

### Example 1—Particle-Based Detection of Alzheimer's Disease and Related Dementias Methods

The cohort consisted of about 1005 subjects whom at least one plasma had been collected between 2008 and 2019. This is a longitudinal observational study spanning the continuum of normal aging to ADRD. Annual standardized assessments included a general and neurological exam, a semi-structured interview with the participant and/or informant to record cognitive symptoms with a Clinical Dementia Rating scale (CDR Dementia Staging Instrument), a battery of neuropsychological tests and other instruments of the National Alzheimer's Coordinating Center (NACC) Uniform Dataset (UDS). Blood was collected from all consenting subjects.

Cognitive status and clinical syndromes diagnosis were determined at each visit by a consensus team after a detailed examination and review of all available information according to the 2011 NIA-AA diagnostic criteria for MCI and AD. Many participants had autopsy, imaging, CSF, and/or plasma biomarkers in affiliated protocols. Disease diagnosis (AD or other diseases) was further informed by these data when available. Participant clinical data used in the analyses here include age, sex, race, ethnicity, years of education, and clinical dementia rating global (CDRg) scores taken concurrently with sample collection. Additional biomarker data available on almost all cases included apolipoprotein e (APOE) genotype as well as plasma phospho-tau 181 (pTau181), glial fibrillary acidic protein (GFAP) and neurofilament-light (NfL). Plasma biomarkers were measured using ultrasensitive MSD S-PLEX electrochemiluminescence immunoassay kits (Meso Scale Discovery, Rockville, MD), as previously described. Participant summary statistics are in TABLE 9.

TABLE 9

| | | | No | | | |
|---|---|---|---|---|---|---|
| | Alzheimer's | | Neurodegenerative | | | |
| Characteristics | Disease | | Disease | | Other Dementias | |
| Number of subjects (N) | 379 | | 240 | | 387 | |
| Number of visits [median | 1 | (1, 6) | 2 | (1, 6) | 1 | (1, 6) |
| (min, max)] | | | | | | |
| Age at 1^{st} visit [Mean (SD)] | 74.5 | (9.4) | 66.7 | (11.6) | 71.4 | (9.9) |
| Female (%) | 190 | (50.1) | 166 | (69.2) | 212 | (54.8) |
| College educated [N (%)] | 264 | (69.7) | 165 | (68.8) | 253 | (65.4) |
| APOE ε4 carriers [N (%)] | 185 | (48.4) | 55 | (22.9) | 82 | (21.2) |
| Last Draw CDR sum of | 5.0 | (4.5) | 0.2 | (0.5) | 3.9 | (4.6) |
| boxes [Mean (SD)] | | | | | | |
| Last Draw MMSE [Mean | 18.0 | (8.2) | 26.6 | (2.8) | 21.5 | (7.2) |
| (SD)] | | | | | | |
| Last Draw pTau-181 (pg/mL) | 4.7 | (3.3) | 2.5 | (1.3) | 2.5 | (1.4) |
| [Mean (SD)] | | | | | | |
| Last Draw NfL (pg/mL) | 319.4 | (213.0) | 180.2 | (106.7) | 326.7 | (322.9) |
| Mean (SD)]¹ | | | | | | |
| Last Draw GFAP (pg/mL) | 146.9 | (125.5) | 80.5 | (44.2) | 99.2 | (66.1) |
| [Mean (SD)] | | | | | | |

### Sample Preparation

Plasma samples used in this study had been collected between 2008 and 2019. Samples were collected in K2EDTA tubes, centrifuged at 2000 g or 5 min, frozen in low retention polypropylene cryovials within 4 hours of collection and stored at −80° C. until use.

Plasma from 1,786 individual samples (including subsequent plasma collection samples from the same individuals) and a plasma control sample (PC6), consisting of pooled citrate phosphate dextrose anticoagulant plasma from 15 healthy individuals, were processed with the Proteograph XT Assay Kit. Plasma tubes containing 240 μL of plasma were loaded onto the SP100 Automation Instrument for sample preparation to generate purified peptides for LC-MS analysis. The samples were incubated to form each of the two, physicochemically distinct nanoparticle (NP) suspensions for protein corona formation. Samples (40 samples/plate; 38-39 individual plasma samples and 1-2 PC6 samples) were automatically plated, including process controls, digestion control, and MPE peptide clean-up control. After a one-hour incubation, leveraging the paramagnetic property of NPs, NP-bound proteins were captured using magnetic isolation. A series of gentle washes removed nonspecific and weakly bound proteins. This process results in a highly specific and reproducible protein corona. Protein coronas are denatured, reduced, alkylated, and digested with Trypsin/Lys-C to generate tryptic peptides for LC-MS analysis. All steps were performed in a one-pot reaction directly on the NPs. The in-solution digestion mixture was then desalted and all detergents were removed using a solid phase extraction and positive pressure (MPE) system on SP100 Automation Instrument. Clean peptides were eluted in a high-organic buffer within a deep-well collection plate and quantified. Equal volumes of peptide elution were dried down in a SpeedVac (3 hours-overnight), and the resulting dried peptides were stored at −80° C. or directly analyzed by liquid-chromatography mass-spectroscopy (LC-MS). Quantified peptides were reconstituted to a final concentration of 0.06 μg/μL in Proteograph XT Assay Kit Reconstitution Buffer.

### LC-MS Analysis

8 μL of the reconstituted peptides were loaded on an Acclaim PepMap 100 C18 (0.3 mm ID×5 mm) trap column and then separated on an Ultimate 3000 HPLC System and a 50 cm μPAC HPLC column (Thermo Fisher Scientific) at a flow rate of 1 μL/minute using a gradient of 5-25% solvent B (0.1% FA, 100% ACN) in solvent A (0.1% FA, 100% water) over 22 minutes, resulting in a 33-minute total run time. For the MS analysis on the Thermo Fisher Scientific Orbitrap Exploris 480 MS, 480 ng of material per NP was analyzed in DIA mode using 10 m/z isolation windows from 380-1000 m/z. MS1 scans were acquired at 60 k resolution and MS2 at 30 k resolution.

### Spectral Library Generation

An MS-only workflow was used that combines GPF and DIA LC-MS, saving significant experiment time while maintaining high data completeness and reproducibility. This strategy generated a chromatogram spectral library with GPF deep scanning experiments, consisting of staggered m/z window analysis of the pooled peptides left over from Proteograph XT Assay plates by pooling up to 5 μL of tryptic peptides left for each sample in the plate into separate pools for each NP suspension. Six DIA LC-MS injections of 10 μL each containing a peptide concentration of 0.06 ug/μL from each NP pool were analyzed. The six injections covered mass over charge (m/z) ranges of 400-500 m/z, 500-600 m/z, 600-700 m/z, 700-800 m/z, 800-900 m/z, and 900-1000 m/z, with each injection having 50 staggered windows covering 4 m/z. MS1 was run in 60K resolution and MS2 was run in 30K resolution on another Orbitrap Exploris 480 MS with similar chromatographic setup (LC, trap, and column). A library-free search of the DIA LC-MS data was performed using DIA-NN 1.8.1 to create the empirically corrected GPF library.

### Data Analysis and Protein Representation

All MS files were developed to run DIA-NN 1.8.1 with a GPF library search. All identifications are reported at 1% FDR. Panel protein representations integrated nanoparticle: precursor representations with MaxLFQ.

### Functional Annotation Enrichment and Differential Expression Analysis

To determine how the biological variables in this cohort correlate with protein abundances comparing profiles across the 4,007 protein groups and 1,786 plasma samples, a linear mixed-effects model was trained (LMM; lme4) with ProteinIntensity˜Diagnosis+Diagnosis: (Age+Sex+Education+globalCDR+ApoE_score)+Education+Sex+Age+Sample Variation+ (1|Collection Year)+(1|NP: AssayPlate), where Diagnosis contains 3 levels of AD, other dementia, and no neurodegenerative disease, and ApoE score is calculated as (−0.5*n of e2 alleles+1*n of e4 alleles). SampleVariation is a technical variable that accounts for variabilities in the samples resulting from differences in NP:protein interactions that are due to variations in sample collection. The variable variable for the median fold-change of proteins is annotated as “Nucleolus” for each plasma sample and NP. Collection Year is included as a random effect to account for sample variability based on the year of sample collection, and NP and assay plates associated with the NP is accounting for sample preparation variabilities. The missing protein intensities are imputed for the NP that has the lowest number of missingness across all samples, and in the case of equal missingness the NP with higher protein intensity is picked for imputation. The imputation is done by sampling 3 times from a shifted normal distribution for that feature with mean shift=−1.8 and width=0.315.

To determine functional annotations associated with the LMM results, annotations were matched with UniProt identifiers and enrichments calculated based on the coefficient distributions using the R AnnoCrawler package and implementation of 1D annotation enrichment.

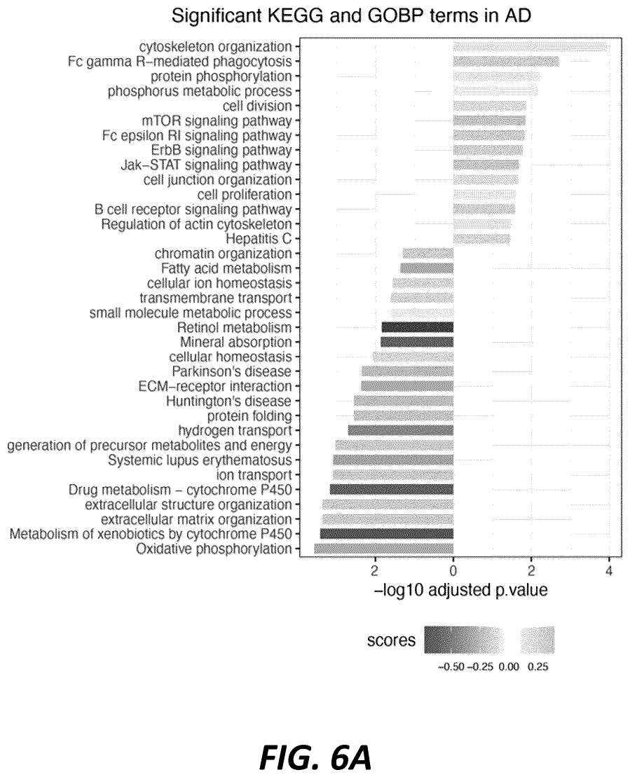

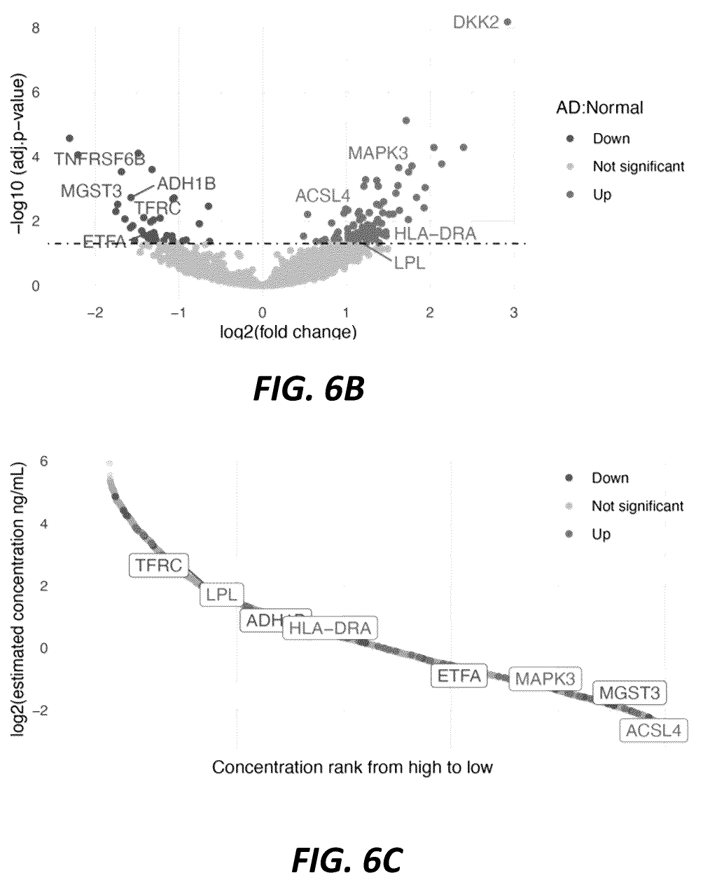

To indicate how proteins are differentially abundant in AD cases in contrast to the group without neurodegenerative disease, the LMM coefficients, where Diagnosis=AD, were plotted against the negative log 10 transformed p-values where the p-values are corrected for multiple testing according to the Benjamini-Hochberg method (FIG. 6B).

### Diagnostic Cohort and Machine Learning Diagnostic Model

A Diagnostic cohort was established using the final draw from each sample for the purpose of evaluating the use of protein biomarkers for determining AD status and identifying AD related proteins. These data were then further restricted to only those subjects which diagnosed as “AD” or “No Neurodegenerative Disease.” Cases where the diagnosis was made on the basis of pTau-181 were excluded to avoid confounding of diagnostic state. The final set of samples included 141 AD subjects, and 217 No Neurodegenerative Disease (referred to as “Normal”) subjects.

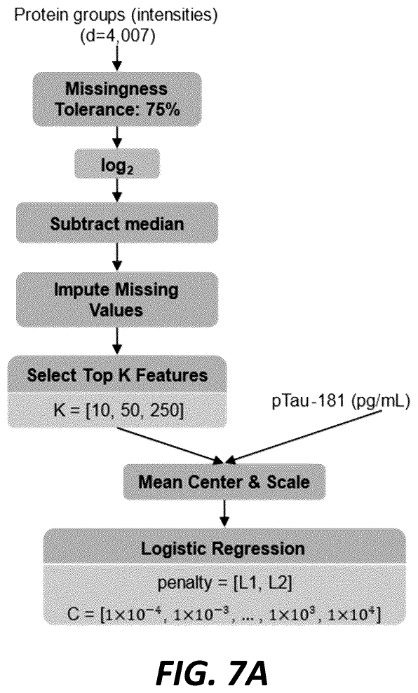

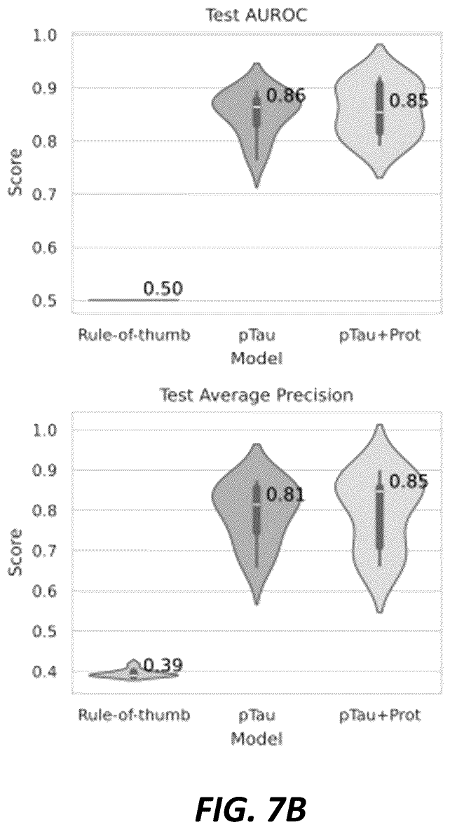

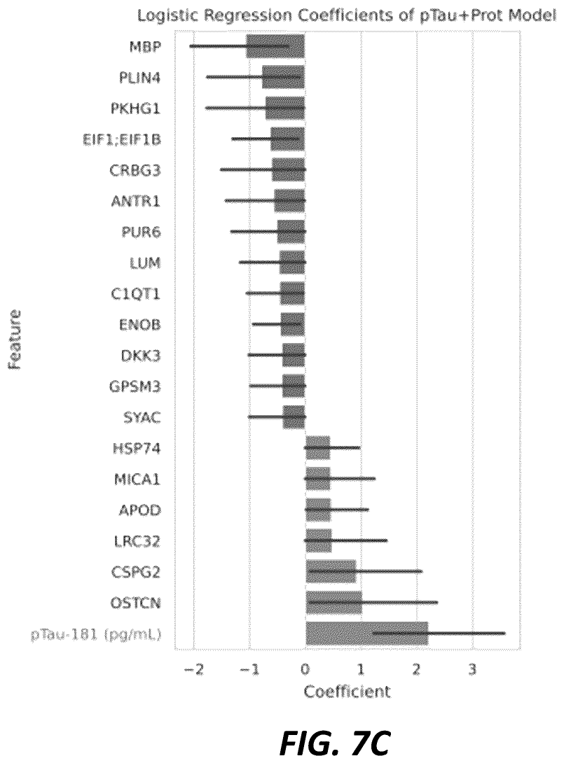

A machine learning model was developed to classify AD and Normal subjects based on their plasma proteomics features from LC-MS, in addition to pTau-181 concentration. The logistic regression model includes a preprocessing pipeline for the proteomics features that appropriately handles missing data, imputation, normalization, and feature selection (FIG. 7A). Protein intensities are first filtered by missingness, keeping only features that have a missing rate of less than or equal to 75% among the training samples. The features are normalized by taking the logarithm and subtracting each feature's median. Any remaining missing values are imputed by sampling from a shifted normal distribution for that feature with mean shift=−1.8 and width=0.3¹⁵. The top-K features are then identified by computing the ANOVA F-score between the labels and features, and keeping the K highest scoring features. Finally, pTau-181 (pg/mL) is added as a feature and the whole set of features is mean centered and unit variance scaled, before passing to a penalized logistic regression classifier.

The model described above has hyperparameters (K for feature selection, and penalty kind and amount for logistic regression) that must be tuned, and logistic regression coefficients that must be fit to the data. To avoid overfitting, nested cross-validation strategy was adopted. 10 outer folds are created. For each of these 10 folds, the other 9 are taken as the training set. This training set is then further split in an inner hyperparameter tuning stage, where 80% of it is used to fit a model for each possible hyperparameter setting, and the other 20% is a validation set used to evaluate the hyperparameter setting. The best hyperparameter setting (highest area under the receiver operator characteristic curve (AUROC)) is then refit on the full 9-fold training set, and a test score is computed on the test fold.

### Time-to-Event Analysis

Cox proportional hazards (CPH) and Cox time-varying (CTV) regression models were built to determine the association of each protein group with the time to CDRg increase (the event) from either CDRg of 0.0 or CDRg of 0.5. Subjects who showed an increase from baseline (0.0 to 0.5 or 0.5 to 1) after a minimum of 1 post-draw visit were labeled as E=1 while those that did not show an increase for their observation time and for at least 3 years were categorized as E=0 (censored). With these criteria, the original dataset was subset in the CDRg baseline 0.0 model with 300 subjects and 540 biosamples (n=145 subjects with multiple draws) and the CDRg baseline of 0.5 model had 391 subjects and 684 biosamples (n=209 subjects with multiple draws). 70 subjects were in the models for both baselines. TABLE 10 describe three sets of Cox regression model types in this study.

TABLE 10

| | | | Uses | | |
|---|---|---|---|---|---|
| | Model | Patient | Delayed | | |
| | type | samples | Entry | Timescale | Purpose |
| Model 1 | CTV | All before event | Yes | Age | Represent multiple |
| | | | | | blood draws and |
| | | | | | time-varying |
| | | | | | protein levels |
| Model 2 | CPH | Last before | Yes | Age | Comparison with |
| | | event | | | CTV model |
| Model 3 | CPH | Last before | No | Follow-up | Check of PH |
| | | event | | time | assumption and |
| | | | | | generation of |
| | | | | | survival curves |

The time-varying model (Model 1) maximizes the data available from subjects with multiple blood draws and represents proteins as a time-varying covariate. Models 2 and 3 only used the last available blood draw before an event. The last draw model showed a greater correlation with the CTV model than the first draw model and therefore the last draw was the basis for assessing the proportional hazards assumption and survival curve generation in Model 3. Models 1 and 2 used age as time-scale given the importance of age in dementia. Both models also accounted for delayed entry, where entry time is the age of earliest draw at a subject's baseline since the MADRC cohort is an observational study with an open cohort. Model 3 did not use age as time-scale and used age as a covariate instead. All models assessed the association of each protein group while controlling for the subject-level covariates sex, education, ApoEe4 risk score (−0.5*n of e2 alleles+1*n of e4 alleles) and technical-level covariates that contributed to variation of the protein group itself, including plate group identifier, collection year, and nucleolus score. Models were created for a protein group only if there was a minimum completeness of 25%; samples with missing values for a protein group were not considered in that model. The intensity values of each protein group were log 2 transformed and standardized. Since one model was built for each protein group, multiple hypothesis testing was accounted for by applying Benjamini-Hochberg adjustment to nominal p-values. In partial regression coefficient plots, the levels of protein group features are shown as z-scores. The python package lifelines was used to create the Cox models.

### Results and Discussion

### Study Design and Protein Quantification Metrics

Samples were collected from an observational study of a group of individuals with or without cognitive impairment in a longitudinal fashion with data collected on a nearly annual basis. Data include cognitive tests and blood collection (average 6.2±standard deviation 3.80 visits per subject), although proteomics was obtained for only a subset of blood draws (1.8±1.04 blood draws per subject). Final primary disease diagnoses were also provided along with a method of determination such as neuropathology, molecular neuroimaging, CSF and/or plasma biomarker. Plasma samples were processed for deep LC-MS proteomics. In the analysis of 1,786 plasma samples, 4,007 protein groups (3,692 for those in at least 25% of samples) were identified and 36,259 peptides using the GPF library.

### Differential Expression of Proteins in Alzheimer's Patients

To investigate the biological pathways involved in AD and the identification of proteins that are differentially abundant between AD and control samples, all 1786 plasma samples were analyzed, including 498 plasma samples from subjects without neurodegenerative disease, 653 plasma samples from subjects with AD, and 635 plasma samples from subjects with other types of dementia.

A linear mixed model was used describing the normalized intensities of all identified proteins as a function of diagnosis, age, sex, education, global CDR score, APOE alleles, and technical variables such as NPs, assay plates, sample collection year, and plasma protein composition. The resulting coefficients for DemD×AD from this model were then used in a 1D annotation enrichment analysis to evaluate how these biological variables are differentially associated with functional annotations. FIG. 6A shows the biological processes that are significantly dysregulated in AD. For example, oxidative phosphorylation is shown to be downregulated (Enrichment score=−0.34). Mitochondrial dysfunction, associated with a decrease in neuronal ATP levels, may be a characteristic feature of AD. This dysfunction may be partly due to the overproduction of reactive oxygen species (ROS), leading to oxidative stress and damage to mitochondrial function. In AD, this may result in compromised oxidative phosphorylation, leading to neuronal cell death. The extracellular matrix (ECM) is another biological process that has been shown to be potentially dysregulated in AD (FIG. 6A). Dysregulation of ECM may play a role in its pathogenesis, and it is involved in various aspects of AD, including synaptic transmission, amyloid-b plague generation and degeneration, tau-protein production, oxidative stress response, and inflammatory response. Alterations in ECM components may affect the stability of perineuronal nets, impacting the clearance of amyloid-β and the production of neurofibrillary tangles. Signaling pathways such as mTOR, ErbB, and Jak-STAT that are shown to be upregulated in AD subjects in this dataset, are known pathways related to the pathogenesis of AD (FIG. 6A). In addition, pathways related to Parkinson's disease (PD) and Huntington's disease (HD) are shown to be significantly dysregulated. Although each of these diseases has its unique pathophysiological mechanisms, they may share some common mechanisms, including misfolding and aggregation of beta-amyloid and a-synuclein, leading to neuronal apoptosis.

To gain insights into which proteins are differentially abundant in the plasma of AD patients compared the control group, a differential expression analysis was performed using the same linear mixed model as that above. This analysis resulted in 138 differentially abundant proteins of which 38 are down-regulated proteins and 100 up-regulated proteins (FIG. 6B). For instance, MAPK3, one of the up-regulated proteins in AD subjects in this dataset, is known to play a crucial role in AD. The MAPK pathways, including extracellular signal-regulated kinase (ERK), c-Jun N-terminal kinase (JNK), and p38 pathways, are activated in neurons vulnerable to AD. The MAPK pathways may be linked to significant pathological processes in AD, such as tau phosphorylation, amyloid-beta deposition, and amyloid-beta protein precursor functioning. ACLS4 is another upregulated protein in AD subjects. ACLS4 may be involved in the regulation of synaptic function and neuronal signaling and previous studies have shown that its level is significantly increased in AD patients. DKK2 which is the most up-regulated amongst AD subjects in this dataset, is an inhibitor of the Wnt signaling pathway, which is known to be crucial for cognitive function, and its upregulation may contribute to reduced WnT signaling in AD (FIG. 6B).

MGST3 which is one of the downregulated proteins in AD subjects (FIG. 6B), is known to be significantly associated with hippocampus size and may be linked to neurodegenerative disorders associated with reduced hippocampus volume such as AD, PD, and HD. ADH1B which has a protective association in our data, may suppress Aβ-induced neuron apoptosis, and mutations in its gene may be associated with the development of AD.

To investigate how abundant these signature proteins are in blood plasma, the identified proteins in our cohort were mapped to the Human Plasma proteome (HPPP) Database. FIG. 6C shows that the dysregulated proteins are distributed across the dynamic range of the plasma proteome with some of the highlighted proteins such as MAPK3, MGST3, and ACSL4 being at the lower abundance range. In this regard, the finding of their differential expression in AD is noteworthy because these proteins would have been invisible to measure accurately in a large cohort without the use of the methods described herein.

### Biomarker-Based Classification of Alzheimer's Patients

It was investigated whether there is a multimarker signature of AD that can be identified from the proteomics data. To this end, a subset of samples was focused on, using the last draw from each subject that has at least two clinical visits. These data were restricted to include only those subjects diagnosed as “AD” or “No Neurodegenerative Disease” and took care to ensure that there was no confounding information through inclusion of cases where the diagnostic status is based on biomarkers to be evaluated. The final set of samples included 141 AD subjects, and 217 No Neurodegenerative Disease (referred to as “Normal”) subjects (Methods).

A logistic regression-based machine learning model was developed to classify AD versus Normal using both pTau-181 concentration and LC-MS proteomics features and evaluate it using nested cross validation (Methods, Machine Learning Diagnostic Model). Since the dataset is imbalanced (AD is the minority class), the average precision (average positive predictive value) in addition to AUROC is also reported (FIG. 7B). The model was compared to a rule-of-thumb classifier that would just report the prior class distribution as the predicted probability of AD, in addition to comparing to the value of pTau-181 concentration alone.

The fitted models can be analyzed to determine which input features were most influential in classifying AD and Normal (e.g., healthy) patients. The average of the logistic regression coefficients across the 10 models (from the 10 folds) was computed, and the top 20 (based on absolute value) are reported in FIG. 7C. While pTau-181 concentration was the most influential feature, other noteworthy proteomics features also had large coefficients. Myeloid basic protein (MBP) was associated with Normal subjects (negative coefficient, protective), and may act as an amyloid β-protein (Aβ) chaperone and may be an inhibitor of accumulation of Aβ fibrils. The relation of MBP to AD pathology is still an open area of research. In addition, the models gave a large positive coefficient to osteocalcin (OSTCN), a marker of processes involved in osteoporosis such as bone remodeling and anabolism, and may have some comorbidity of AD and osteoporosis. Apolipoprotein D (APOD) also had a large positive coefficient and may have increased levels in AD where it plays a neuroprotective role against oxidative stress.

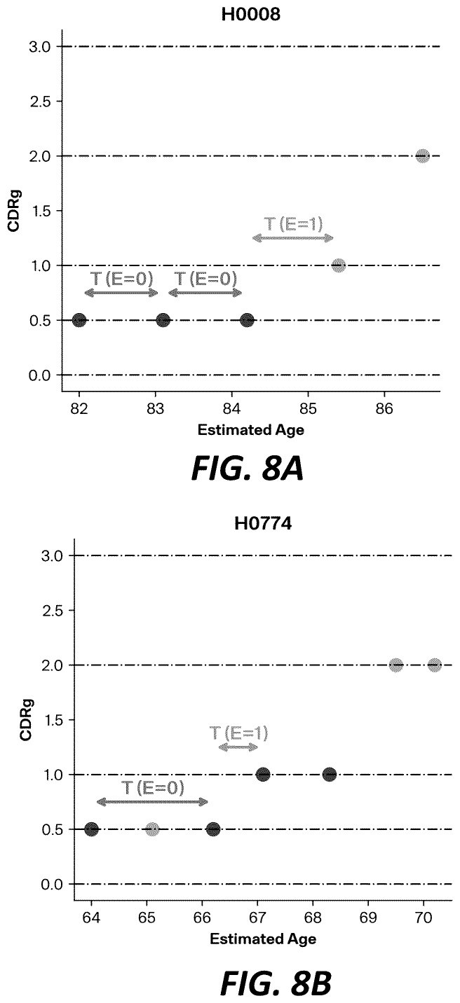

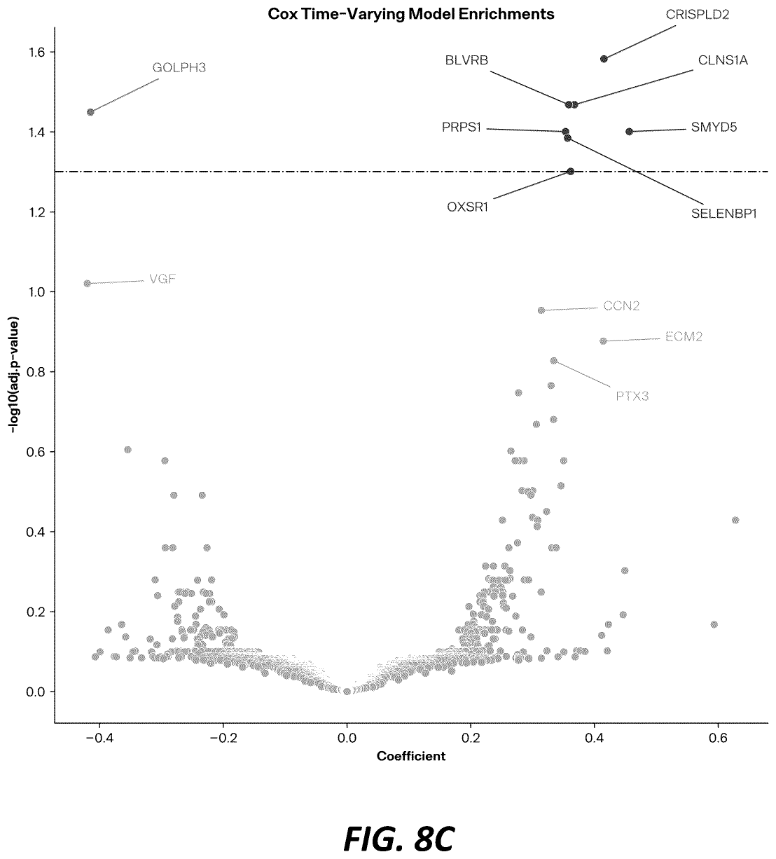

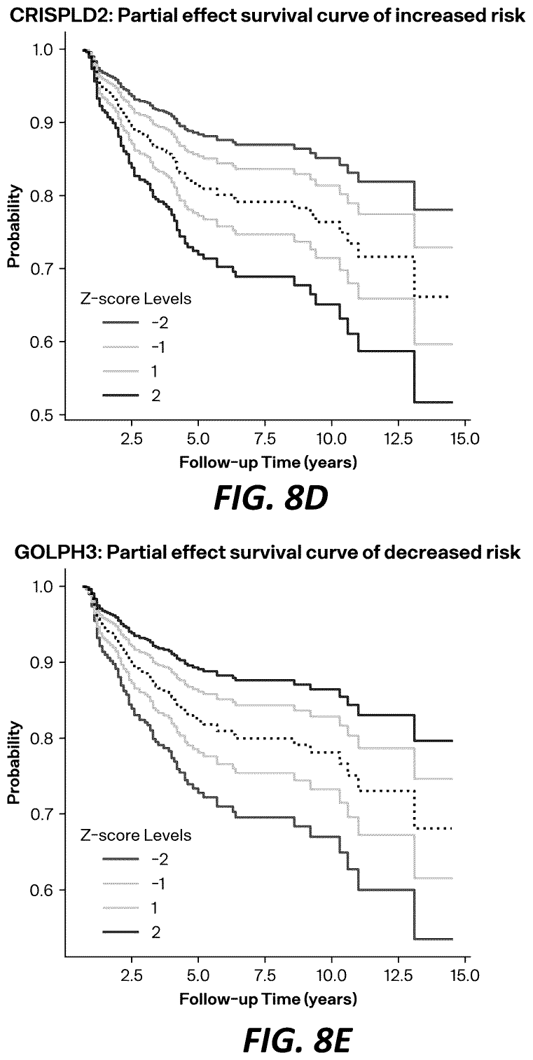

To determine the association of protein groups to dementia progression, multiple Cox regression models were employed where the time to CDRg increase was assessed. The primary model (Model 1) is a Cox time-varying model that represents delayed entry, due to open cohort enrollment, right-censored events, and age as timescale. The time-varying component of this model allows the protein expression to be represented over time when multiple draws are available for a subject (FIGS. 8A-B) and for the proportional hazard assumption to be relaxed. It was reasoned that cognitively normal subjects (CDRg of 0.0) would have different rates of dementia progression than those already showing mild cognitive impairment (MCI, CDRg of 0.5). Therefore one model was built for particonats with a baseline CDRg=0 and another for baseline CDRg=0.5. The distribution in subjects' final diagnoses for non-neurodegenerative, AD, and other dementias was 179, 43, and 78 subjects respectively in the CDRg 0.0 cohort and 40, 164, and 188 subjects, respectively, in the CDRg 0.5 cohort.

The CTV models for events greater than 0.0 model had no protein groups significantly associated with time-to-CDRg increase. However, the CTV models for events greater than 0.5 identified eight protein groups with coefficients that were significantly associated (p-adj<0.05 after BH correction) (FIG. 8B). Seven protein groups had positive coefficients indicating that elevated levels may implicate a shorter time-to-CDRg increase: CRISPLD2 (Q9H0B8), CLNS1A (P54105), BLVRB (P30043), SMYD5 (Q6GMV2), PRPS1 (P60891), SELENBP1 (Q13228_Q13228.4), and OXSR1 (O95747). One significantly associated protein group GOLPH3 (Q9H4A6) had a negative coefficient, implying that higher levels may be associated with delays in CDRg increase. Additionally, VGF (O15240), not previously identified as a significant biomarker of AD in plasma, was just outside significance (p-adj<0.1 after BH correction) and was also negatively associated with the time-to-CDRg increase.

To further assess our time-to-event approach, a CPH model was evaluated using the latest draw available (nearest to but before the event) with delayed entry and age as timescale (Model 2). The CPH models did not show any significantly associated proteins after BH correction, demonstrating that the CTV model provided greater statistical power than the CPH models. However, amongst those in the top 20 of lowest nominal p-values of Model 2 were six proteins (CLNS1A, CRISPLD2, GOLPH3, OXSR1, PRPS1, SELENBP1) that were also significantly associated in the CTV model. A different CPH model, one without delayed entry and age of the blood draw as a covariate (Model 3), was used to generate survival curves, with positive and negative association examples with time-to-CDRg increase shown in (FIG. 8D-E).

The proteins associated with dementia progression are of particular interest. They may provide development of a model to predict individuals that are at risk of rapid cognitive decline. Such a model could be used to aid treatment decisions in patients.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method of diagnosing Alzheimer's disease, the method comprising:
contacting a biofluid sample from a subject with one or more physicochemically distinct particles to form a plurality of biomolecule coronas;
obtaining a data set comprising protein or peptide information from the plurality of biomolecule coronas;
identifying, from the data set, an abundance of one or more biomarkers provided in Table 6 or Table 7; and
using a classifier to identify the biofluid sample as being indicative of a biological state comprising a healthy state or Alzheimer's disease (AD), based on the abundance of the one or more biomarkers in the data set.

2. The method of claim 1, wherein the one or more biomarkers comprise one or more of MBP, PLIN4, PKHG1, EIF1:EIF1B, CRBG3, ANTR1, PUR6, LUM, C1QT1, ENOB, DKK3, GPSM3, SYAC, HSP74, MICA1, APOD, LRC32, CSPG2, and OSTCN.

3. The method of claim 1, wherein the one or more biomarkers comprise MBP, PLIN4, PKHG1, EIF1:EIF1B, CRBG3, ANTR1, PUR6, LUM, C1QT1, ENOB, DKK3, GPSM3, SYAC, HSP74, MICA1, LRC32, CSPG2, and OSTCN.

4. The method of claim 1, wherein the one or more biomarkers comprise one or more of MBP and OSTCN.

5. The method of claim 1, wherein the one or more biomarkers comprise four or more biomarkers.

6. The method of claim 1, wherein the one or more biomarkers further comprise an additional biomarker comprising pTau (e.g., pTau-181 or pTau-217).

7. The method of claim 1, wherein the classifier comprises a machine learning algorithm.

8. The method of claim 7, wherein the machine learning algorithm comprises a logistic regression-based machine learning model.

9. A method for assessing a likelihood of dementia progression, the method comprising:
assaying a biofluid sample to obtain a data set comprising protein or peptide information;
identifying, from the data set, an abundance of one or more biomarkers; and
assessing a rate of dementia progression in the biofluid sample, based on the abundance of the one or more biomarkers.

10. The method of claim 9, wherein the one or more biomarkers comprise one or more of CRISPLD2, CLNS1A, BLVRB, SMYD5, PRPS1, SELENBP1, OXSR1, VGF, and GOLPH3.

11. The method of claim 10, wherein the CRISPLD2, CLNS1A, BLVRB, SMYD5, PRPS1, SELENBP1, OXSR1, or a combination thereof are associated with the biofluid sample having an increased rate of dementia progression.

12. The method of claim 10, wherein the CRISPLD2, CLNS1A, or a combination thereof are associated with the biofluid sample having an increased rate of dementia progression.

13. The method of claim 12, wherein the increased rate of dementia progression is associated with a shorter time to clinical dementia rating global (Cargo) increase.

14. The method of claim 10, wherein the GOLPH3, VGF, or a combination thereof are associated with the biofluid sample having a decreased rate of dementia progression.

15. The method of claim 14, wherein the decreased rate of dementia progression is associated with a delay in Cargo increase.

16. The method of claim 1, wherein the classifier comprises time-to-event analysis.

17. The method of claim 1, wherein the classifier comprises Cox proportion hazards (CPH) models, Cox time-varying (CTV) regression models, or a combination thereof.

18. The method of claim 1, wherein contacting comprises incubating the biofluid sample with the one or more physicochemically distinct particles.

19. The method of claim 1, wherein obtaining the data set comprises detecting the proteins of the biomolecule coronas by mass spectrometry.

20. The method of claim 1, wherein the biofluid sample comprises a blood sample, a serum sample, or a plasma sample.
